(19) United States
(12) Patent Application Publication
(10) Pub. No.: US 2025/0059591 A1
(43) Pub. Date: Feb. 20, 2025

(54) PCT/US22/79410

(71) Applicant: St. Jude Children's Research Hospital, Inc., Memphis, TN (US)

(72) Inventors: Paul G. THOMAS, Memphis, TN (US); Stephen GOTTSCHALK, Germantown, TN (US); Taylor WILSON, Memphis, TN (US); Jeremy Chase CRAWFORD, Memphis, TN (US); Hyunjin KIM, Memphis, TN (US)

(73) Assignee: St. Jude Children's Research Hospital, Inc., Memphis, TN (US)

(21) Appl. No.: 18/707,999

(22) PCT Filed: Nov. 7, 2022
(86) PCT No.: PCT/US2022/079410

§ 371 Date: May 7, 2024

Related U.S. Application Data
(60) Provisional application No. 63/353,145, filed on Jun. 17, 2022.
(60) Provisional application No. 63/276,999, filed on Nov. 8, 2021.

(51) Int. Cl. C12Q 1/6809; A61K 35/17; C12N 5/0783; C12Q 1/6869

(52) U.S. Cl. CPC C12Q 1/6809; A61K 35/17; C12N 5/0636; C12Q 1/6869; C12Q 2600/158

(57) ABSTRACT

The present disclosure relates generally to methods for generating a pre-effector gene signature for determining the C cytotoxic effector potential of a preparation of chimeric antigen receptor (CAR) T cells comprising measuring single-cell gene expression data and endogenous T cell receptor (TCR) sequencing data of the preparation of CAR T cells. The present invention relates also to methods for determining the cytotoxic effector potential of a preparation of CAR T cells or a subset of CAR T cells therefrom as well as methods for treating a cancer in a subject in need thereof by administering a preparation of CAR T cells determined to have a cytotoxic effector potential.

### CROSS-REFERENCE TO RELATED APPLICATIONS

This application claims priority to U.S. Provisional Application No. 63/276,999, filed Nov. 8, 2021, and U.S. Provisional Application No. 63/353,145, filed Jun. 17, 2022, the disclosures of which are herein incorporated by reference in their entirety.

### GOVERNMENT FUNDING

This invention was made with government support under AI136514 awarded by National Institutes of Health. The government has certain rights in the invention.

### FIELD OF THE INVENTION

The present disclosure relates generally to methods for generating a pre-effector gene signature for determining the cytotoxic effector potential of a preparation of chimeric antigen receptor (CAR) T cells comprising measuring single-cell gene expression data and endogenous T cell receptor (TCR) sequencing data of the preparation of CAR T cells. The present invention relates also to methods for determining the cytotoxic effector potential of a preparation of CAR T cells or a subset of CAR T cells therefrom as well as methods for treating a cancer in a subject in need thereof by administering a preparation of CAR T cells determined to have cytotoxic effector potential.

### BACKGROUND

Chimeric antigen receptor-modified (CAR) T cells have improved treatment outcomes for many cancer types, primarily hematological malignancies. In B cell acute lymphoblastic leukemia (B-ALL), initial use of an anti-CD19 CAR T cell product followed by allogeneic hematopoietic stem cell transplantation has been able to fully cure some patients with relapsed or refractory disease. While response rates at the time of treatment are quite high, a subset of CAR T cell-treated patients do not mount or sustain a robust therapeutic response. There are many factors that can contribute to treatment failure, including lack of cellular expansion post-infusion, diminished effector differentiation, and an absence of long-term persistence to adequately control lymphoblast proliferation. These factors are likely, at least in part, due to intrinsic features of the CAR T cells themselves.

Chimeric antigen receptor modified (CAR) T cells have been successful in the treatment of B-cell acute lymphoblastic leukemia (B-ALL). However, challenges, related to lack of CAR T cell effector response, expansion, or persistence, remain. CAR T cell therapy products have, thus far, been mostly evaluated in bulk, without assessment of the possible heterogeneity in effector potential between cells. Conceivably, only a subset of the pre-infusion product differentiates into optimal effectors. There is a need for detailed evaluation of distinct transcriptional subsets within the CAR T cell pre-infusion product that will identify intrinsic cellular features leading to CAR T cell expansion, effector development, and therapeutic success.

### SUMMARY OF THE INVENTION

In one aspect, the invention relates to a method for generating a pre-effector gene signature for determining the cytotoxic effector potential of a preparation of chimeric antigen receptor (CAR) T cells, the method comprising: a) measuring single-cell gene expression data and endogenous T cell receptor (TCR) sequencing data of the preparation of CAR T cells; b) administering the preparation of CAR T cells to a subject; c) isolating post-infusion CAR T cells at one or more time points after the administration step b); d) acquiring single-cell gene expression data and endogenous TCR sequencing data of the cells isolated in step c); e) using the endogenous TCR sequencing data obtained in steps a) and d) to determine which CAR T cells from the preparation of CAR T cells are cytotoxic effector precursors by determining which of the CAR T cells of the preparation of CAR T cells share a clonally related TCR with post-infusion CAR T cells that differentiated into cytotoxic effector CAR T cells; and f) generating a pre-effector gene signature by determining which set of genes are differentially expressed in the cytotoxic effector precursors as compared to other or all other CAR T cells from the preparation.

In certain embodiments, the post-infusion CAR T cells are isolated from peripheral blood mononuclear cells (PBMCs), bone marrow, or both PBMCs and bone marrow.

In certain embodiments, the post-infusion CAR T cells are isolated about 1 week, about 2 weeks, about 3 weeks, about 4 weeks, about 8 weeks, about 3 months, and/or about 6 months after administering the preparation of CAR T cells to the subject. In certain embodiments, the post-infusion CAR T cells are isolated at least once at about 2-3 weeks after administering the preparation of CAR T cells to the subject. In certain embodiments, the post-infusion CAR T cells are isolated 1, 2, 3, 4, 5, 6, 7, 8, 9, 10, 12, 15, or 20 times after administering the preparation of CAR T cells to the subject.

In one aspect, the invention relates to a method of determining the cytotoxic effector potential of a preparation of CAR T cells, the method comprising: a) acquiring single-cell gene expression data of the preparation of CAR T cells for a set of differentially expressed markers which are expressed at different levels in CAR T cells which differentiate into cytotoxic effector cells upon administration to a subject, and b) calculating the cytotoxic effector potential indicating the likelihood of the CAR T cell preparation, when administered, to give rise to post-infusion CAR T cells and lineages thereof with cytotoxic effector function, wherein the cytotoxic effector potential of individual cells is assessed by training a machine learning algorithm to determine which cells in the preparation are effector precursors.

In certain embodiments, the cytotoxic effector potential of individual cells is determined by training a machine learning classifier with a training set of differentially expressed genes to generate a value reflecting the probability the analyzed cell is a cytotoxic effector precursor.

In certain embodiments, the method of determining the cytotoxic effector potential of a preparation of CAR T cells further comprising calculating the proportion of the CAR T cells in the preparation with cytotoxic effector potential as determined in step b) of the method of determining the cytotoxic effector potential of a preparation of CAR T cells.

In certain embodiments, the training set of differently expressed genes comprises the genes of Table 1, Table 2, Table 3, Table 4, FIG. 1B, genes of clusters 1, 3, 5, 7, 8, 12 and/or 14 of FIG. 1B and/or 3, 8, and/or 14 of FIG. 1B.

In certain embodiments, the machine learning algorithm is trained using a training set of differently expressed genes comprises the top 1, 2, 3, 4, 5, 10, 15, 20, 25, 30, 35, 40, 45, 50, 55, 60, 65, 70, 75, 80, 85, 90, 95, or 100 genes (or integers in between) of the pre-effector gene signature generated by the method of generating a pre-effector gene signature for determining the cytotoxic effector potential of a preparation of chimeric antigen receptor (CAR) T cells.

In certain embodiments, the training set of differently expressed genes comprises the top 3 to 1,100 genes of the pre-effector gene signature generated by the method of generating a pre-effector gene signature for determining the cytotoxic effector potential of a preparation of chimeric antigen receptor (CAR) T cells.

In one aspect, the invention relates to a method of determining the cytotoxic effector potential of a preparation of CAR T cells or a subset of CAR T cells therefrom, the method comprising: a) acquiring single-cell gene expression data of the preparation of CAR T cells for a set of differentially expressed markers which are expressed at different levels in CAR T cells which differentiate into cytotoxic effector cells upon administration to a subject, and b) determining the cytotoxic effector potential of a preparation of CAR T cells or a subset of CAR T cells therefrom by using flow cytometry to identify the CAR T cells of the preparation of CAR T cells that express the gene product of the set of differentially expressed markers.

In one aspect, the invention relates to a method of determining the cytotoxic effector potential of a preparation of CAR T cells or a subset of CAR T cells therefrom, the method comprising: a) acquiring single-cell gene expression data of the preparation of CAR T cells for a set of differentially expressed markers which are expressed at different levels in CAR T cells which differentiate into cytotoxic effector cells upon administration to a subject, and b) determining the cytotoxic effector potential of a preparation of CAR T cells or a subset of CAR T cells therefrom by measuring protein expression of the gene product of the set of differentially expressed markers.

In certain embodiments, the protein expression is measured by western blot and/or by ELISA.

In one aspect, the invention relates to a method of determining the cytotoxic effector potential of a preparation of CAR T cells or a subset of CAR T cells therefrom, the method comprising: a) acquiring single-cell gene expression data of the preparation of CAR T cells for a set of differentially expressed markers which are expressed at different levels in CAR T cells which differentiate into cytotoxic effector cells upon administration to a subject, and b) determining the cytotoxic effector potential of a preparation of CAR T cells or a subset of CAR T cells therefrom by measuring gene expression of the set of differentially expressed markers.

In one aspect, the invention relates to a method of determining the cytotoxic effector potential of a preparation of CAR T cells or a subset of CAR T cells therefrom, the method comprising: a) acquiring single-cell gene expression data of the preparation of CAR T cells or for a set of differentially expressed markers which are expressed at different levels in CAR T cells which differentiate into cytotoxic effector cells upon administration to a subject; b) comparing the single-cell gene expression data of the preparation of CAR T cells or a subset of CAR T cells therefrom with the pre-effector gene signature generated by the method of generating a pre-effector gene signature for determining the cytotoxic effector potential of a preparation of chimeric antigen receptor (CAR) T cells; and c) determining the preparation of CAR T cells or a subset of CAR T cells therefrom has cytotoxic effector potential if the preparation of CAR T cells or a subset of CAR T cells therefrom shares at least one or more marker genes from the pre-effector gene signature.

In certain embodiments, the one or more marker genes comprises one or more genes of Table 1, Table 2, Table 3, Table 4, FIG. 1B, genes of clusters 1, 3, 5, 7, 8, 12 and/or 14 of FIG. 1B and/or 3, 8, and/or 14 of FIG. 1B.

In certain embodiments, the one or more marker genes comprises the top 1, 2, 3, 4, 5, 10, 15, 20, 25, 30, 35, 40, 45, 50, 55, 60, 65, 70, 75, 80, 85, 90, 95, or 100 genes (or integers in between) of the pre-effector gene signature generated by the method of generating a pre-effector gene signature for determining the cytotoxic effector potential of a preparation of chimeric antigen receptor (CAR) T cells.

In certain embodiments, the one or more marker genes comprises the top 3 to 1,0341,100 genes of the pre-effector gene signature generated by the method of generating a pre-effector gene signature for determining the cytotoxic effector potential of a preparation of chimeric antigen receptor (CAR) T cells.

In one aspect, the invention relates to a method for treating a disease or condition in a subject in need thereof, the method comprising administering a preparation of chimeric antigen receptor (CAR) T cells comprising a set of differentially expressed markers.

In certain embodiments, the disease or condition is cancer.

In one aspect, the invention relates to a method of selecting a preparation of chimeric antigen receptor (CAR) T cells comprising selecting a preparation of CAR T cells comprising CAR T cells comprising a set of differentially expressed markers.

In certain embodiments, the pre-effector gene signature and/or set of differentially expressed markers comprises one or more marker genes selected from the genes of Table 1, 2, 3, and/or 4 and/or FIG. 1B, and wherein said one or more marker genes are expressed at a different level in CAR T cells which differentiate into cytotoxic effector cells upon administration to a subject.

In certain embodiments, the pre-effector gene signature and/or set of differentially expressed markers comprises one or more marker genes selected from the genes of clusters 1, 3, 5, 7, 8, 12 and/or 14 of FIG. 1B or 3, 8, and/or 14 of FIG. 1B.

In certain embodiments, the pre-effector gene signature and/or set of differentially expressed markers comprises one or more marker genes selected from the genes of Table 3.

In certain embodiments, the pre-effector gene signature and/or set of differentially expressed markers comprises the marker genes of Table 3.

In certain embodiments, the set of differentially expressed markers is a set of one or more genes of the pre-effector gene signature generated by the method of generating a pre-effector gene signature for determining the cytotoxic effector potential of a preparation of chimeric antigen receptor (CAR) T cells.

In certain embodiments, the set of differentially expressed markers comprises one or more marker genes selected from LTB, EOMES, GNLY, GZMA, GZMH, GZMK, KLRD1, IFNG, CD52, CD74, CD86, LAG3, CASP8, TOX, TIGIT, CD27, SELL, CD44, CD70, HLA-DQA1, HLA-DR, HLA-DQ, PRF1, CDC20, PLK1, MKI67, and IL-7R, and wherein said one or more marker genes are expressed at a different level in CAR T cells which differentiate into cytotoxic effector cells upon administration to a subject.

In certain embodiments, the set of differentially expressed markers comprises one or more marker genes selected from EOMES, GNLY, GZMH, GZMK, KLRD1, and IFNG genes. In certain embodiments, the set of differentially expressed markers comprises one or more marker genes selected from EOMES, GNLY, GZMH, GZMK, and IFNG genes. In certain embodiments, the set of differentially expressed markers comprises one or more marker genes selected from LTB, GZMA, GZMK, PRF1, CDC20, PLK1, and MKI67 genes. In certain embodiments, the set of differentially expressed markers comprises one or more marker genes selected from CD52, CD74, CD86, and LAG3 genes. In certain embodiments, the set of differentially expressed markers comprises one or more marker genes selected from LEF1, IL7R, SELL, and CD27 genes. In certain embodiments, the set of differentially expressed markers comprises one or more marker genes selected from GZMK, KLRD1, IFNG, GZMH, GNLY, and EOMES genes. In certain embodiments, the set of differentially expressed markers comprises one or more marker genes selected from GZMK, KLRD1, IFNG, GZMH, GNLY, and EOMES genes. In certain embodiments, the set of differentially expressed markers comprises decreased expression of one or more marker genes selected from CD7, CD70, and CD86. In certain embodiments, the set of differentially expressed markers comprises decreased expression of one or more marker genes selected from IFITM1, IFITM2, and IFITM3 genes or IFITM1 and IFITM2 genes. In certain embodiments, the set of differentially expressed markers comprises increased expression of TIGIT and/or LAG3 genes. In certain embodiments, the set of differentially expressed markers comprises decreased expression of CD27 gene. In certain embodiments, the set of differentially expressed markers comprises TIGIT, SELL, and CD27 genes. In certain embodiments, the set of differentially expressed markers comprises expression or increased expression of TIGIT gene, reduced expression of SELL gene and reduced or no expression of DC27 gene.

In certain embodiments, the methods of the present disclosure further comprising enriching the preparation of CAR T cells for cytotoxic effector precursors.

In certain embodiments, the one or more surface proteins are selected from CD7, KLRD1, CD70, CD44, CD25, CD127, CD55, CD82, CD52, CD74, CD86, LAG3, TIGIT, CD62L, and CD27. In certain embodiments, the one or more surface proteins are selected from TIGIT and/or LAG3. In certain embodiments, the one or more surface proteins are selected from CD62L, CD127, and CD27. In certain embodiments, the one or more surface proteins are selected from CD62L TIGIT, and CD27.

In certain embodiments, wherein the enrichment is conducted using a method selected from fluorescence-activated cell sorting (FACS), magnetic-activated cell sorting (MACS), microfluidics and buoyancy activated cell sorting (BACS).

In one aspect, the invention relates to a method for enriching a preparation of chimeric antigen receptor (CAR) T cells for CAR T cells that differentiate into cytotoxic effector cells upon administration to a subject using one or more surface proteins encoded by marker genes of a pre-effector gene signature comprising genes which are expressed higher on cytotoxic effector precursors and lower or not at all on other CAR T cells from the preparation.

In certain embodiments, the marker genes comprise at least 1, 2, 3, 4, 5, 6, 7, 8, 9, 10, 11, 12, 13, 14, or 15 genes that encode surface proteins of the pre-effector gene signature generated by the method of generating a pre-effector gene signature for determining the cytotoxic effector potential of a preparation of chimeric antigen receptor (CAR) T cells.

In certain embodiments, the one or more surface proteins are selected from CD7, KLRD1, CD70, CD44, CD25, CD127, CD55, CD82, CD52, CD74, CD86, LAG3, TIGIT, CD62L, and CD27. In certain embodiments, the one or more surface proteins are selected from TIGIT and/or LAG3. In certain embodiments, the one or more surface proteins are selected from CD62L, CD127, and CD27. In certain embodiments, the one or more surface proteins are selected from CD62L TIGIT, and CD27.

In certain embodiments, the enrichment is conducted using a method selected from fluorescence-activated cell sorting (FACS), magnetic-activated cell sorting (MACS), microfluidics and buoyancy activated cell sorting (BACS).

In certain embodiments, the pre-effector gene signature comprises expression levels of genes that correspond to cell-surface expressed proteins.

In certain embodiments, the method further comprising using a machine learning algorithm. In certain embodiments, the machine learning algorithm is Support Vector Machines. In certain embodiments, the machine learning algorithm is k-nearest neighbors (KNN), gradient boosting algorithm (GBM), or SVM Linear/Radial algorithm.

In certain embodiments, the endogenous TCR sequencing data are V(D)J sequencing data.

In certain embodiments, the methods of the present disclosure further comprising administering the preparation of CAR T cells or the enriched preparation of CAR T cells that differentiate into cytotoxic effector cells to the subject.

In certain embodiments, the methods of the present disclosure further comprising acquiring a preparation of CAR T cells.

In certain embodiments, the subject has cancer. In certain embodiments, cancer is B cell acute lymphoblastic leukemia (B-ALL).

In certain embodiments, the CAR T cell is an CD19− or an-CD123-CAR T cell. In certain embodiments, the CAR T cell comprises a CAR comprising a costimulatory domain.

In certain embodiments, the costimulatory domain is a 4-1BB costimulatory domain.

In certain embodiments, the CAR T cells are autologous. In certain embodiments, the CAR T cells are allogenic.

In one aspect, the invention relates to a method for treating a cancer in a subject in need thereof, the method comprising administering a preparation of chimeric antigen receptor (CAR) T cells determined to have cytotoxic effector potential determined by the methods of the present disclosure.

In one aspect, the invention relates to a method for treating a cancer in a subject in need thereof, the method comprising administering an enriched preparation of chimeric antigen receptor (CAR) T cells generated by the methods of the present disclosure.

In certain embodiments, the subject is human.

In certain embodiments, the set of differentially expressed markers are expressed at different levels in CAR T cells which differentiate into cytotoxic effector cells upon administration to a subject as compared to all other CAR T cells in a CAR T cell preparation.

In certain embodiments, the set of differentially expressed markers are expressed at different levels in CAR T cells which differentiate into cytotoxic effector cells upon administration to a subject as compared to CAR T cells which do not differentiate or less likely to differentiate into cytotoxic effector cells upon administration to a subject.

In certain embodiments, the set of differentially expressed markers are expressed at different levels in CAR T cells which differentiate into cytotoxic effector cells upon administration to a subject as compared to CAR T cells which exhibit no evidence of lineage-specific differentiation into cytotoxic effector cells upon administration to a subject.

In one aspect, the invention relates to a preparation of CAR T cells with cytotoxic effector potential determined by the methods of the present disclosure or enriched by the methods of the present disclosure.

In one aspect, the invention relates to a kit for determining the cytotoxic effector potential of a preparation of CAR T cells comprising a set or reagents that detects the level of one or more genes listed in Table 1, 2, 3, and/or 4 and/or FIG. 1B or the genes identified in any one of the embodiments of the present disclosure and optionally instructions for using said kit; wherein said instructions for use provide that if one or more of the detected expression levels is different from, e.g., greater than a reference level, the subject is more likely to be a cytotoxic effector precursor.

In certain embodiments, the set of reagent detects the expression of mRNA expressed from said set of genes. In certain embodiments, the set of reagents detects the expression of polypeptides encoded by said set of genes.

In one aspect, the invention relates to non-transitory computer-readable medium configured to communicate with one or more processor(s) of a computational system, the non-transitory computer-readable medium including instructions thereon, that when executed by the processor(s), cause the computational system to perform the following steps. Steps can be performed in a variety of sequences and can include interleaving steps not listed.

In certain embodiments, the instructions can cause the computational system to receive, as inputs, a preparation of CAR T cell gene expression dataset comprising single cell gene expression data of CAR T cells, endogenous TCR sequencing data each associated with respective single cell gene expression data of the preparation of CAR T cell single cell gene expression dataset, a post-infusion CAR T cell gene expression dataset comprising single cell gene expression data of CAR T cells originating from subjects post-infusion, and endogenous TCR sequencing data each associated with respective single cell gene expression data of the post-infusion CAR T cell gene expression dataset.

In certain embodiments, the preparation of CAR T cell gene expression dataset can include single cell gene expression data of CAR T cells that originate from the subject's pre-infusion and transduced with a CAR.

In certain embodiments, the post-infusion CAR T cell gene expression dataset can include single cell gene expression of CAR T cells originating from the subjects at multiple times post-infusion.

In certain embodiments, the multiple times post-infusion can include approximately 2 weeks post-infusion and approximately 3 weeks post-infusion.

In certain embodiments, the multiple times post-infusion can include approximately 1 week, approximately 3 weeks, approximately 4 weeks, approximately 8 weeks, approximately 3 months, and/or approximately 6 months post-infusion.

In certain embodiments, the instructions can cause the computational system to identify, based on the post-infusion CAR T cell gene expression dataset, single cell gene expression data of cytotoxic effector CAR T cells. This step can be executed according to sub-steps. The sub-steps can be performed in various sequences, and the instructions can include interleaving steps not listed. In certain embodiments, the instructions can cause the computational system to cluster the single cell gene expression data of the post-infusion CAR T cell gene expression dataset using a clustering algorithm, resulting in a plurality of clusters such that each cluster in the plurality of clusters comprises similar single cell gene expression data within the cluster and such that single cell gene expression data differs across differing clusters. In certain embodiments, the instructions can cause the computational system to determine one or more functional effector cluster(s) of the plurality of clusters such that the one or more functional effector cluster(s) are associated with a functional effector T cell functional state. In certain embodiments, the instructions can cause the computational system to identify the single cell gene expression data of cytotoxic effector CAR T cells such that the single cell gene expression data of cytotoxic effector CAR T cells are within the one or more cluster(s) associated with the functional effector T cell functional state.

In certain embodiments, the plurality of clusters can further include single cell gene expression data of the preparation of CAR T cell gene expression dataset.

In certain embodiments, the clustering algorithm can include a dimensionality reduction algorithm. The dimensionality reduction algorithm can include uniform manifold approximation and projection (UMAP), principal component analysis (PCA), t-distributed stochastic neighbor embedding (tSNE), and/or Pseudotime analysis. The dimensionality reduction algorithm preferably includes UMAP.

In certain embodiments, the one or more functional effector cluster(s) can be determined according to the following sub-steps. The sub-steps can be performed in various sequences, and the instructions can include interleaving steps not listed. In certain embodiments, the instructions can cause the computational system to compare, for each cluster of the plurality of clusters, single cell gene expression data of the cluster to every other cluster to thereby identify differentially expressed markers associated with the cluster. In certain embodiments, the instructions can cause the computational system to determine the one or more functional effector cluster(s) such that differentially expressed markers of each of the one or more functional effector clusters(s) comprise known genes associated with the functional effector T cell functional state.

In certain embodiments, the one or more functional effector cluster(s) can further be determined according to the following sub-steps. The sub-steps can be performed in various sequences, and the instructions can include interleaving steps not listed. In certain embodiments, the instructions can cause the computational system to determine, for each of the clusters of the plurality of clusters, a ratio equal to CAR T cells represented only in the cluster over CAR T cells represented in the entire plurality of clusters. In certain embodiments, the instructions can cause the computational system to determine the one or more functional effector cluster(s) such that the ratio of each of the one or more functional effector clusters(s) is greater than the ratio of clusters not identified as a functional effector cluster.

In certain embodiments, the instructions can cause the computational system to identify clonal TCR sequencing data such that the clonal TCR sequencing data is (i) associated with single cell gene expression data of the preparation of CAR T cell gene expression dataset, and is (ii) clonally related to endogenous TCR sequencing data associated with each of the single cell gene expression data of cytotoxic effector CAR T cells.

In certain embodiments, the instructions can cause the computational system to identify cytotoxically effective precursor gene expression data such that the cytotoxically effective precursor gene expression data comprises single cell gene expression data of the preparation of CAR T cell gene expression dataset associated with the clonal TCR sequencing data.

In certain embodiments, the instructions can cause the computational system to identify, based at least in part on the cytotoxically effective precursor gene expression data, a pre-effector gene signature comprising markers for determining a cytotoxic effector potential of CAR T cells. In certain embodiments, this step can be executed according to sub-steps. The sub-steps can be performed in various sequences, and the instructions can include interleaving steps not listed. In certain embodiments, the instructions can cause the computational system to identify comparator single cell gene expression data of the preparation of CAR T cell gene expression dataset such that endogenous TCR sequencing data associated with the comparator single cell gene expression data lacks any clonal relationship to endogenous TCR sequencing data associated with single cell gene expression data of the cytotoxically effective CAR T cells of the post-infusion CAR T cell gene expression dataset. In certain embodiments, the instructions can cause the computational system to identify, based on a comparison of the cytotoxically effective precursor gene expression data to the comparator single cell gene expression data, upregulated genes and/or downregulated genes in relation to the cytotoxically effective precursor gene expression data. In certain embodiments, the instructions can cause the computational system to identify the pre-effector gene signature such that the pre-effector gene signature comprises a subset of the upregulated genes and/or downregulated genes that are differentially expressed.

In certain embodiments, the subset of the upregulated genes and/or downregulated genes can include about 20 to about 100 genes.

In certain embodiments, the subset of the upregulated genes and/or downregulated genes can include the top 1, 2, 3, 4, 5, 10, 15, 20, 25, 30, 35, 40, 45, 50, 55, 60, 65, 70, 75, 80, 85, 90, 95, or 100 genes (or integers in between) of upregulated genes and/or downregulated genes.

In certain embodiments, the subset of the upregulated genes and/or downregulated genes can include the top 3 to 1,100 genes of upregulated genes and/or downregulated genes.

In certain embodiments, the subset of the upregulated genes and/or downregulated genes can include the genes of Table 1, Table 2, Table 3, Table 4, FIG. 1B, genes of clusters 1, 3, 5, 7, 8, 12 and/or 14 of FIG. 1B and/or 3, 8, and/or 14 of FIG. 1B.

In certain embodiments, the cytotoxic effector potential can include a numerical value indicating likelihood of a CAR T cell, when administered, to give rise to a lineage that results in functional effector post-infusion T cells.

In certain embodiments, the instructions can cause the computational system to provide, as an output, the pre-effector gene signature.

In certain embodiments, the instructions can cause the computational system to provide the markers of the pre-effector gene signature as a training input to a machine learning algorithm, thereby resulting in a classifier. The classifier can be configured to receive, as an input, single cell gene expression data of a CAR T cell, and provide, as an output, the cytotoxic effector potential of the CAR T cell represented by the single cell gene expression data that is received as input by the classifier.

In one aspect, the invention relates to non-transitory computer-readable medium configured to communicate with one or more processor(s) of a computational system, the non-transitory computer-readable medium including instructions thereon, that when executed by the processor(s), cause the computational system to perform the following steps. Steps can be performed in a variety of sequences and can include interleaving steps not listed.

In certain embodiments, the instructions can cause the computational system to receive, as an input, single-cell gene expression data of a CAR T cell. In certain embodiments, the instructions can cause the computational system to provide, as an output, a cytotoxic effector potential indicating likelihood of the CAR T cell, when administered, to give rise to CAR T cells and lineages thereof that results in functional effector post-infusion T cells.

In certain embodiments, the instructions may not require endogenous T cell receptor (TCR) sequencing data associated with the single-cell gene expression data in order to provide the cytotoxic effector potential.

In certain embodiments, the instructions can cause the computational system to identify the CAR T cell as a cytotoxically effective precursor.

In certain embodiments, the instructions can be based on a machine learning algorithm provided training set of markers of the pre-effector gene signature as a training input.

In certain embodiments, the training set of markers can include the genes of Table 1, Table 2, Table 3, Table 4, FIG. 1B, genes of clusters 1, 3, 5, 7, 8, 12 and/or 14 of FIG. 1B and/or 3, 8, and/or 14 of FIG. 1B.

In certain embodiments, the training set of markers can include the top 1, 2, 3, 4, 5, 10, 15, 20, 25, 30, 35, 40, 45, 50, 55, 60, 65, 70, 75, 80, 85, 90, 95, or 100 genes (or integers in between) of the pre-effector gene signature.

In certain embodiments, the training set of markers can include the top 3 to 1,100 genes of the pre-effector gene signature.

In one aspect, the invention relates to non-transitory computer-readable medium configured to communicate with one or more processor(s) of a computational system, the non-transitory computer-readable medium including instructions thereon, that when executed by the processor(s), cause the computational system to perform the following steps. Steps can be performed in a variety of sequences and can include interleaving steps not listed. In certain embodiments, the instructions can cause the computational system to receive, as an input, a preparation of chimeric antigen receptor (CAR) T cell gene expression dataset comprising single cell gene expression data of CAR T cells of a preparation of CAR T cells. In certain embodiments, the instructions can cause the computational system to determine a cytotoxic effector potential for each single cell gene expression data of the preparation of CAR T cell gene expression dataset, wherein the cytotoxic effector potential indicates likelihood of a CAR T cell, when administered, to give rise to a lineage that results in functional effector post-infusion CAR T cells. In certain embodiments, the instructions can cause the computational system to calculate, based on the cytotoxic effector potentials, a gene set module score for the preparation of CAR T cells, wherein the gene set module score indicates likelihood of the preparation of CAR T cells, when administered, to give rise to a clinically effective quantity of functional effector post-infusion T cells.

In certain embodiments, the gene set module score can be calculated according to the following sub-steps. The sub-steps can be performed in various sequences, and the instructions can include interleaving steps not listed. In certain embodiments, the instructions can cause the computational system to determine a threshold value for the cytotoxic effector potential such that single cell gene expression data determined to have a cytotoxic effector value above the threshold represent CAR T cells having a high likelihood to give rise to a lineage that results in functional effector post-infusion T cells. In certain embodiments, the instructions can cause the computational system to calculate a numerator value that is equal to number of CAR T cells of the preparation of CAR T cells represented by the single cell gene expression data having a cytotoxic effector potential above the threshold value. In certain embodiments, the instructions can cause the computational system to calculate a denominator value that is equal to a total number of CAR T cells of the preparation represented by the single cell gene expression data of the preparation of CAR T cell gene expression dataset. In certain embodiments, the instructions can cause the computational system to calculate the gene set module score based on a ratio of the numerator value to the denominator value.

### BRIEF DESCRIPTION OF THE DRAWINGS

This application claims priority to U.S. Provisional Application No. 63/276,999 and U.S. Provisional Application No. 63/353,145; color version of FIGS. 1-18 are available in the provisional application files with the United States Patent and Trademark Office.

FIGS. 1A-1C depict identification of subsets within pre- and post-CAR T cells. FIG. 1A: Uniform manifold approximation and projection (UMAP) plot with shared nearest neighbor clustering of 184,791 pre- and post-infusion CAR T cells across all patients, indicated by 21 transcriptional clusters. FIG. 1B: Chart depicting cluster numbers as indicated in FIG. 1A, with key genes used to characterize the transcriptional profile of each cluster, the functional groups each cluster was assigned based on the specific transcriptional profile, the number of cells in each cluster, the percent GMP composition of each cluster, and the percent of cells inferred to be CD8+ of each cluster. Percent of cells in a cluster that were CD8+ was inferred using SignacX. FIG. 1C: Dot plots showing the relative expression of genes characteristic to relevant cellular processes as listed. Dot size correlates with the percent of cells expressing each gene.

FIGS. 2A-2F show single-cell transcriptomics of pre- and post-infusion CAR T cells. FIGS. 2A-2B: UMAP of pre-(labeled GMP) and post-infusion (labeled PI) CAR T cells wherein CAR product/post-infusion status (FIG. 2A) or CD4/CD8 status (FIG. 2B) are indicated by different shades of grey. FIG. 2C: Violin plot comparing expression of GZMK in clusters 3 and 8. FIG. 2D: UMAP wherein cellular density is indicated by difference shades of grey. FIG. 2E: Dot plot visualizing the relative expression of a subset of genes that were differentially expressed across all clusters. Dot size corresponds to percent of cells expressing a gene. FIG. 2F: Heatmap of the top 5 differentially expressed genes from each transcriptional cluster.

FIGS. 3A-3E show expression of effector and dysfunctional T cell genes over-time correlates with kinetics of CAR T cell subsets. FIG. 3A: Relative proportion of functional groups as outlined in FIG. 1 across each pre-infusion samples and post-infusion timepoints, where functional groups are indicated by different shades of grey. FIG. 3B: UMAP of pre- and post-infusion CAR cells, wherein different shades of grey indicating CAR product status or post-infusion timepoint. FIG. 3C: Heatmap of average gene expression across CAR cell timepoints. Included are genes associated with cytotoxic effector function and exhaustion or T cell dysfunction as indicated. FIG. 3D: Stacked bar plots of each cluster's contribution to the indicated timepoints, wherein clusters are indicated by different shades of grey. FIG. 3E: Violin plots depicting activity level of select regulons that were significantly different between transcriptional clusters 3 and 8. Asterisks indicate the degree of significance (****=padj<1E-15; ***=padj<1E-10 (>1E-15); **=padj<1E-5 (>1E-10); *=padj<0.05 (>1E-5)).

FIGS. 4A-4D show that pseudotime trajectory analysis identifies a subset of dysfunctional post-infusion CAR cells that arise directly from the CAR product as opposed to prolonged antigen exposure. FIGS. 4A-4B: Principal component analysis (PCA) plot depicting pseudotime trajectory analysis of 3,416 CAR cells. Different shades of grey indicate pseudotime state of the cells (FIG. 4A), different shades of grey indicate either CAR product or post-infusion status of the cells (FIG. 4B). FIG. 4C: PCA of pseudotime trajectory wherein different shades of grey indicate the relative expression of CASP8, LAG3, and TOX (top). Also included is a dot plot of comparing relative expression of CASP, LAG3, and TOX across pseudotime states (bottom). FIG. 4D: Dot plot comparing relative expression of effector genes (NKG7, GNLY, GZMB, and GZMK) across pseudotime time states.

FIGS. 5A-5B show characterization of CAR cells in pseudotime. FIG. 5A: PCA plot of CAR T cell transcriptional trajectory, wherein the pseudotime gradient is indicated by different shades of grey. FIG. 5B: PCA plots depicting each cluster in the pseudotime space.

FIGS. 6A-6E show tracking of endogenous TCR over time identifies CAR T cell lineages and their subsequent fates. FIG. 6A: Alluvial plot of CAR T cell lineages across the GMP and post-infusion timepoints. To define a lineage, the level of stringency necessary to accurately define a lineage (detailed further in FIG. 9) was determined. Each line corresponds to an individual CAR T cell lineage, illustrating the appearance of lineages across multiple timepoints. Due to space constraints, only immediately consecutive connections are visualized (e.g., excluding direct connections between GMP and Wk3 for instance). FIG. 6B (left): UMAP emphasizing post-infusion CAR T cell lineages. Arrows indicate the CD8+ CAR T cells of the same lineage, starting at the earliest post-infusion appearance of a lineage and ending at the final appearance of the lineage. Different shades of grey indicate cells without lineages across post-infusion time points and cells with different post-infusion time points. FIG. 6B (right): Alluvial plot depicting the cluster assigned to the earliest post-infusion appearance of a lineage and the cluster assigned to the final appearance of a lineage. FIG. 6C (left): UMAP emphasizing GMP CAR T cell lineages. Arrows indicate the CD8+ CAR T cells of the same lineage, starting at the appearance of the lineage in the GMP and ending at the final appearance of the lineage. Different shades of grey indicate cells without lineages track to the GMP product and cells according to their GMP status or post-infusion time point. FIG. 6C (right): Alluvial plot depicting the cluster assigned to the appearance of the lineage in the GMP and the cluster assigned to the final appearance of the lineage. FIG. 6D: Network plot of CAR T cell lineages. Dot size indicates clone sizes. Arrows link cells that share TCRs; showing either connections between GMP and post-infusion cells, or lineages between post-infusion cells. FIG. 6E: Circos plot showing the GMP clusters with lineages to post-infusion CD8+ functional effector clusters (3 and 8). Different shades of grey of the outermost ring indicates post infusion (PI) status or GMP status.

FIGS. 7A-7B show TCR lineage tracking. FIG. 7A: Cumulative clone sizes of GMP and post-infusion CAR T cell lineages and GMP and post infusion CAR T cells without observed lineages (controls) and their corresponding proportion of the overall number of unique lineages. FIG. 7B: UMAP of pre- and post-infusion CAR T cells as described in FIG. 8. Only CD4+ CAR T cells lineages are shown.

FIGS. 8A-8H show that effector GMP precursors have an identifiable signature pre-infusion. FIG. 8A: Dot plot comparing average expression of 14 genes differentially expressed between GMP effector precursors (as defined by αβTCR lineage tracing) and all other CD8 GMP CAR cells. FIG. 8B: AUC of the best performing iteration of a classifier trained on the top 100 differentially expressed genes between these GMP effector precursors and all other CD8 GMP cells. The single-cell dataset was randomly downsampled 1,000 times for this analysis. FIG. 8C: Flow cytometry data from an aliquot of patient 11's GMP infusion product, visualizing CD8+ CAR cells with the predicted effector precursor profile (TIGIT+, CD62Llo, and CD27lo) and the opposite non-effector associated surface profile (TIGIT−, CD62hi, and CD27hi). FIG. 8D: Bar plot comparing the proportion of bulk β TCRs sequenced that matched those from post-infusion CD8 transcriptional clusters. Differences are represented as log 2 fold change for the proportions of TCRs matching each cluster. FIG. 8E: Cumulative $ clone sizes of post-infusion cells that share β TCRs with GMP product cells sorted by the precursor effector signature or the opposing, non-effector signature. Graph represents the proportion of cells (y-axis) cumulatively encompassed by increasing clone sizes. FIG. 8F: Box plot comparing the proportion of GMP to post-infusion lineages that ended up in effector clusters 3 and 8 between responders and non-responders. FIG. 8G: UMAP based on SCENIC transcriptional factor regulatory network analysis conducted on effector precursors (labeled as “precursors”) and a random subset of other CD8+ GMP CAR T cells (labeled as “non-precursors”). FIG. 8H: Violin plots comparing activity levels of regulons based on SCENIC analysis. Asterisks indicate the degree of significance (****=padj<1E-15; ***=padj<1E-10 (>1E-15); **=padj<1E-5 (>1E-10); *=padj<0.05 (>1E-5)).

FIGS. 9A-9B show selection of surface targets for pre-effector surface phenotype validation. FIG. 9A: Histograms comparing expression of a subset of genes encoding surface proteins in effector precursors and non-effector precursors. FIG. 9B: Bar plots representing the inferred proportions of post-infusion CD8 transcriptional clusters linked to Patient 11 CAR product cells sorted to approximate either the pre-effector signature (PE) or the opposing, non-effector signature (NE). TCRβ chains were sequenced in bulk from each sort, and cluster-annotated post-infusion cells from CD8 clusters that shared those β sequences were used to infer the post-infusion transcriptional cluster those lineages would track to.

FIG. 10 shows kinetics of CAR T cell clusters. Stacked bar plot depicting the number of cells in each cluster and the relative contribution of each timepoint to the individual clusters.

FIGS. 11A-11B show an exemplary density plot and its associate histogram. FIG. 11A shows a density plot, which used an absolute module score threshold of 0.4 (specific to this particular module score), which corresponds to about 55% of the lineage-defined precursors being included by the threshold. Lines with different shades of grey correspond to preparation of CAR T cells with TCRs that eventually track to effector clusters at post-infusion timepoints, to all other CD8 T cells in the preparation of CAR T cells for which there was no evidence of TCR lineages tracking to effector clusters (note that line corresponding to all other CD8 T cells in the preparation of CAR T cells for which there was no evidence of TCR lineages tracking to effector clusters crosses the 0.4 threshold, so it can be inferred that those are cytotoxic effector precursors). FIG. 11B shows the related histogram evidencing the number of cells.

FIGS. 12A-12B show that flow cytometry data validates transcriptional characterization of CD8+ CAR T cells. FIG. 12A: Box plots comparing the percent of IFNγ-producing CD8+ CAR T cells (top) and TOX-producing CD8+ CAR T cells (bottom) between effector precursor pre-infusion cells (Tigit⁺, CD62L^{lo}, CD27⁻; left) and pre-infusion cells with the opposing surface phenotype (Tigit⁻, CD62L^{hi}, CD27+; right) co-cultured either with CD19 KO tumor (left) or CD19+ tumor (right). Dashed lines link subsets from the same patient and sample. FIG. 12B: Representative flow cytometry data visualizing GzmB-BV421 and GzmK-FITC staining of pre-infusion CD8+ CAR T cells (left) stimulated with either CD19+ tumor or CD19 KO tumor and unstimulated post-infusion CD8+ CAR T cells (right plot).

FIGS. 13A-13B show flow cytometry gating strategies for surface phenotyping and intracellular cytokine staining of SJCAR19 GMP and post-infusion samples. All samples were gated on lymphocyte-sized, single, live, CD3⁺, CD4⁻, CD8⁺, CAR⁺ cells. FIG. 13A: Gating strategy applied to GMP (pre-infusion) samples to evaluate IFNγ production from the CD8⁺CAR⁺ signature (TIGIT⁺, CD62L^{lo}, CD27⁻) and CD8⁺CAR⁺ anti-signature (TIGIT⁻, CD62L^{lo}, ⁺CD27⁺) lymphocytes. FIG. 13B: Gating strategy applied to post-infusion samples to assess frequencies of TOX and IFNγ-producing cells across CD8⁺ CAR T cells with differential staining of GzmB and GzmK. IFNγ (interferon gamma); TIGIT (T Cell Immunoreceptor with Ig and ITIM Domains); GzmB (granzyme B); GzmK (granzyme K); TOX (Thymocyte Selection Associated High Mobility Group Box); CCR5 (C—C Motif Chemokine Receptor 5); CX3CR1 (C-X3-C Motif Chemokine Receptor 1). Gating strategy prepared using FlowJo v10.7.1 software (TreeStar).

FIGS. 14A-14B show flow cytometry data from validation patient cohort. FIG. 14A: Box plots comparing the percent of IFNγ-producing CD8+ CAR T cells (top) and TOX-producing CD8+ CAR T cells (bottom) between effector precursor pre-infusion cells (Tigit⁺, CD62L^{lo}, CD27⁻; left box) and pre-infusion cells with the opposing surface phenotype (Tigit⁻, CD62L^{hi}, CD27⁺; right box) co-cultured either with CD19 KO tumor (left portion of graph) or CD19+ tumor (right portion of graph). Dashed lines pair individual patients across samples. Data is from GMP samples of patients not included in the initial transcriptional profiling dataset (patients 16-23). FIG. 14B: Box plots comparing the percent of GZMB+GZMK+(top left), GZMB+ GZMK− (top right), GZMB− GZMK+(bottom left), and GZMB-GZMK-(bottom right) CD8+ CAR T cells across pre-infusion CAR T cells co-cultured with either CD19KO or CD19+ tumor and unstimulated post-infusion CAR T cells. Patients included in analysis are 16, 17, 22, and 23 from the validation cohort.

FIGS. 15A-15B show that post-infusion peripheral blood and bone marrow CAR T cells have comparable transcriptional signatures. FIG. 15A: UMAP of week 4 and month 4 post-infusion CAR T cells colored by sample type; bone marrow is colored in pink and peripheral blood is colored in blue. FIG. 15B: Violin plots comparing gene expression across peripheral blood (PB) and bone marrow (BM) CAR T cells from each functional group.

FIG. 16 shows that clusters 3 and 8 exhibit distinct transcription factor regulatory networks. UMAP of downsampled CAR T cells clustered by differences in transcription factor regulons wherein cluster 3 cells, cluster 8 cells, and all other clusters are indicated by different shades of grey.

FIG. 17 shows that the distinct transcriptional signature between GMP effector precursors and non-precursors is not driven by differences in CAR expression. Violin plots comparing CAR transcript expression level between effector precursors and non-precursors in the GMP. Adjusted p-value=1.

FIG. 18 shows a summary of repetitive iterations of effector precursor classifier performance. Box plot summarizing the distributions of both the accuracy and the AUC across all 1,000 iterations. The box plot was graphed to show the median, upper and lower hinges (25th and 75th percentiles), two whiskers (1.5*IQR), and all outlier points (points>1.5*IQR).

FIG. 19 illustrates a block diagram of an embodiment of a computing device according to aspects of the present invention.

FIG. 20 illustrates a block diagram of an embodiment of a computing system according to aspects of the present invention.

FIG. 21 is a flow diagram of a computational method for providing a pre-effector gene signature according to aspects of the present invention.

FIG. 22 is a flow diagram of a computational method for identifying single cell gene expression data of cytotoxic effector CAR T cells according to aspects of the present invention.

FIGS. 23A and 23B are flow diagrams of computational methods for determining one or more functional effector cluster(s) associated with a functional effector T cell functional state according to aspects of the present invention.

FIG. 24 is a flow diagram of a computational method for identifying a pre-effector gene signature according to aspects of the present invention.

FIG. 25 is a flow diagram of a computational method for providing a cytotoxic effector potential according to aspects of the present invention.

FIG. 26 is a flow diagram of a computational method for calculating a gene set module score according to aspects of the present invention.

FIG. 27 is a flow diagram of a computational method for calculating gene set module score according to aspects of the present invention.

### DETAILED DESCRIPTION

This invention is based, at least in part, on the discovery that a pre-effector gene signature can be used for determining the cytotoxic effector potential of a preparation of chimeric antigen receptor (CAR) T cells. The pre-effector gene signature can be identified using single-cell gene expression. The pre-effector gene signature of the present disclosure based on single-cell gene expression provides a detailed look into complex transcriptional differences across distinct cell types and transcriptional subsets within a sample, which in bulk approaches are considered homogenous. This analysis can be used to assess, for example, CAR-transduced T cells (i.e., CAR T cells). The present disclosure provides methods for identifying transcriptionally distinct subsets of CAR T cells in a preparation of CAR T cells.

### Definitions

The term “chimeric antigen receptor” or “CAR” as used herein is defined as a cell-surface receptor comprising an extracellular target-binding domain, a transmembrane domain, and a cytoplasmic domain, comprising a lymphocyte activation domain and optionally at least one co-stimulatory signaling domain, all in a combination that is not naturally found together on a single protein. This particularly includes receptors wherein the extracellular domain and the cytoplasmic domain are not naturally found together on a single receptor protein. The chimeric antigen receptors of the present invention can be used with lymphocytes such as T cells and natural killer (NK) cells.

The term “pre-effector gene signature” as used herein is defined as a set of differentially expressed gene markers which are expressed at different levels in CAR T cells which differentiate or are more likely to differentiate into cytotoxic effector cells upon administration to a subject. In certain embodiments, the pre-effector gene signature is defined as a set of differentially expressed gene markers which are expressed at different levels in CAR T cells which differentiate or are more likely to differentiate into cytotoxic effector cells upon administration to a subject as compared to the other CAR T cells in the preparation; and/or compared to the CAR T cells which do not differentiate or are less likely to differentiate into cytotoxic effector cells upon administration to a subject; and/or compared to the CAR T cells for which no evidence of lineage-specific differentiation into cytotoxic effector CAR T cells exists upon administration to a subject. In certain embodiments, the pre-effector gene signature is CAR T cell universal. In certain embodiments, the pre-effector gene signature is CAR specific. In certain embodiments, the pre-effector gene signature is CAR generation specific (e.g., first-, second-, third-, fourth-, or fifth-generation CAR). In certain embodiments, the pre-effector gene signature is CAR antigen specific (e.g., CD19-CAR or CD123-CAR). In certain embodiments, the pre-effector gene signature is CAR stimulatory and/or co-stimulatory domain specific (e.g., CD3 or 4-1B). The pre-effector gene signature allows interrogation of the status of a preparation of CAR T cells by simultaneously considering hundreds of genes and, thereby, allows identification of subset of CAR T cells in the preparation of CAR T cells that possess cytotoxic effector potential. In certain aspects, the pre-effector gene signature provides a tool to identify the cytotoxic effector potential of a preparation of CAR T cells. In certain aspects, the pre-effector gene signature provides a tool to identify patients whose own CAR T cells can be used for immunotherapy. In certain aspects, the pre-effector gene signature provides a tool to identify subsets of effective CAR T cells for immunotherapy. In certain embodiments, the preparation of CAR T cells can be enriched to include only the identified subsets of effective CAR T cells. In certain embodiments, subsets or individual cells can be identified that match a particular threshold. In certain embodiments, a single-cell threshold is determined by one of several approaches: 1) identifying TCR lineages that link the CAR T cells to post-infusion effector clusters (cell is/is not precursor based on TCR only); 2) by using a classifier to predict whether a CAR T cell is a precursor or not (e.g., yes/no or probabilities based on machine learning); 3) enriching for all CAR T cells that go in the same direction as a set of set of differentially expressed markers (e.g., enriching for all TIGIT+ SELL-low, CD27negative CD7 CAR+ cells to get a subset that should be enriched for precursors based on a set of differentially expressed genes); and/or 4) using a set of differentially expressed genes to form a module score (e.g., a subset of 20 genes most upregulated in the precursor effectors).

A “module score” takes a set of genes and tells how high or low each CAR T cell expresses them relative to other, randomly selected genes. As such, each CAR T cell gets a score that essentially instructs that “these genes are expressed the average amount compared to the entire population”, or “these genes are expressed more than the average cell in the population”, etc., but encoded numerically in a way that represents how far above or below average they are. Next the TCR-defined cytotoxic effector precursors are examined, i.e., those CAR T cells known with certainty based on the TCR data that they are going to end up in effector clusters post-infusion, and determine what their module scores are. Any other CAR T cell with a module score similar to those of known cytotoxic effector precursors is a putative cytotoxic effector precursor. By this method subsets of putative precursors are based on the population dynamics with reference to expression values of known effector precursors (i.e., known based on TCR lineages). The threshold might be placed, by way of example and not limitation, at a place where ≥50% of your known cytotoxic effector precursors meet the thresholds. In other instances, the threshold may be set higher or lower.

As used herein, “cytotoxic effector potential” refers to the ability of CAR T cells to activate cytotoxic effector functions resulting in an effective and/or persistent clinical response. In certain embodiments, the cytotoxic effector potential is a numerical quantification or score of the likelihood that the CAR T cell would give rise to a lineage that results in post-infusion CAR T cells having an effective and/or persistent clinical response. In certain embodiments, the numerical quantification or score is obtained from data from the pre-infusion and/or post-infusion CAR T cells. In certain embodiments, the numerical quantification or score of a single CAR T cell is based on expression of marker genes within that single CAR T cell, the marker genes being derived from a mathematical analysis of single cell gene expression of the pre-infusion and post-infusion CAR T cells. In certain embodiments, the numerical quantification or score is expressed as a probability (i.e., within a range of 0 to 1).

As used herein, a “cytotoxic effector precursor” can be a CAR T cell that when administered gives rise to a CAR T cell and lineages thereof that results in functional effector post-infusion T cells.

The term “cytotoxic effector function” refers to a specialized function of a cell. Cytotoxic effector function of a T cell, for example, may be cytolytic activity (e.g., tumor killing activity) or helper activity including the secretion of cytokines.

The term “cytotoxic effector CART cells” refers to CART cells that exhibit cytotoxic effector functions resulting in an effective and/or persistent clinical response following administration to the subject.

The term “clonal TCR sequencing data” refers to clonal TCR sequencing data associated with single cell gene expression data of the preparation of CAR T cell gene expression dataset, and is clonally related to endogenous TCR sequencing data associated with each of the single cell gene expression data of cytotoxic effector CAR T cells.

The term “clonally related” refers to CAR T cells comprising at least one matching alpha and/or one matching beta TCR chain. In certain embodiments, “clonally related” refers to CAR T cells comprising at least one matching alpha. TCR chain. In certain embodiments, “clonally related” refers to CAR T cells comprising at least one matching beta TCR chain. The complementarity determining region 3 (CDR3) of each alpha and beta chain can be sequenced to determine alleles of each chain. Cells with a given TCR appearing in more than one timepoint are referred to as “lineages”.

The terms “T cell” and “T lymphocyte” are interchangeable and used synonymously herein. As used herein, T cell includes thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4⁺ T cell, a cytotoxic T cell (CTL; CD8⁺ T cell), a tumor infiltrating cytotoxic T cell (TIL; CD8⁺ T cell), CD4⁺CD8⁺ T cell, or any other subset of T cells. In certain embodiments, the T cells are CD8⁺ T cells and/or CD4⁺ T cells.

The term “effective” applied to dose or amount refers to that quantity of a CAR T cell preparation that is sufficient to result in a desired activity upon administration to a subject in need thereof. The exact amount required will vary from subject to subject, depending on the species, age, and general condition of the subject, the severity of the condition being treated, the particular drug or drugs employed, the mode of administration, and the like.

The terms “persistent”, “persist”, or “persistence” when used in relation to an immune cell refer to the ability of the immune cell (and/or its progenies) to be maintained in a recipient (e.g., a subject) for a period of time. The terms used herein encompass both in vivo and in vitro immune cell persistence.

The terms “express” and “expression” mean allowing or causing the information in a gene or DNA sequence to become produced, for example producing an RNA or a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular, or transmembrane. Expressed genes may include genes that are transcribed into messenger RNA (mRNA) and then transcribed into protein, as well as genes that are transcribed into RNA, such as transfer and ribosomal RNAs, but not translated into protein.

The term “cancer” refers to a tumor of potentially unlimited growth that expands locally by invasion and systemically by metastasis. The term “tumor” refers to a benign or malignant abnormal growth of tissue.

The terms “expand” or “expansion” when used in relation to an immune cell refer to the ability of the immune cell to undergo cellular proliferation (i.e., to increase the number of cells). The terms used herein encompass both in vivo and in vitro immune cell expansion.

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The terms “component”, “engine”, “module”, “system”, “server”, “processor”, “memory”, and the like are intended to include one or more computer-related units, such as but not limited to hardware, firmware, a combination of hardware and software, software, or software in execution. For example, a component may be, but is not limited to being, a process running on a processor, an object, an executable, a thread of execution, a program, and/or a computer. By way of illustration, both an application running on a computing device and the computing device can be a component. One or more components can reside within a process and/or thread of execution and a component may be localized on one computer and/or distributed between two or more computers. In addition, these components can execute from various computer readable media having various data structures stored thereon. The components may communicate by way of local and/or remote processes such as in accordance with a signal having one or more data packets, such as data from one component interacting with another component in a local system, distributed system, and/or across a network such as the Internet with other systems by way of the signal

The term “clustering algorithm” as used herein includes software based algorithms to perform cluster analysis. A clustering algorithm is configured to group a set of objects in such a way that objects in the same group (called a cluster) are more similar (in some sense) to each other than to those in other groups (clusters).

The term “dimensionality reduction algorithm” as used herein includes software based algorithms to perform dimensionality reduction (also referred to as dimension reduction). A dimensionality reduction algorithm is configured to transformation data from a high-dimensional space into a low-dimensional space so that the low-dimensional representation retains some meaningful properties of the original data. Some dimensionally reduction algorithms can be utilized as a clustering algorithm by selecting appropriate parameters for dimensionality reduction.

The term “machine learning algorithm” as used herein includes artificial intelligence software based computer algorithms that self-build instructions based on training data. The machine learning algorithm, once trained, can make predictions or decisions without being explicitly programmed to do so.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

If aspects of the disclosure are described as “comprising”, or versions thereof (e.g., comprises), a feature, embodiments also are contemplated “consisting of” or “consisting essentially of” the feature.

The practice of the present disclosure employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437.

The technology illustratively described herein suitably may be practiced in the absence of any element(s) not specifically disclosed herein.

The terms and expressions which have been employed are used as terms of description and not of limitation, and use of such terms and expressions do not exclude any equivalents of the features shown and described or portions thereof, and various modifications are possible within the scope of the technology claimed.

### Pre-Effector Gene Signature

The present disclosure provides, among other things, a method for generating a pre-effector gene signature for determining the cytotoxic effector potential of a preparation of chimeric antigen receptor (CAR) T cells. In some embodiments, the method comprising: a) measuring single-cell gene expression data and endogenous T cell receptor (TCR) sequencing data of the preparation of CAR T cells; b) administering the preparation of CAR T cells to a subject; c) isolating post-infusion CAR T cells at one or more time points after the administration step b); d) acquiring single-cell gene expression data and endogenous TCR sequencing data of the CAR T cells isolated in step c); e) using the endogenous TCR sequencing data obtained in steps a) and d) to determine which CAR T cells from the preparation of CAR T cells are cytotoxic effector precursors by determining which of the CAR T cells of the preparation of CAR T cells share a clonally related TCR with post-infusion CAR T cells that differentiated into cytotoxic effector CAR T cells; and f) generating a pre-effector gene signature by determining which set of genes are differentially expressed in the cytotoxic effector precursors as compared to other or all other CAR T cells from the preparation.

In certain embodiments, the pre-effector gene signature is defined as a set of differentially expressed gene markers which are expressed at different levels in CAR T cells which differentiate or are more likely to differentiate into cytotoxic effector cells upon administration to a subject. In certain embodiments, the pre-effector gene signature is defined as a set of differentially expressed gene markers which are expressed at different levels in CAR T cells which differentiate or are more likely to differentiate into cytotoxic effector cells upon administration to a subject as compared to the other CAR T cells in the preparation; and/or compared to the CAR T cells which do not differentiate or are less likely to differentiate into cytotoxic effector cells upon administration to a subject; and/or compared to the CAR T cells for which no evidence of lineage-specific differentiation into cytotoxic effector CAR T cells exists upon administration to a subject. In certain embodiments, the lack of evidence is based on the inability to identify linked TCRs between the preparation of CAR T cells and the post-infusion infector CAR T cell cluster.

In certain embodiments, a CAR T cell is determined to be a CAR T cell that does not differentiate into a cytotoxic effector CAR T cell and/or a CAR T cell for which no evidence of lineage-specific differentiation into cytotoxic effector CAR T cells exists can be measured within a certain period after administration to the subject. In certain embodiments, the time period is at about 1-25 weeks, about 1-20 weeks, about 1-18 weeks, about 1-16 weeks, about 1-12 weeks, about 1-10 weeks, about 1-8 weeks, about 1-6 weeks, about 1-5 weeks, about 1-4 weeks, about 1-3 weeks, or about 1-2 weeks post-infusion. In certain embodiments, the time period is at about 1-3 weeks post-infusion. In certain embodiments, the time period is at about 2-3 weeks post-infusion. In certain embodiments, the time period is at 1 week post-infusion. In certain embodiments, the time period is at 2 weeks post-infusion. In certain embodiments, the time period is at 3 weeks post-infusion. In certain embodiments, the time period is at 4 weeks post-infusion. In certain embodiments, the time period is at 5 weeks post-infusion. In certain embodiments, the time period is at 8 weeks post-infusion. In certain embodiments, the time period is at 3 months post-infusion. In certain embodiments, the time period is at 6 months post-infusion. In certain embodiments, the time period is between 1-3 weeks post-infusion.

In certain embodiments, the pre-effector gene signature is universal. In certain embodiments, the pre-effector gene signature is CAR specific. In certain embodiments, the pre-effector gene signature is CAR generation specific. In certain embodiments, the pre-effector gene signature is CAR antigen specific (e.g., CD19-CAR or CD123-CAR). In certain embodiments, the pre-effector gene signature is CAR stimulatory and/or co-stimulatory domain specific (e.g., CD3ζ or 4-1BB).

The pre-effector gene signature provides the following competitive advantages relative to existing technology: 1) single-cell analysis, as used herein, provides a more detailed look into complex transcriptional differences across distinct CAR T cell types and transcriptional subsets within a sample of CAR T cells, whereas existing in-bulk approaches are limited by homogeneity (i.e., the ability to identify much of the cell-cell heterogeneity is lost when RNA is isolated and sequenced in bulk from a whole CAR T cell product); 2) use of the endogenous TCR as a barcode to track CAR T cell lineages, in combination with single-cell gene expression, provides the ability to identify unique signatures of CAR T cells with cytotoxic effector potential by tracing the TCR from effective CAR T cells back to those in the pre-infusion CAR T cell product; 3) provides information associated with effector CAR T cells that are different from profiles provided by existing technologies (e.g., while previous technologies have found that higher percentages of CD27+ cells were associated with complete and partial responses in the patient, it was identified with the present pre-effector gene signature that a decrease in CD27 was associated with increased effector potential; similarly, presence of an inhibitory receptor, TIGIT, was surprisingly found to be correlated with potent effector potential by using the present pre-effector gene signature); and 4) expands upon the currently known gene sets and markers associated with CAR T effector functionality.

In certain embodiments, the methods of the present disclosure can be used to assess the cytotoxic effector potential of a CAR T cell preparation to be infused into a subject in need thereof. In certain embodiments, the cytotoxic effector potential of a CAR T cell preparation can be measured by comparing a CAR T cell preparation single-cell gene expression data, single-cell surface marker abundance data, (e.g., via flow cytometry, CiteSeq, or splint oligos), or a set of differentially expressed markers with the gene signature associated with precursor CAR T cells that develop potent effector functions following administration.

In certain embodiments, the differentially expressed markers comprise one or more marker genes selected from PLEK, TIGIT, EOMES, ELOVL4, MSC, GZMH, AC017002.3, KLRD1, CCL4, LINC01943, CCL3, GZMK, HLA-DQA1, HLA-DPA1, HLA-DRB1, CD52, MAL, HLA-DRB5, LTB, CD74, CAPG, SH3BGRL3, IFITM3, HLA-DQA2, HLA-DPB1, FLT3LG, NPM1, HLA-DRA, CD86, CD7, PPP2R2B, IFNG, CD27, RPL12, TIMP1, FABP5, HSPD1, NOSIP, F2R, GSTP1, LIMID2, RPLP0, IFITM1, SPOCK2, HLA-DQB1, ITM2B, TPT1, IFITM2, RPL8, SEC11C, EVL, LRRN3, SUB1, ITM2C, CD8B, CST7, HSP90AB1, RPL5, PARK7, GPSM3, SELL, CD3E, GALM, RPS27, GAPDH, DRAP1, RPS7, PPIA, CENPU, S100A4, RPL7, VSIR, TMSB10, STK17B, BCAT1, EEF2, UCP2, B2M, OSTF1, PIM1, RNF213, INSIG1, OASL, OAZ1, FAM3C, IER2, PPA1, CTH, FYN, RPS6, LYAR, PLAC8, RPL3, RGS1, EMP3, TMBIM6, RPL7A, FXYD5, RAN, PRDX4, IMPDH2, CD44, LEF1, HLA-DMA, LAPTM5, ASB2, GNLY, RGS10, AHCY, LAG3, CISH, GLIPR2, BAX, MYL12A, CD70, CCT8, PCBP2, ACTA2, CSTB, HLA-E, MFSD10, AES, PDIA6, SUSD3, RPS5, PGAM1, XBP1, PTGER2, FAM216A, IL2RA, CACYBP, RPSA, LAT, BPGM, SLC25A3, EIF1AY, CD3D, RPS3A, MCUB, MTHFD2, SLC43A3, NUCB2, ATP6V1G1, SHMT2, AMZ2, HLA-B, SKAP2, CDC25B, PTTG1, LMAN1, RPS4Y1, CALM1, LCK, H2AFV, MDH2, PAICS, ASNS, ALOX5AP, H3F3A, PRDX3, SLF1, MALAT1, CD55, SYNE2, H3F3B, MT-ND4, GSTK1, IL7R, CCDC107, SFT2D1, FBL, FCER1G, CDKN2D, CD69, TXNIP, MT-ND4L, RPS18, S100A6, ZNF683, LGALS3, EIF4EBP1, RPL6, ACAT1, AQP3, SMS, LYRM4, IL32, RIPOR2, RPL36, S100A10, GLIPR1, ITGB7, IDH2, PDE6G, PLP2, CDC42SE1, CTNNAL1, VDAC1, MUC20-OT1, CD82, MRPL10, BIN2, AK2, CCT4, ISG20, EID1, BTG2, DEF6, ATP5F1E, LIMS2, LEPROTL1, DDAH2, GIMAP7, LST1, CD300A, PCBD1, RPL4, MT-CYB, SH2D2A, ISG15, ARL3, CCND3, CD3G, PEBP1, TRABD2A, BST2, UNG, SRI, SNAP47, METTL9, GINS2, DOK2, RHOF, C9orf16, HNRNPC, FAH, GPI, TMIGD2, PTPN6, AP3S1, S100A11, RPL22L1, CD8B2, R3HDM4, SOCS2, C20orf27, MRPL11, HLA-A, FARSB, H2AFZ, PLA2G16, NPDC1, GARS, SIT1, POLR2L, PCK2, LYRM1, IL16, RILPL2, CNN2, CMC1, TRBC2, SOD1, WARS, GYG1, NDUFV2, SARAF, ARF6, LDHA, RASA3, PRKX, TXN, VAMP8, OXCT1, TNFSF10, CSK, PRELID1, HAPLN3, EIF4H, SP140, ILK, CYFIP2, POLE4, ATIC, OTULINL, DBN1, IDS, RFLNB, PYCARD, ZFP36L1, TPI1, DHRS3, PTGES3, RGS19, MGAT4A, TRAC, SNRPC, LGALS3BP, PAXX, MRPL37, CCDC69, LY6E, DESI1, NMRAL1, TGFB1, CORO1B, CYC1, TESC, PHGDH, P2RX4, SLC1A5, TRBC1, NUDT21, HSPA9, RGS14, CXCR3, LSP1, HTATSF1, MYDGF, MDH1, NCR3, JAK1, FYB1, FDPS, CD248, PSMG1, STAP1, CYSTM1, TMEM123, MAD2L1, H1FX, ARL4C, UBALD2, OXNAD1, TK1, SERINC5, RHOH, RUVBL2, PARP1, MT-ND5, CCDC85B, NDUFA12, SURF4, DNAJB6, ETFA, SNRPA, GGH, CASP3, PARVG, CD47, BIN1, ANXA11, TMEM173, UBL5, MZT2B, HLA-C, CD247, RSL24D1, TYMS, ID3, ELOVL5, ARHGAP45, HSPE1, C9orf78, XRCC6, TCP1, ACAT2, SATB1, CYBA, DUT, LTA4H, HCST, IRF1, MSN, CDCA7, IL9R, CCL5, LAIR1, BHLHE40, ZFP36L2, FAM162A, VAMP5, LAT2, RCAN3, BNIP3, PNPLA2, SAMIHD1, CD8A, PIM2, ARHGEF1, CALHM2, GPX4, SNAI3, ILORA, H2AFJ, CCDC28B, PCBP1, KLF2, ACAP2, ZNF580, TXK, RBMS1, ABLIM1, ETHE1, SLFN5, PECAM1, C12orf57, HSP90AA1, CCT3, IL17RA, SPINT2, LGALS1, CREM, MAD1L1, NINJ1, WDR54, ESYT1, IDI1, FAAP20, ALDOA, STK4, CD79B, TMEM160, AC004687.1, PRMT2, LAMTOR4, SMCHD1, IKZF1, SSB, TMEM204, SOCS1, MZT2A, EPSTI1, NBDY, CCT2, FOXP1, MFNG, CYTIP, ANKRD37, CD9, NDFIP1, LITAF, CAMK2G, KLF6, KDM5B, CDKN1B, ATP5F1D, PPID, PGK1, EGLN3, RASGRP2, PLPP1, LIME1, Clorfl62, HISTIHID, ATP6VOE2, ZYX, ZAP70, OSM, JAML, SELPLG, CAMK4, PTPRCAP, LCP2, DGKA, TRIM22, GADD45B, SYNRG, UBE2L6, RHEB, NFKBIA, PDLIM2, AGTRAP, ACTN1, MOSPD3, BTG1, MYH9, CCT5, PSAT1, PRDX1, TLN1, NDUFA3, SYTL1, CCM2, ARHGAP4, ETS1, PRKCB, PNRC1, RASSF1, TPST2, NKG7, DDX17, XIST, COTL1, SUN2, CDKN1A, KDELR2, HBEGF, CD40LG, MMP25-AS1, MT1E, ERO1A, IL2RG, PTGER4, PDE4D, EIF4A3, TAP1, JUNB, ANXA1, GNB2, GNAS, RPS26, TOP2A, MTRNR2L8, MT-ND6, MT1X, CENPF, TSC22D3, GLUL, NEAT1, TRAV8-2, STAT1, HIST1H2AL, HIST1H3B, TOX and combinations thereof.

In certain embodiments, the expression of the markers is increased compared to all other CAR T cells in the CAR T cell preparation. In certain embodiments, the expression of the markers is increased compared to the expression on CAR T cells that do not differentiate into cytotoxic effector CAR T cells. In certain embodiments, the expression of the markers is increased compared to the expression on CAR T cells that did not differentiate into cytotoxic effector CAR T cells. In certain embodiments, the expression of the markers is increased compared to the expression on CAR T cells that are less likely to differentiate into cytotoxic effector CAR T cells. In certain embodiments, the expression of the markers is increased compared to the expression on CAR T cells for which no evidence of lineage-specific differentiation into cytotoxic effector CAR T cells exists.

In certain embodiments, the expression of the markers is decreased compared to the expression of all other CAR T cells in the preparation. In certain embodiments, the expression of the markers is decreased compared to the expression on CAR T cells that do not differentiated into cytotoxic effector CAR T cells. In certain embodiments, the expression of the markers is decreased compared to the expression on CAR T cells that did not differentiate into cytotoxic effector CAR T cells. In certain embodiments, the expression of the markers is decreased compared to the expression on CAR T cells that are less likely to differentiate into cytotoxic effector CAR T cells. In certain embodiments, the expression of the markers is decreased compared to the expression on CAR T cells for which no evidence of lineage-specific differentiation into cytotoxic effector CAR T cells exists.

In certain embodiments, the expression of a set of specific markers is increased and the expression of a different set of specific markers is decreased.

In certain embodiments, the pre-effector gene signature or set of differentially expressed markers comprise one or more marker genes selected from the genes listed in Tables 1 and/or 2. In certain embodiments, the pre-effector gene signature or set of differentially expressed markers comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 300, at least 350, at least 400, at least 450, at least 500 marker genes selected from the genes listed in Tables 1 and/or 2.

TABLE 1
Genes with higher expression in cytotoxic
effector precursor CAR T cells

| | | | Proportion | |
|---|---|---|---|---|
| | | | of all other | |
| | | Proportion | CD8+ CAR T | |
| | | of | cells in the | |
| | | cytotoxic | preparation | |
| | | effector | of CAR T | |
| | Average | precursors | cells | |
| | log2 fold | expressing | expressing | Adjusted |
| Gene | change | the gene | the gene | p-value |
| PLEK | 0.364542 | 0.292 | 0.016 | 6E−292 |
| TIGIT | 0.68609 | 0.48 | 0.06 | 4.5E−208 |
| EOMES | 0.217757 | 0.226 | 0.016 | 2.2E−179 |
| ELOVL4 | 0.254744 | 0.313 | 0.031 | 1E−171 |
| MSC | 0.480344 | 0.439 | 0.067 | 2.4E−150 |
| GZMH | 0.552716 | 0.345 | 0.043 | 1.3E−148 |
| AC017002.3 | 0.524068 | 0.464 | 0.079 | 2.9E−140 |
| KLRD1 | 0.344266 | 0.27 | 0.029 | 8.1E−135 |
| CCL4 | 1.045816 | 0.295 | 0.037 | 8.5E−122 |
| LINC01943 | 1.088062 | 0.668 | 0.199 | 3.5E−118 |
| CCL3 | 0.707193 | 0.266 | 0.033 | 1.3E−111 |
| GZMK | 1.222264 | 0.48 | 0.103 | 1.6E−109 |
| HLA-DQA1 | 1.127124 | 0.624 | 0.197 | 6.77E−93 |
| HLA-DPA1 | 1.523774 | 0.934 | 0.664 | 6.08E−88 |
| HLA-DRB1 | 1.318461 | 0.987 | 0.827 | 7.48E−85 |
| HLA-DRB5 | 1.084358 | 0.972 | 0.666 | 1.89E−76 |
| CD74 | 0.759303 | 1 | 0.999 | 7.78E−61 |
| SH3BGRL3 | −0.73758 | 1 | 0.998 | 4.65E−60 |
| HLA-DQA2 | 0.762851 | 0.552 | 0.188 | 1.28E−59 |
| HLA-DPB1 | 0.897488 | 0.934 | 0.658 | 3.17E−56 |
| NPM1 | 0.607037 | 1 | 0.993 | 4.43E−55 |
| HLA-DRA | 0.901164 | 0.978 | 0.832 | 7.61E−55 |
| CD86 | 0.284958 | 0.448 | 0.136 | 3.57E−54 |
| PPP2R2B | 0.25164 | 0.31 | 0.075 | 1.97E−52 |
| IFNG | 0.494949 | 0.527 | 0.193 | 5.05E−47 |
| RPL12 | 0.414022 | 1 | 0.998 | 3.08E−45 |
| FABP5 | 0.772625 | 0.934 | 0.737 | 9.2E−43 |
| HSPD1 | 0.683846 | 0.978 | 0.921 | 4.58E−42 |
| F2R | 0.38164 | 0.574 | 0.238 | 8.09E−41 |
| GSTP1 | 0.56311 | 1 | 0.961 | 1.13E−40 |
| RPLPO | 0.345877 | 1 | 0.999 | 5.33E−40 |
| HLA-DQB1 | 0.808195 | 0.824 | 0.548 | 8.36E−39 |
| TPT1 | −0.34194 | 1 | 0.999 | 1.32E−37 |
| RPL8 | 0.283226 | 1 | 0.999 | 2.34E−36 |
| SEC11C | 0.573464 | 0.865 | 0.63 | 2.44E−36 |
| SUB1 | 0.43444 | 0.997 | 0.982 | 3.27E−33 |
| CD8B | −0.57319 | 0.972 | 0.968 | 1.36E−32 |
| CST7 | 0.840627 | 0.994 | 0.944 | 2.11E−32 |
| HSP90AB1 | 0.786892 | 0.987 | 0.955 | 4.25E−32 |
| RPL5 | 0.373615 | 1 | 0.996 | 1.73E−31 |
| PARK7 | 0.366106 | 0.994 | 0.977 | 2.14E−31 |
| CD3E | −0.36422 | 1 | 0.997 | 2.4E−29 |
| GALM | 0.491303 | 0.815 | 0.56 | 5.83E−29 |
| RPS27 | −0.37725 | 1 | 0.998 | 4.17E−28 |
| RPS7 | 0.252076 | 1 | 0.998 | 4.58E−27 |
| PPIA | 0.248034 | 1 | 0.998 | 6.2E−27 |
| CENPU | 0.432574 | 0.875 | 0.651 | 2.19E−26 |
| S100A4 | −0.58301 | 0.997 | 0.996 | 2.79E−26 |
| RPL7 | 0.364734 | 1 | 0.994 | 3.85E−26 |
| BCAT1 | 0.290221 | 0.574 | 0.278 | 1.18E−25 |
| EEF2 | 0.344809 | 0.997 | 0.996 | 1.24E−25 |
| UCP2 | −0.54324 | 0.966 | 0.965 | 1.91E−25 |
| OSTF1 | 0.423216 | 0.978 | 0.953 | 4.57E−25 |
| RNF213 | 0.534216 | 0.925 | 0.823 | 6.06E−24 |
| INSIG1 | 0.627948 | 0.893 | 0.701 | 7.57E−24 |
| OASL | 0.275431 | 0.549 | 0.272 | 1.26E−23 |
| OAZ1 | 0.305905 | 1 | 0.994 | 5.64E−23 |
| FAM3C | 0.227502 | 0.458 | 0.208 | 7.1E−23 |
| PPA1 | 0.388347 | 0.984 | 0.909 | 7.75E−23 |
| CTH | 0.236701 | 0.288 | 0.104 | 1.08E−22 |
| FYN | 0.335035 | 0.467 | 0.225 | 1.98E−22 |
| RPS6 | 0.241621 | 1 | 0.999 | 3.14E−22 |
| LYAR | 0.394531 | 0.715 | 0.438 | 3.52E−22 |
| RPL3 | 0.259626 | 1 | 0.999 | 4.38E−22 |
| RGS1 | 0.250142 | 0.295 | 0.108 | 5.48E−22 |
| EMP3 | −0.3725 | 0.997 | 0.994 | 5.92E−22 |
| TMBIM6 | 0.338155 | 0.997 | 0.981 | 6.57E−22 |
| RPL7A | 0.243024 | 1 | 0.999 | 1.29E−21 |
| RAN | 0.339958 | 0.991 | 0.987 | 2.62E−21 |
| PRDX4 | 0.347073 | 0.759 | 0.499 | 2.79E−21 |
| IMPDH2 | 0.424348 | 0.962 | 0.903 | 7.39E−21 |
| HLA-DMA | 0.425711 | 0.853 | 0.643 | 1.28E−20 |
| LAPTM5 | −0.35339 | 0.991 | 0.989 | 1.55E−20 |
| ASB2 | 0.30472 | 0.442 | 0.208 | 1.94E−20 |
| GNLY | 1.418804 | 0.245 | 0.086 | 2.28E−20 |
| AHCY | 0.391016 | 0.878 | 0.738 | 3.83E−20 |
| LAG3 | 0.428553 | 0.699 | 0.413 | 1.01E−19 |
| MYL12A | −0.31319 | 1 | 0.997 | 1.57E−19 |
| CD70 | 0.371027 | 0.749 | 0.465 | 1.9E−19 |
| CCT8 | 0.364334 | 0.984 | 0.932 | 2.89E−19 |
| PCBP2 | −0.3099 | 1 | 0.995 | 3.05E−19 |
| ACTA2 | 0.26265 | 0.379 | 0.168 | 3.74E−19 |
| CSTB | −0.45439 | 0.937 | 0.929 | 4.66E−19 |
| HLA-E | −0.39784 | 0.997 | 0.995 | 4.89E−19 |
| MFSD10 | 0.352862 | 0.859 | 0.653 | 6.77E−19 |
| PDIA6 | 0.364897 | 0.95 | 0.849 | 1.39E−18 |
| RPS5 | 0.222551 | 1 | 0.998 | 2.21E−18 |
| PGAM1 | 0.426531 | 1 | 0.977 | 2.84E−18 |
| FAM216A | 0.243517 | 0.586 | 0.319 | 4E−18 |
| IL2RA | 0.579418 | 0.85 | 0.656 | 4.2E−18 |
| CACYBP | 0.386203 | 0.975 | 0.901 | 5.03E−18 |
| RPSA | 0.204452 | 1 | 0.999 | 7.23E−18 |
| BPGM | 0.261628 | 0.382 | 0.179 | 1.29E−17 |
| SLC25A3 | 0.299645 | 0.997 | 0.982 | 1.84E−17 |
| EIF1AY | 0.3262 | 0.492 | 0.253 | 1.95E−17 |
| CD3D | −0.33727 | 1 | 0.994 | 2.28E−17 |
| RPS3A | 0.212653 | 1 | 0.999 | 5.49E−17 |
| MTHFD2 | 0.441025 | 0.947 | 0.844 | 9.49E−17 |
| SLC43A3 | 0.270568 | 0.549 | 0.304 | 9.92E−17 |
| ATP6V1G1 | −0.32276 | 0.953 | 0.948 | 1.25E−16 |
| SHMT2 | 0.338559 | 0.975 | 0.929 | 2.27E−16 |
| AMZ2 | 0.336464 | 0.871 | 0.702 | 2.41E−16 |
| SKAP2 | 0.21102 | 0.461 | 0.23 | 2.98E−16 |
| PTTG1 | 0.384121 | 0.978 | 0.863 | 7.45E−16 |
| LMAN1 | 0.367964 | 0.922 | 0.812 | 1.54E−15 |
| RPS4Y1 | 0.557601 | 0.605 | 0.332 | 1.78E−15 |
| CALM1 | −0.31236 | 1 | 0.997 | 3.77E−15 |
| H2AFV | −0.44041 | 0.94 | 0.92 | 5.43E−15 |
| MDH2 | 0.276264 | 0.972 | 0.951 | 6.28E−15 |
| PAICS | 0.385548 | 0.834 | 0.663 | 1.53E−14 |
| ASNS | 0.346132 | 0.705 | 0.478 | 1.68E−14 |
| ALOX5AP | 0.353353 | 0.997 | 0.972 | 1.74E−14 |
| H3F3A | −0.28675 | 0.997 | 0.995 | 1.85E−14 |
| PRDX3 | 0.340839 | 0.978 | 0.904 | 3.16E−14 |
| SLF1 | 0.224856 | 0.69 | 0.417 | 3.82E−14 |
| MT-ND4 | 0.351637 | 1 | 0.998 | 5.17E−14 |
| GSTK1 | 0.314541 | 0.997 | 0.981 | 5.86E−14 |
| SFT2D1 | 0.286148 | 0.755 | 0.552 | 6.73E−14 |
| FBL | 0.334288 | 0.981 | 0.893 | 7.27E−14 |
| MT-ND4L | 0.258746 | 1 | 0.999 | 3.04E−13 |
| RPS18 | 0.20424 | 1 | 0.999 | 3.33E−13 |
| S100A6 | −0.29236 | 1 | 0.998 | 3.59E−13 |
| EIF4EBP1 | 0.370191 | 0.897 | 0.753 | 3.96E−13 |
| RPL6 | 0.216228 | 1 | 0.997 | 4.45E−13 |
| ACAT1 | 0.332347 | 0.752 | 0.557 | 4.86E−13 |
| SMS | 0.308196 | 0.89 | 0.733 | 1.06E−12 |
| LYRM4 | 0.239747 | 0.602 | 0.385 | 1.54E−12 |
| IL32 | −0.3809 | 1 | 0.996 | 1.89E−12 |
| RPL36 | 0.236737 | 1 | 0.997 | 2E−12 |
| S100A10 | −0.23782 | 1 | 0.997 | 2.14E−12 |
| GLIPR1 | 0.296838 | 0.639 | 0.412 | 2.47E−12 |
| CTNNAL1 | 0.284896 | 0.762 | 0.534 | 4.96E−12 |
| VDAC1 | 0.297602 | 0.984 | 0.937 | 5.01E−12 |
| MUC20- | 0.235035 | 0.687 | 0.466 | 5.22E−12 |
| OT1 | | | | |
| MRPL10 | 0.320054 | 0.796 | 0.622 | 7.13E−12 |
| AK2 | 0.27343 | 0.903 | 0.765 | 7.89E−12 |
| CCT4 | 0.305736 | 0.947 | 0.908 | 8.37E−12 |
| EID1 | 0.273114 | 0.984 | 0.94 | 8.73E−12 |
| ATP5F1E | −0.27621 | 0.994 | 0.993 | 1.37E−11 |
| CD300A | 0.216842 | 0.467 | 0.265 | 6.85E−11 |
| PCBD1 | 0.28079 | 0.784 | 0.573 | 6.87E−11 |
| RPL4 | 0.201117 | 1 | 0.997 | 7.65E−11 |
| MT-CYB | 0.313603 | 1 | 0.999 | 1.02E−10 |
| SH2D2A | 0.334596 | 0.881 | 0.746 | 1.08E−10 |
| ARL3 | 0.252585 | 0.665 | 0.437 | 1.14E−10 |
| CCND3 | −0.31414 | 0.981 | 0.965 | 1.34E−10 |
| PEBP1 | 0.288346 | 0.956 | 0.867 | 1.8E−10 |
| BST2 | 0.263656 | 0.959 | 0.911 | 1.88E−10 |
| UNG | 0.305828 | 0.473 | 0.279 | 2.12E−10 |
| SRI | 0.242031 | 0.984 | 0.936 | 2.23E−10 |
| SNAP47 | 0.216558 | 0.646 | 0.426 | 2.47E−10 |
| GINS2 | 0.312159 | 0.777 | 0.561 | 4.27E−10 |
| RHOF | −0.29502 | 0.875 | 0.873 | 5.05E−10 |
| C9orf16 | −0.24906 | 0.984 | 0.977 | 5.08E−10 |
| HNRNPC | 0.220929 | 0.997 | 0.976 | 5.58E−10 |
| FAH | 0.23444 | 0.708 | 0.493 | 5.81E−10 |
| GPI | 0.340733 | 0.997 | 0.975 | 6.54E−10 |
| AP3S1 | 0.321655 | 0.824 | 0.667 | 1.26E−09 |
| RPL22L1 | 0.382118 | 0.875 | 0.737 | 1.75E−09 |
| SOCS2 | 0.216841 | 0.542 | 0.333 | 2.34E−09 |
| C20orf27 | 0.201628 | 0.643 | 0.424 | 2.37E−09 |
| MRPL11 | 0.264603 | 0.887 | 0.763 | 2.93E−09 |
| FARSB | 0.204357 | 0.539 | 0.333 | 3.58E−09 |
| H2AFZ | 0.299288 | 0.997 | 0.99 | 4.09E−09 |
| PLA2G16 | 0.206661 | 0.461 | 0.274 | 4.6E−09 |
| GARS | 0.346611 | 0.937 | 0.817 | 4.82E−09 |
| SIT1 | −0.29795 | 0.969 | 0.966 | 7.36E−09 |
| PCK2 | 0.25122 | 0.755 | 0.555 | 7.77E−09 |
| LYRM1 | 0.230174 | 0.618 | 0.421 | 7.84E−09 |
| RILPL2 | 0.200797 | 0.677 | 0.459 | 8.67E−09 |
| CNN2 | −0.28313 | 0.987 | 0.985 | 8.73E−09 |
| CMC1 | 0.216121 | 0.683 | 0.477 | 9.69E−09 |
| SOD1 | 0.276322 | 0.997 | 0.946 | 1.33E−08 |
| WARS | 0.275482 | 0.74 | 0.557 | 1.4E−08 |
| GYG1 | 0.277249 | 0.765 | 0.605 | 1.43E−08 |
| NDUFV2 | 0.213464 | 0.994 | 0.972 | 1.47E−08 |
| LDHA | 0.347309 | 1 | 0.991 | 2.05E−08 |
| TXN | 0.220623 | 0.994 | 0.986 | 2.46E−08 |
| OXCT1 | 0.208471 | 0.749 | 0.544 | 2.68E−08 |
| EIF4H | 0.231956 | 0.966 | 0.917 | 7E−08 |
| SP140 | 0.219686 | 0.784 | 0.587 | 7.72E−08 |
| POLE4 | 0.22148 | 0.787 | 0.583 | 9.57E−08 |
| ATIC | 0.262769 | 0.721 | 0.532 | 9.9E−08 |
| IDS | 0.215032 | 0.746 | 0.556 | 1.15E−07 |
| TP11 | 0.257448 | 1 | 0.997 | 1.56E−07 |
| PTGES3 | 0.214482 | 0.981 | 0.961 | 1.72E−07 |
| SNRPC | 0.205082 | 0.972 | 0.918 | 2.1E−07 |
| MRPL37 | 0.233422 | 0.937 | 0.864 | 2.57E−07 |
| LY6E | −0.31314 | 0.975 | 0.972 | 4.18E−07 |
| DESI1 | 0.209873 | 0.755 | 0.592 | 4.29E−07 |
| NMRAL1 | 0.206293 | 0.649 | 0.458 | 4.77E−07 |
| PHGDH | 0.316482 | 0.903 | 0.794 | 8.82E−07 |
| SLC1A5 | 0.279386 | 0.922 | 0.817 | 1.03E−06 |
| NUDT21 | 0.214346 | 0.95 | 0.922 | 1.18E−06 |
| HSPA9 | 0.217813 | 0.897 | 0.761 | 1.28E−06 |
| LSP1 | 0.257904 | 0.994 | 0.989 | 1.54E−06 |
| MYDGF | 0.232701 | 0.887 | 0.787 | 1.59E−06 |
| MDH1 | 0.227603 | 0.962 | 0.923 | 1.73E−06 |
| FYB1 | −0.29207 | 0.953 | 0.948 | 2.06E−06 |
| FDPS | 0.26779 | 0.956 | 0.902 | 2.15E−06 |
| PSMG1 | 0.208593 | 0.777 | 0.604 | 2.72E−06 |
| CYSTM1 | 0.204725 | 0.687 | 0.497 | 3.19E−06 |
| TMEM123 | −0.26894 | 0.868 | 0.863 | 3.33E−06 |
| MAD2L1 | 0.212976 | 0.931 | 0.779 | 3.36E−06 |
| TK1 | 0.226306 | 0.918 | 0.749 | 4.29E−06 |
| RUVBL2 | 0.229227 | 0.884 | 0.754 | 6.75E−06 |
| PARP1 | 0.203658 | 0.959 | 0.861 | 6.97E−06 |
| MT-ND5 | 0.232713 | 1 | 0.997 | 6.97E−06 |
| NDUFA12 | −0.23702 | 0.972 | 0.949 | 7.67E−06 |
| SURF4 | 0.221146 | 0.897 | 0.749 | 8.23E−06 |
| DNAJB6 | 0.206877 | 0.815 | 0.673 | 8.45E−06 |
| ETFA | 0.225284 | 0.862 | 0.759 | 8.86E−06 |
| SNRPA | 0.207385 | 0.966 | 0.883 | 1.15E−05 |
| GGH | 0.202945 | 0.799 | 0.623 | 1.22E−05 |
| CASP3 | 0.236041 | 0.639 | 0.471 | 1.24E−05 |
| ANXA11 | −0.21677 | 0.972 | 0.97 | 1.46E−05 |
| TMEM173 | 0.245282 | 0.743 | 0.606 | 1.48E−05 |
| UBL5 | −0.21325 | 0.972 | 0.971 | 1.55E−05 |
| HLA-C | −0.2018 | 1 | 0.999 | 2.03E−05 |
| CD247 | −0.22724 | 0.978 | 0.946 | 2.09E−05 |
| RSL24D1 | 0.212749 | 0.959 | 0.903 | 2.14E−05 |
| TYMS | −0.57504 | 0.777 | 0.758 | 2.23E−05 |
| ELOVL5 | 0.213651 | 0.906 | 0.809 | 2.47E−05 |
| HSPE1 | 0.31641 | 0.89 | 0.798 | 2.69E−05 |
| XRCC6 | 0.222897 | 0.978 | 0.937 | 3.17E−05 |
| TCP1 | 0.283909 | 0.871 | 0.785 | 3.47E−05 |
| ACAT2 | 0.236209 | 0.865 | 0.741 | 3.56E−05 |
| CYBA | −0.21669 | 0.987 | 0.985 | 3.77E−05 |
| DUT | 0.213482 | 0.981 | 0.932 | 3.96E−05 |
| LTA4H | 0.205039 | 0.824 | 0.667 | 4.23E−05 |
| HCST | −0.28666 | 0.978 | 0.969 | 4.31E−05 |
| MSN | −0.27845 | 0.928 | 0.927 | 4.68E−05 |
| CDCA7 | 0.236577 | 0.846 | 0.714 | 4.78E−05 |
| CCL5 | 0.221699 | 0.962 | 0.836 | 6.12E−05 |
| FAM162A | 0.317444 | 0.944 | 0.845 | 8.37E−05 |
| VAMP5 | −0.25885 | 0.887 | 0.883 | 9.44E−05 |
| BNIP3 | 0.390787 | 0.721 | 0.57 | 0.000122 |
| CD8A | −0.23762 | 0.987 | 0.979 | 0.000189 |
| PIM2 | 0.206091 | 0.768 | 0.597 | 0.000189 |
| ETHE1 | −0.22061 | 0.903 | 0.897 | 0.00058 |
| C12orf57 | −0.28687 | 0.918 | 0.91 | 0.000662 |
| HSP90AA1 | 0.251701 | 0.994 | 0.99 | 0.000685 |
| CCT3 | 0.201872 | 0.944 | 0.874 | 0.000713 |
| CREM | 0.217534 | 0.737 | 0.579 | 0.000788 |
| MAD1L1 | 0.221639 | 0.737 | 0.596 | 0.000802 |
| WDR54 | 0.2313 | 0.925 | 0.825 | 0.000825 |
| IDI1 | 0.209808 | 0.774 | 0.645 | 0.001176 |
| ALDOA | 0.234989 | 1 | 0.979 | 0.001289 |
| TMEM160 | −0.20384 | 0.893 | 0.878 | 0.001796 |
| LAMTOR4 | −0.20229 | 0.944 | 0.941 | 0.002737 |
| SSB | 0.212642 | 0.89 | 0.777 | 0.00352 |
| CCT2 | 0.219 | 0.897 | 0.806 | 0.006148 |
| CYTIP | 0.203661 | 0.969 | 0.949 | 0.007818 |
| ANKRD37 | 0.316329 | 0.382 | 0.262 | 0.008438 |
| NDFIP1 | −0.21053 | 0.815 | 0.813 | 0.010809 |
| LITAF | 0.226619 | 0.893 | 0.794 | 0.01413 |
| PPID | 0.300591 | 0.658 | 0.537 | 0.024128 |
| PGK1 | 0.263683 | 1 | 0.989 | 0.024259 |
| MYH9 | −0.29616 | 0.931 | 0.913 | 0.155802 |
| CCT5 | 0.222224 | 0.884 | 0.834 | 0.167334 |
| PSAT1 | 0.243103 | 0.818 | 0.709 | 0.170623 |
| PRDX1 | 0.209845 | 0.984 | 0.96 | 0.172689 |
| RASSF1 | −0.20622 | 0.831 | 0.803 | 0.564821 |
| NKG7 | 0.355257 | 0.991 | 0.981 | 1 |
| DDX17 | −0.22002 | 0.787 | 0.771 | 1 |
| CDKN1A | 0.363393 | 0.492 | 0.375 | 1 |
| MT1E | 0.428875 | 0.618 | 0.504 | 1 |
| ERO1A | 0.250975 | 0.671 | 0.592 | 1 |
| EIF4A3 | −0.21398 | 0.63 | 0.612 | 1 |
| TAP1 | −0.20843 | 0.95 | 0.92 | 1 |
| JUNB | −0.22587 | 0.925 | 0.918 | 1 |
| ANXA1 | 0.207417 | 0.875 | 0.818 | 1 |
| GNB2 | −0.20059 | 0.652 | 0.631 | 1 |
| GNAS | −0.22715 | 0.768 | 0.733 | 1 |
| RPS26 | −0.24149 | 1 | 0.997 | 1 |
| TOP2A | −0.21188 | 0.803 | 0.777 | 1 |
| MT-ND6 | 0.244077 | 0.944 | 0.899 | 1 |
| CENPF | −0.21818 | 0.755 | 0.714 | 1 |
| GLUL | 0.251618 | 0.809 | 0.763 | 1 |
| NEAT1 | −0.27917 | 0.89 | 0.85 | 1 |
| STAT1 | −0.20111 | 0.799 | 0.707 | 1 |
| HIST1H2AL | 0.258697 | 0.42 | 0.391 | 1 |
| HIST1H3B | −0.2463 | 0.564 | 0.539 | 1 |

TABLE 2
Genes with lower expression in cytotoxic
effector precursor CAR T cells

| | | | Proportion | |
|---|---|---|---|---|
| | | | of all other | |
| | | Proportion | CD8+ CAR T | |
| | | of | cells in the | |
| | | cytotoxic | preparation | |
| | | effector | of CAR T | |
| | Average | precursors | cells | |
| | log2 fold | expressing | expressing | Adjusted |
| Gene | change | the gene | the gene | p-value |
| CD52 | −1.24865 | 0.987 | 0.995 | 2.04E−83 |
| MAL | −1.25104 | 0.414 | 0.885 | 1.55E−76 |
| LTB | −1.30687 | 0.978 | 0.994 | 6.27E−63 |
| CAPG | −0.97276 | 0.539 | 0.928 | 1.36E−60 |
| IFITM3 | −1.08223 | 0.58 | 0.889 | 6.44E−60 |
| FLT3LG | −0.85203 | 0.696 | 0.906 | 3.8E−56 |
| CD7 | −0.69255 | 0.953 | 0.995 | 1.55E−53 |
| CD27 | −0.78335 | 0.589 | 0.869 | 4.18E−46 |
| TIMP1 | −0.82579 | 0.467 | 0.827 | 3.13E−44 |
| NOSIP | −0.69279 | 0.903 | 0.954 | 6.27E−41 |
| LIMD2 | −0.56939 | 0.991 | 0.994 | 4.04E−40 |
| IFITM1 | −0.79511 | 0.947 | 0.989 | 6.59E−40 |
| SPOCK2 | −0.69118 | 0.555 | 0.83 | 9.62E−40 |
| ITM2B | −0.57035 | 0.953 | 0.968 | 7.14E−38 |
| IFITM2 | −0.73958 | 0.884 | 0.978 | 1.33E−37 |
| EVL | −0.55847 | 0.975 | 0.992 | 8.45E−36 |
| LRRN3 | −0.64543 | 0.357 | 0.696 | 6.71E−35 |
| ITM2C | −0.67276 | 0.574 | 0.884 | 7.2E−33 |
| GPSM3 | −0.45061 | 0.972 | 0.988 | 8.11E−31 |
| SELL | −0.65363 | 0.806 | 0.939 | 2.22E−29 |
| DRAP1 | −0.38335 | 0.984 | 0.988 | 2.65E−27 |
| VSIR | −0.50018 | 0.636 | 0.802 | 4.66E−26 |
| STK17B | −0.5461 | 0.903 | 0.948 | 1.04E−25 |
| PIM1 | −0.53131 | 0.922 | 0.947 | 4.85E−25 |
| IER2 | −0.52927 | 0.978 | 0.986 | 7.36E−23 |
| PLAC8 | −0.56895 | 0.395 | 0.663 | 4.34E−22 |
| CD44 | −0.5866 | 0.746 | 0.845 | 8.84E−21 |
| LEF1 | −0.46762 | 0.335 | 0.598 | 1.16E−20 |
| RGS10 | −0.36371 | 0.959 | 0.973 | 2.86E−20 |
| CISH | −0.43359 | 0.959 | 0.975 | 1.07E−19 |
| GLIPR2 | −0.46672 | 0.411 | 0.64 | 1.18E−19 |
| BAX | −0.51351 | 0.95 | 0.966 | 1.42E−19 |
| AES | −0.50149 | 0.846 | 0.892 | 9.97E−19 |
| SUSD3 | −0.40817 | 0.611 | 0.771 | 1.55E−18 |
| XBP1 | −0.47434 | 0.84 | 0.899 | 2.9E−18 |
| PTGER2 | −0.49655 | 0.661 | 0.792 | 3.56E−18 |
| LAT | −0.37361 | 0.95 | 0.962 | 7.28E−18 |
| MCUB | −0.41125 | 0.705 | 0.801 | 9.16E−17 |
| NUCB2 | −0.43512 | 0.834 | 0.894 | 1.22E−16 |
| CDC25B | −0.46218 | 0.815 | 0.896 | 3.61E−16 |
| LCK | −0.29907 | 0.978 | 0.99 | 5.01E−15 |
| CD55 | −0.25907 | 0.737 | 0.826 | 4.8E−14 |
| SYNE2 | −0.48547 | 0.527 | 0.685 | 5.06E−14 |
| H3F3B | −0.26232 | 0.997 | 0.999 | 5.16E−14 |
| IL7R | −0.42041 | 0.254 | 0.509 | 6.09E−14 |
| CCDC107 | −0.36858 | 0.683 | 0.79 | 6.33E−14 |
| FCER1G | −0.51593 | 0.06 | 0.276 | 8.92E−14 |
| CDKN2D | −0.51625 | 0.561 | 0.693 | 1.06E−13 |
| CD69 | −0.41254 | 0.865 | 0.893 | 1.18E−13 |
| TXNIP | −0.60723 | 0.759 | 0.824 | 1.51E−13 |
| ZNF683 | −0.56535 | 0.147 | 0.38 | 3.63E−13 |
| LGALS3 | −0.55501 | 0.765 | 0.84 | 3.78E−13 |
| AQP3 | −0.54567 | 0.282 | 0.49 | 7.34E−13 |
| RIPOR2 | −0.39619 | 0.558 | 0.683 | 1.98E−12 |
| ITGB7 | −0.3665 | 0.931 | 0.941 | 2.48E−12 |
| IDH2 | −0.34829 | 0.928 | 0.949 | 3E−12 |
| PDE6G | −0.31218 | 0.135 | 0.35 | 3.13E−12 |
| PLP2 | −0.32937 | 0.969 | 0.971 | 3.77E−12 |
| CDC42SE1 | −0.37885 | 0.636 | 0.755 | 4.01E−12 |
| CD82 | −0.35963 | 0.746 | 0.848 | 5.79E−12 |
| BIN2 | −0.34041 | 0.752 | 0.839 | 7.43E−12 |
| ISG20 | −0.47474 | 0.749 | 0.839 | 8.55E−12 |
| BTG2 | −0.37565 | 0.317 | 0.503 | 8.98E−12 |
| DEF6 | −0.29931 | 0.871 | 0.893 | 1.15E−11 |
| LIMS2 | −0.34978 | 0.204 | 0.414 | 1.43E−11 |
| LEPROTL1 | −0.33673 | 0.862 | 0.874 | 2.32E−11 |
| DDAH2 | −0.36625 | 0.552 | 0.659 | 3.95E−11 |
| GIMAP7 | −0.39146 | 0.787 | 0.826 | 5.53E−11 |
| LST1 | −0.36427 | 0.351 | 0.545 | 6.47E−11 |
| ISG15 | −0.36837 | 0.636 | 0.731 | 1.1E−10 |
| CD3G | −0.30774 | 0.928 | 0.931 | 1.58E−10 |
| TRABD2A | −0.2955 | 0.298 | 0.485 | 1.82E−10 |
| METTL9 | −0.28885 | 0.455 | 0.609 | 3.5E−10 |
| DOK2 | −0.36379 | 0.734 | 0.799 | 4.73E−10 |
| TMIGD2 | −0.25116 | 0.082 | 0.267 | 7.45E−10 |
| PTPN6 | −0.34168 | 0.743 | 0.81 | 9.08E−10 |
| S100A11 | −0.31684 | 0.984 | 0.99 | 1.37E−09 |
| CD8B2 | −0.23934 | 0.085 | 0.263 | 2.17E−09 |
| R3HDM4 | −0.28743 | 0.32 | 0.49 | 2.25E−09 |
| NPDC1 | −0.33082 | 0.339 | 0.513 | 4.62E−09 |
| POLR2L | −0.33526 | 0.815 | 0.855 | 7.61E−09 |
| IL16 | −0.37384 | 0.724 | 0.782 | 8.66E−09 |
| TRBC2 | −0.45959 | 0.787 | 0.853 | 1.27E−08 |
| SARAF | −0.24257 | 0.984 | 0.986 | 1.86E−08 |
| ARF6 | −0.27542 | 0.715 | 0.775 | 1.93E−08 |
| RASA3 | −0.2566 | 0.122 | 0.296 | 2.15E−08 |
| PRKX | −0.31231 | 0.241 | 0.403 | 2.43E−08 |
| VAMP8 | −0.2757 | 0.912 | 0.926 | 2.58E−08 |
| TNFSF10 | −0.40113 | 0.812 | 0.84 | 3.61E−08 |
| CSK | −0.30944 | 0.752 | 0.798 | 4.25E−08 |
| PRELID1 | −0.30696 | 0.734 | 0.805 | 4.57E−08 |
| HAPLN3 | −0.28322 | 0.226 | 0.397 | 5.37E−08 |
| ILK | −0.27048 | 0.871 | 0.887 | 8.1E−08 |
| CYFIP2 | −0.3205 | 0.737 | 0.805 | 8.31E−08 |
| OTULINL | −0.25735 | 0.285 | 0.452 | 9.98E−08 |
| DBN1 | −0.24496 | 0.232 | 0.432 | 1.11E−07 |
| RFLNB | −0.28522 | 0.382 | 0.521 | 1.39E−07 |
| PYCARD | −0.27392 | 0.944 | 0.953 | 1.43E−07 |
| ZFP36L1 | −0.32587 | 0.524 | 0.643 | 1.52E−07 |
| DHRS3 | −0.24532 | 0.16 | 0.341 | 1.66E−07 |
| RGS19 | −0.2873 | 0.875 | 0.878 | 1.87E−07 |
| MGAT4A | −0.26911 | 0.411 | 0.542 | 1.99E−07 |
| TRAC | −0.41979 | 0.846 | 0.869 | 2.09E−07 |
| LGALS3BP | −0.285 | 0.248 | 0.427 | 2.1E−07 |
| PAXX | −0.28396 | 0.821 | 0.831 | 2.44E−07 |
| CCDC69 | −0.27172 | 0.398 | 0.545 | 4.17E−07 |
| TGFB1 | −0.29716 | 0.646 | 0.705 | 5E−07 |
| CORO1B | −0.45281 | 0.765 | 0.784 | 7E−07 |
| CYC1 | −0.26708 | 0.683 | 0.762 | 7.29E−07 |
| TESC | −0.30718 | 0.542 | 0.626 | 7.57E−07 |
| P2RX4 | −0.21569 | 0.226 | 0.391 | 8.84E−07 |
| TRBC1 | −0.55621 | 0.433 | 0.576 | 1.1E−06 |
| RGS14 | −0.28325 | 0.533 | 0.638 | 1.3E−06 |
| CXCR3 | −0.40409 | 0.674 | 0.758 | 1.53E−06 |
| HTATSF1 | −0.30866 | 0.705 | 0.754 | 1.57E−06 |
| NCR3 | −0.33899 | 0.803 | 0.862 | 1.8E−06 |
| JAK1 | −0.33277 | 0.592 | 0.655 | 2.05E−06 |
| CD248 | −0.23712 | 0.11 | 0.264 | 2.7E−06 |
| STAP1 | −0.2013 | 0.166 | 0.32 | 3.05E−06 |
| H1FX | −0.32974 | 0.238 | 0.394 | 3.52E−06 |
| ARL4C | −0.31114 | 0.893 | 0.916 | 3.77E−06 |
| UBALD2 | −0.34288 | 0.48 | 0.598 | 3.86E−06 |
| OXNAD1 | −0.28539 | 0.586 | 0.675 | 3.88E−06 |
| SERINC5 | −0.22253 | 0.166 | 0.32 | 5.39E−06 |
| RHOH | −0.27606 | 0.865 | 0.878 | 5.61E−06 |
| CCDC85B | −0.29324 | 0.915 | 0.924 | 7.66E−06 |
| PARVG | −0.27265 | 0.608 | 0.686 | 1.25E−05 |
| CD47 | −0.25549 | 0.809 | 0.834 | 1.32E−05 |
| BIN1 | −0.26725 | 0.875 | 0.876 | 1.41E−05 |
| MZT2B | −0.30041 | 0.687 | 0.741 | 1.61E−05 |
| ID3 | −0.21557 | 0.179 | 0.324 | 2.23E−05 |
| ARHGAP45 | −0.27943 | 0.574 | 0.649 | 2.66E−05 |
| C9orf78 | −0.25017 | 0.768 | 0.799 | 3E−05 |
| SATB1 | −0.33262 | 0.649 | 0.713 | 3.61E−05 |
| IRF1 | −0.39635 | 0.768 | 0.789 | 4.54E−05 |
| IL9R | −0.25383 | 0.263 | 0.412 | 5.61E−05 |
| LAIR1 | −0.22119 | 0.154 | 0.295 | 6.4E−05 |
| BHLHE40 | −0.34849 | 0.875 | 0.893 | 6.73E−05 |
| ZFP36L2 | −0.34772 | 0.451 | 0.556 | 7.94E−05 |
| LAT2 | −0.2276 | 0.313 | 0.452 | 9.53E−05 |
| RCAN3 | −0.20324 | 0.188 | 0.325 | 0.000105 |
| PNPLA2 | −0.22406 | 0.539 | 0.613 | 0.000123 |
| SAMHD1 | −0.30463 | 0.661 | 0.738 | 0.000144 |
| ARHGEF1 | −0.26377 | 0.853 | 0.865 | 0.000198 |
| CALHM2 | −0.24149 | 0.545 | 0.63 | 0.000198 |
| GPX4 | −0.20961 | 0.978 | 0.985 | 0.00021 |
| SNAI3 | −0.20847 | 0.204 | 0.339 | 0.000219 |
| IL10RA | −0.26284 | 0.596 | 0.683 | 0.000241 |
| H2AFJ | −0.2422 | 0.865 | 0.875 | 0.000247 |
| CCDC28B | −0.2345 | 0.614 | 0.661 | 0.000309 |
| PCBP1 | −0.28939 | 0.599 | 0.677 | 0.000323 |
| KLF2 | −0.25803 | 0.238 | 0.366 | 0.000369 |
| ACAP2 | −0.23747 | 0.696 | 0.735 | 0.000377 |
| ZNF580 | −0.20994 | 0.307 | 0.425 | 0.000388 |
| TXK | −0.21558 | 0.204 | 0.335 | 0.000414 |
| RBMS1 | −0.21498 | 0.414 | 0.535 | 0.000415 |
| ABLIM1 | −0.21049 | 0.188 | 0.324 | 0.000431 |
| SLFN5 | −0.2756 | 0.386 | 0.491 | 0.000605 |
| PECAM1 | −0.24255 | 0.53 | 0.615 | 0.000659 |
| IL17RA | −0.22866 | 0.295 | 0.411 | 0.000726 |
| SPINT2 | −0.2212 | 0.226 | 0.375 | 0.000731 |
| NINJ1 | −0.23699 | 0.646 | 0.714 | 0.000817 |
| ESYT1 | −0.29401 | 0.621 | 0.687 | 0.00108 |
| FAAP20 | −0.23506 | 0.555 | 0.614 | 0.001199 |
| STK4 | −0.2475 | 0.834 | 0.845 | 0.001367 |
| CD79B | −0.23133 | 0.279 | 0.394 | 0.001632 |
| AC004687.1 | −0.28343 | 0.505 | 0.592 | 0.002272 |
| PRMT2 | −0.24237 | 0.837 | 0.84 | 0.00247 |
| SMCHD1 | −0.24962 | 0.536 | 0.604 | 0.003277 |
| IKZF1 | −0.24981 | 0.621 | 0.685 | 0.003424 |
| TMEM204 | −0.20072 | 0.254 | 0.376 | 0.004011 |
| SOCS1 | −0.31725 | 0.119 | 0.236 | 0.004052 |
| MZT2A | −0.22744 | 0.749 | 0.774 | 0.004158 |
| EPSTI1 | −0.22103 | 0.235 | 0.351 | 0.004186 |
| NBDY | −0.24231 | 0.734 | 0.758 | 0.00559 |
| FOXP1 | −0.2325 | 0.367 | 0.458 | 0.007576 |
| MFNG | −0.22753 | 0.743 | 0.753 | 0.007817 |
| CD9 | −0.25404 | 0.16 | 0.278 | 0.009335 |
| CAMK2G | −0.20694 | 0.502 | 0.571 | 0.016423 |
| KLF6 | −0.24989 | 0.875 | 0.886 | 0.018043 |
| KDM5B | −0.22325 | 0.21 | 0.323 | 0.018577 |
| CDKN1B | −0.24957 | 0.498 | 0.551 | 0.023285 |
| ATP5F1D | −0.42417 | 0.345 | 0.443 | 0.023515 |
| EGLN3 | −0.21786 | 0.138 | 0.247 | 0.024964 |
| RASGRP2 | −0.20255 | 0.445 | 0.55 | 0.025196 |
| PLPP1 | −0.20464 | 0.342 | 0.435 | 0.026536 |
| LIME1 | −0.25588 | 0.643 | 0.69 | 0.02715 |
| C1orf162 | −0.24379 | 0.176 | 0.278 | 0.029955 |
| HIST1H1D | −0.34796 | 0.755 | 0.801 | 0.033642 |
| ATP6VOE2 | −0.20081 | 0.721 | 0.759 | 0.034875 |
| ZYX | −0.33507 | 0.514 | 0.57 | 0.037417 |
| ZAP70 | −0.21471 | 0.897 | 0.922 | 0.039324 |
| OSM | −0.38626 | 0.564 | 0.643 | 0.044261 |
| JAML | −0.22813 | 0.552 | 0.621 | 0.044548 |
| SELPLG | −0.2231 | 0.937 | 0.947 | 0.057103 |
| CAMK4 | −0.22306 | 0.549 | 0.606 | 0.060655 |
| PTPRCAP | −0.24292 | 0.63 | 0.643 | 0.061411 |
| LCP2 | −0.21172 | 0.875 | 0.878 | 0.064437 |
| DGKA | −0.22322 | 0.58 | 0.624 | 0.070225 |
| TRIM22 | −0.2348 | 0.304 | 0.416 | 0.076821 |
| GADD45B | −0.2582 | 0.718 | 0.721 | 0.082079 |
| SYNRG | −0.21523 | 0.552 | 0.605 | 0.089693 |
| UBE2L6 | −0.20741 | 0.887 | 0.89 | 0.095646 |
| RHEB | −0.2317 | 0.784 | 0.786 | 0.096039 |
| NFKBIA | −0.30089 | 0.423 | 0.503 | 0.107556 |
| PDLIM2 | −0.21386 | 0.373 | 0.452 | 0.122765 |
| AGTRAP | −0.26149 | 0.636 | 0.666 | 0.126042 |
| ACTN1 | −0.20834 | 0.439 | 0.535 | 0.148222 |
| MOSPD3 | −0.23456 | 0.571 | 0.604 | 0.152228 |
| BTG1 | −0.26295 | 0.715 | 0.731 | 0.15541 |
| TLN1 | −0.2203 | 0.721 | 0.729 | 0.218526 |
| NDUFA3 | −0.20752 | 0.724 | 0.747 | 0.235836 |
| SYTL1 | −0.20005 | 0.599 | 0.656 | 0.272292 |
| CCM2 | −0.21622 | 0.605 | 0.627 | 0.27608 |
| ARHGAP4 | −0.20295 | 0.687 | 0.712 | 0.333842 |
| ETS1 | −0.22926 | 0.558 | 0.601 | 0.402502 |
| PRKCB | −0.21041 | 0.577 | 0.619 | 0.512126 |
| PNRC1 | −0.2299 | 0.571 | 0.619 | 0.548926 |
| TPST2 | −0.23924 | 0.677 | 0.691 | 0.679715 |
| XIST | −0.23481 | 0.226 | 0.314 | 1 |
| COTL1 | −0.38797 | 0.803 | 0.815 | 1 |
| SUN2 | −0.23265 | 0.417 | 0.465 | 1 |
| KDELR2 | −0.22356 | 0.536 | 0.573 | 1 |
| HBEGF | −0.25179 | 0.351 | 0.424 | 1 |
| CD40LG | −0.25105 | 0.176 | 0.265 | 1 |
| MMP25- | −0.20192 | 0.489 | 0.534 | 1 |
| AS1 | | | | |
| IL2RG | −0.23299 | 0.483 | 0.525 | 1 |
| PTGER4 | −0.20866 | 0.473 | 0.505 | 1 |
| PDE4D | −0.20681 | 0.592 | 0.633 | 1 |
| MTRNR2L8 | −2.26718 | 0.279 | 0.309 | 1 |
| MT1X | 0.236902 | 0.583 | 0.596 | 1 |
| TSC22D3 | −0.22814 | 0.599 | 0.6 | 1 |
| TRAV8-2 | −0.30952 | 0.188 | 0.203 | 1 |

In certain embodiments, the pre-effector gene signature or set of differentially expressed markers comprise one or more marker genes selected from the genes listed in Table 3. In certain embodiments, the pre-effector gene signature or set of differentially expressed markers comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or all marker genes selected from the genes listed in Table 3.

TABLE 3
100 Differentially Expressed Genes between cytotoxic effector
precursor CAR T cells product effector precursors and all
other CD8+ CAR T cells in the preparation of CAR T cells
(see Tables 1 and 2 for level of expression as compared with
the other CD8+ CAR T cells in the preparation).

| | | | Proportion | |
|---|---|---|---|---|
| | | | of all other | |
| | | Proportion | CD8+ CAR T | |
| | | of | cells in the | |
| | | cytotoxic | preparation | |
| | | effector | of CAR T | |
| | Average | precursors | cells | |
| | log2 fold | expressing | expressing | Adjusted |
| Gene | change | the gene | the gene | p-value |
| PLEK | 0.364542 | 0.292 | 0.016 | 6E−292 |
| TIGIT | 0.68609 | 0.48 | 0.06 | 4.5E−208 |
| EOMES | 0.217757 | 0.226 | 0.016 | 2.2E−179 |
| ELOVL4 | 0.254744 | 0.313 | 0.031 | 1E−171 |
| MSC | 0.480344 | 0.439 | 0.067 | 2.4E−150 |
| GZMH | 0.552716 | 0.345 | 0.043 | 1.3E−148 |
| AC017002.3 | 0.524068 | 0.464 | 0.079 | 2.9E−140 |
| KLRD1 | 0.344266 | 0.27 | 0.029 | 8.1E−135 |
| CCL4 | 1.045816 | 0.295 | 0.037 | 8.5E−122 |
| LINC01943 | 1.088062 | 0.668 | 0.199 | 3.5E−118 |
| CCL3 | 0.707193 | 0.266 | 0.033 | 1.3E−111 |
| GZMK | 1.222264 | 0.48 | 0.103 | 1.6E−109 |
| HLA-DQA1 | 1.127124 | 0.624 | 0.197 | 6.77E−93 |
| HLA-DPA1 | 1.523774 | 0.934 | 0.664 | 6.08E−88 |
| HLA-DRB1 | 1.318461 | 0.987 | 0.827 | 7.48E−85 |
| CD52 | −1.24865 | 0.987 | 0.995 | 2.04E−83 |
| MAL | −1.25104 | 0.414 | 0.885 | 1.55E−76 |
| HLA-DRB5 | 1.084358 | 0.972 | 0.666 | 1.89E−76 |
| LTB | −1.30687 | 0.978 | 0.994 | 6.27E−63 |
| CD74 | 0.759303 | 1 | 0.999 | 7.78E−61 |
| CAPG | −0.97276 | 0.539 | 0.928 | 1.36E−60 |
| SH3BGRL3 | −0.73758 | 1 | 0.998 | 4.65E−60 |
| IFITM3 | −1.08223 | 0.58 | 0.889 | 6.44E−60 |
| HLA-DQA2 | 0.762851 | 0.552 | 0.188 | 1.28E−59 |
| HLA-DPB1 | 0.897488 | 0.934 | 0.658 | 3.17E−56 |
| FLT3LG | −0.85203 | 0.696 | 0.906 | 3.8E−56 |
| NPM1 | 0.607037 | 1 | 0.993 | 4.43E−55 |
| HLA-DRA | 0.901164 | 0.978 | 0.832 | 7.61E−55 |
| CD86 | 0.284958 | 0.448 | 0.136 | 3.57E−54 |
| CD7 | −0.69255 | 0.953 | 0.995 | 1.55E−53 |
| PPP2R2B | 0.25164 | 0.31 | 0.075 | 1.97E−52 |
| IFNG | 0.494949 | 0.527 | 0.193 | 5.05E−47 |
| CD27 | −0.78335 | 0.589 | 0.869 | 4.18E−46 |
| RPL12 | 0.414022 | 1 | 0.998 | 3.08E−45 |
| TIMP1 | −0.82579 | 0.467 | 0.827 | 3.13E−44 |
| FABP5 | 0.772625 | 0.934 | 0.737 | 9.2E−43 |
| HSPD1 | 0.683846 | 0.978 | 0.921 | 4.58E−42 |
| NOSIP | −0.69279 | 0.903 | 0.954 | 6.27E−41 |
| F2R | 0.38164 | 0.574 | 0.238 | 8.09E−41 |
| GSTP1 | 0.56311 | 1 | 0.961 | 1.13E−40 |
| LIMD2 | −0.56939 | 0.991 | 0.994 | 4.04E−40 |
| RPLPO | 0.345877 | 1 | 0.999 | 5.33E−40 |
| IFITM1 | −0.79511 | 0.947 | 0.989 | 6.59E−40 |
| SPOCK2 | −0.69118 | 0.555 | 0.83 | 9.62E−40 |
| HLA-DQB1 | 0.808195 | 0.824 | 0.548 | 8.36E−39 |
| ITM2B | −0.57035 | 0.953 | 0.968 | 7.14E−38 |
| TPT1 | −0.34194 | 1 | 0.999 | 1.32E−37 |
| IFITM2 | −0.73958 | 0.884 | 0.978 | 1.33E−37 |
| RPL8 | 0.283226 | 1 | 0.999 | 2.34E−36 |
| SEC11C | 0.573464 | 0.865 | 0.63 | 2.44E−36 |
| EVL | −0.55847 | 0.975 | 0.992 | 8.45E−36 |
| LRRN3 | −0.64543 | 0.357 | 0.696 | 6.71E−35 |
| SUB1 | 0.43444 | 0.997 | 0.982 | 3.27E−33 |
| ITM2C | −0.67276 | 0.574 | 0.884 | 7.2E−33 |
| CD8B | −0.57319 | 0.972 | 0.968 | 1.36E−32 |
| CST7 | 0.840627 | 0.994 | 0.944 | 2.11E−32 |
| HSP90AB1 | 0.786892 | 0.987 | 0.955 | 4.25E−32 |
| RPL5 | 0.373615 | 1 | 0.996 | 1.73E−31 |
| PARK7 | 0.366106 | 0.994 | 0.977 | 2.14E−31 |
| GPSM3 | −0.45061 | 0.972 | 0.988 | 8.11E−31 |
| SELL | −0.65363 | 0.806 | 0.939 | 2.22E−29 |
| CD3E | −0.36422 | 1 | 0.997 | 2.4E−29 |
| GALM | 0.491303 | 0.815 | 0.56 | 5.83E−29 |
| RPS27 | −0.37725 | 1 | 0.998 | 4.17E−28 |
| GAPDH | 0.463539 | 1 | 1 | 6.2E−28 |
| DRAP1 | −0.38335 | 0.984 | 0.988 | 2.65E−27 |
| RPS7 | 0.252076 | 1 | 0.998 | 4.58E−27 |
| PPIA | 0.248034 | 1 | 0.998 | 6.2E−27 |
| CENPU | 0.432574 | 0.875 | 0.651 | 2.19E−26 |
| S100A4 | −0.58301 | 0.997 | 0.996 | 2.79E−26 |
| RPL7 | 0.364734 | 1 | 0.994 | 3.85E−26 |
| VSIR | −0.50018 | 0.636 | 0.802 | 4.66E−26 |
| TMSB10 | −0.32888 | 1 | 1 | 7.17E−26 |
| STK17B | −0.5461 | 0.903 | 0.948 | 1.04E−25 |
| BCAT1 | 0.290221 | 0.574 | 0.278 | 1.18E−25 |
| EEF2 | 0.344809 | 0.997 | 0.996 | 1.24E−25 |
| UCP2 | −0.54324 | 0.966 | 0.965 | 1.91E−25 |
| B2M | −0.26349 | 1 | 1 | 2.55E−25 |
| OSTF1 | 0.423216 | 0.978 | 0.953 | 4.57E−25 |
| PIM1 | −0.53131 | 0.922 | 0.947 | 4.85E−25 |
| RNF213 | 0.534216 | 0.925 | 0.823 | 6.06E−24 |
| INSIG1 | 0.627948 | 0.893 | 0.701 | 7.57E−24 |
| OASL | 0.275431 | 0.549 | 0.272 | 1.26E−23 |
| OAZ1 | 0.305905 | 1 | 0.994 | 5.64E−23 |
| FAM3C | 0.227502 | 0.458 | 0.208 | 7.1E−23 |
| IER2 | −0.52927 | 0.978 | 0.986 | 7.36E−23 |
| PPA1 | 0.388347 | 0.984 | 0.909 | 7.75E−23 |
| CTH | 0.236701 | 0.288 | 0.104 | 1.08E−22 |
| FYN | 0.335035 | 0.467 | 0.225 | 1.98E−22 |
| RPS6 | 0.241621 | 1 | 0.999 | 3.14E−22 |
| LYAR | 0.394531 | 0.715 | 0.438 | 3.52E−22 |
| PLAC8 | −0.56895 | 0.395 | 0.663 | 4.34E−22 |
| RPL3 | 0.259626 | 1 | 0.999 | 4.38E−22 |
| RGS1 | 0.250142 | 0.295 | 0.108 | 5.48E−22 |
| EMP3 | −0.3725 | 0.997 | 0.994 | 5.92E−22 |
| TMBIM6 | 0.338155 | 0.997 | 0.981 | 6.57E−22 |
| RPL7A | 0.243024 | 1 | 0.999 | 1.29E−21 |
| FXYD5 | −0.34719 | 0.997 | 0.997 | 2.19E−21 |
| RAN | 0.339958 | 0.991 | 0.987 | 2.62E−21 |
| PRDX4 | 0.347073 | 0.759 | 0.499 | 2.79E−21 |

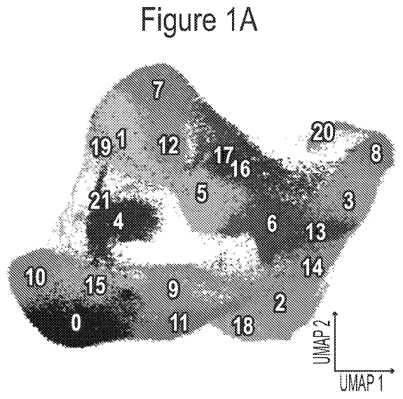

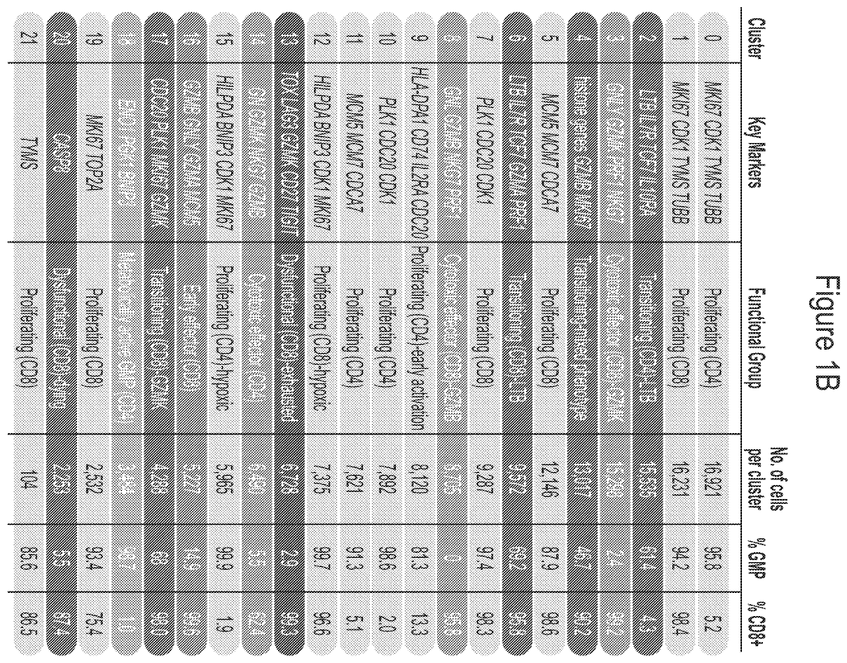

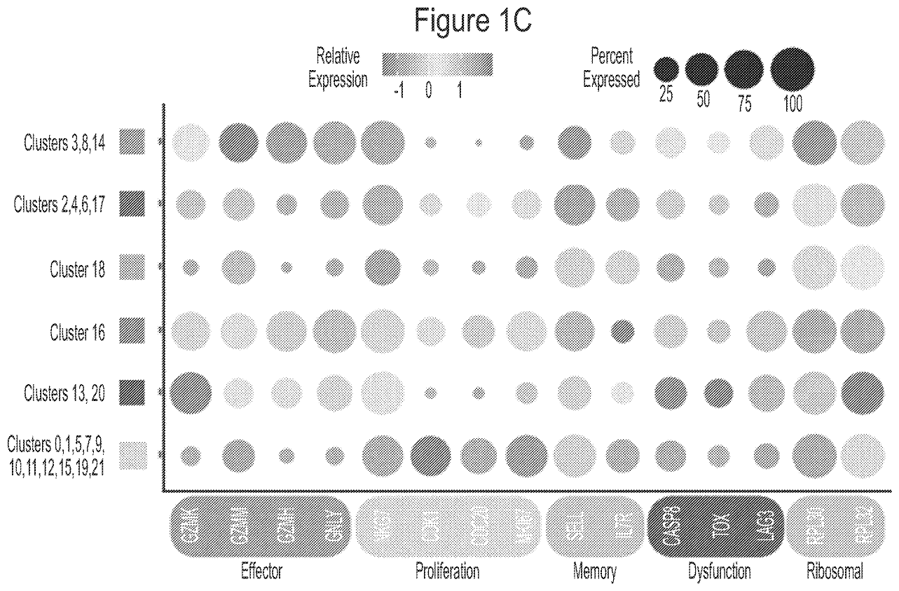

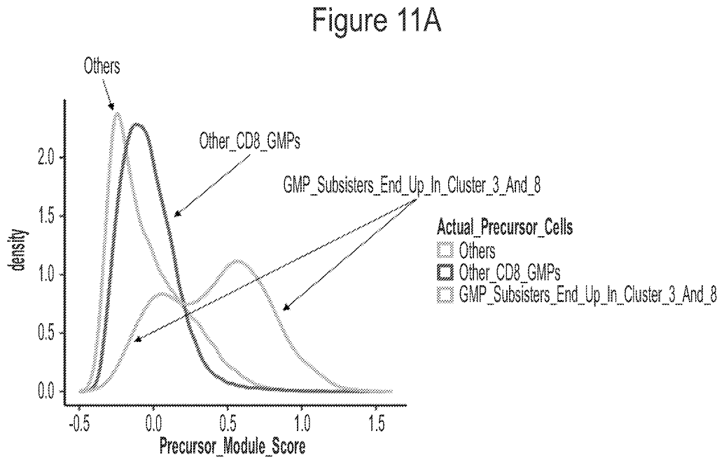

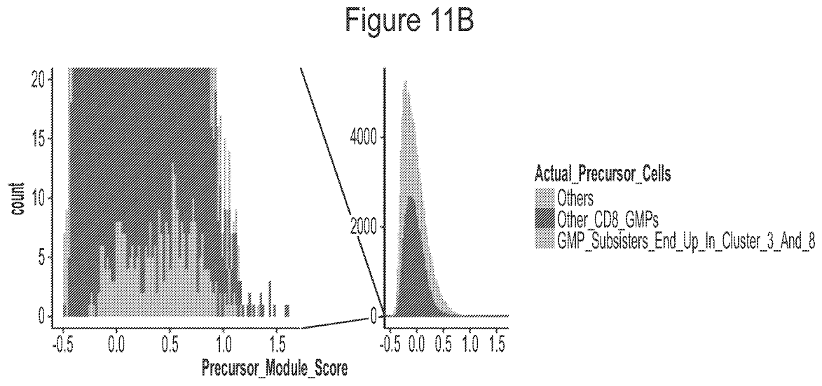

In certain embodiments, the pre-effector gene signature or set of differentially expressed markers comprise one or more marker genes selected from the genes listed in FIG. 11B. In certain embodiments, the pre-effector gene signature or set of differentially expressed markers comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or all marker genes selected from the genes listed in FIG. 1B. In certain embodiments, the pre-effector gene signature or set of differentially expressed markers comprise one or more marker genes selected from the key marker genes of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or all clusters listed in FIG. 1B. In certain embodiments, the pre-effector gene signature or set of differentially expressed markers comprise one or more marker genes selected from the key marker genes from clusters 1, 3, 5, 7, 8, 12 and/or 14 listed in FIG. 1B. In certain embodiments, the pre-effector gene signature or set of differentially expressed markers comprise one or more marker genes selected from the key marker genes from clusters 3, 8, and/or 14 listed in FIG. 1B.

In certain embodiments, the pre-effector gene signature or set of differentially expressed markers comprise one or more marker genes selected from the genes listed in Table 4. In certain embodiments, the pre-effector gene signature or set of differentially expressed markers comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or all marker genes selected from the genes listed in Table 4. In certain embodiments, the pre-effector gene signature or set of differentially expressed markers comprise one or more marker genes selected from the key marker genes of a single functional group listed in Table 4.

TABLE 4
Key marker genes associate with CAR T cell functional groups.

| Functional | |
|---|---|
| Group | Key Markers |
| Proliferating | MKI67, CDK1, TYMS, TUBB, MCM5, MCM7, CDCA7, |
| | PLK1, CDCD20, CDK1, HILPDA, BNIP3, TOP2A |
| Transitioning | LTB, GZMB, MKI67, CDC20, PLK1, GZMK |
| Functional | GNLY, GZMK, PRF1, NKG7, GZMB, KLRD1 |
| effector | |
| Dysfunctional | TOX, LAG3, CASP8, TIGIT |
| Early effector | GZMB, GNLY, GZMA, MCM5 |
| Metabolically | ENO1, PGK1, BNIP3 |
| active GMP | |

In certain embodiments, the pre-effector gene signature of differentially expressed markers comprises one or more marker genes selected from LTB, EOMES, GNLY, GZMA, GZMH, GZMK, KLRD1, IFNG, CD52, CD74, CD86, LAG3, CASP8, TOX, TIGIT, CD27, SELL, CD44, CD70, HLA-DQA1, HLA-DR, HLA-DQ, PRF1, CDC20, PLK1, MKI67, and IL-7R.

In certain embodiments, the pre-effector gene signature of differentially expressed markers comprises one or more marker genes selected from EOMES, GNLY, GZMH, GZMK, KLRD1, and IFNG.

In certain embodiments, the pre-effector gene signature of differentially expressed markers comprises one or more marker genes selected from CD52, CD74, CD86, and LAG3.

In certain embodiments, the pre-effector gene signature of differentially expressed markers comprises one or more marker genes selected from CD52, CD74, CD86, LAG3, TIGIT, SELL, and CD27.

In certain embodiments, the pre-effector gene signature of differentially expressed markers comprises one or more marker genes selected from LEF1, IL7R, SELL, and CD27.

In certain embodiments, the pre-effector gene signature of differentially expressed markers comprises one or more marker genes selected from GZMK, KLRD1, IFNG, GZMH, GNLY, and EOMES.

In certain embodiments, the pre-effector gene signature of differentially expressed markers comprises one or more marker genes selected from IFITM1, IFITM2, and IFITM3. In certain embodiments, the pre-effector gene signature of differentially expressed markers comprises one or more marker genes selected from LAG3 and/or TIGIT.

In certain embodiments, the pre-effector gene signature of differentially expressed markers comprises one or more marker genes selected from TIGIT, SELL, and CD27.

In certain embodiments, the pre-effector gene signature of differentially expressed markers comprises one or more marker genes selected from CD7, CD70, and CD86.

In certain embodiments, the pre-effector gene signature of differentially expressed markers comprises one or more marker genes selected from IFITM1, IFITM2, and IFITM3. In certain embodiments, the pre-effector gene signature comprises relatively lower expression of IFITM1, IFITM2, and IFITM3 as compared to other CAR T cells in the preparation of CAR T cells; and/or CAR T cells that that do not differentiate into a cytotoxic effector cell; and/or CAR T cells for which no evidence of lineage-specific differentiation into cytotoxic effector CAR T cells exists. This is surprising because these genes are associated with anti-viral responses.

In certain embodiments, the pre-effector gene signature of differentially expressed markers comprises one or more marker genes selected from LAG3 and TIGIT. In certain embodiments, the pre-effector gene signature comprises an increase in expression of LAG3 and/or TIGIT as compared to other CAR T cells in the preparation of CAR T cells; and/or CAR T cells that that do not differentiate into a cytotoxic effector cell; and/or CAR T cells for which no evidence of lineage-specific differentiation into cytotoxic effector CAR T cells exists. It was unexpected that there would be an increase in expression of LAG3 and/or TIGIT as they are inhibitory genes that prevent a CAR T cell from being a cytotoxic effector cell.

In certain embodiments, the pre-effector gene signature of differentially expressed markers comprises lower expression of CD27 as compared to other CAR T cells in the preparation of CAR T cells; and/or CAR T cells that do not differentiate into a cytotoxic effector cell; and/or CAR T cells for which no evidence of lineage-specific differentiation into cytotoxic effector CAR T cells exists. This is surprising because previously higher expression of CD27 was identified as a marker of strong effector CAR T cells.

In certain embodiments, the pre-effector gene signature of differentially expressed markers comprises one or more marker genes selected from TIGIT, SELL, CD27, LAG3, IFITM1, IFITM2, and IFITM3. In certain embodiments, the pre-effector gene signature of differentially expressed markers comprises one or more marker genes selected from TIGIT+, SELLlo, and CD27⁻, LAG3hi, IFITM1lo, IFITM2lo, and IFITM3lo.

In certain embodiments, the pre-effector gene signature of differentially expressed markers comprises TIGIT+, SELLlo, and CD27⁻.

In certain embodiments, the isolated CART cells are isolated from PBMCs. In certain embodiments, the isolated CAR T cells are isolated from bone marrow.

In certain embodiments, the CAR T cells are isolated once after infusion. In certain embodiments, the CAR T cells are isolated at different timepoints after infusion. In certain embodiments, the CAR T cells are isolated at multiple timepoints after infusion. In certain embodiments, the CAR T cells are isolated at about 1-25 weeks, about 1-20 weeks, about 1-18 weeks, about 1-16 weeks, about 1-12 weeks, about 1-10 weeks, about 1-8 weeks, about 1-6 weeks, about 1-5 weeks, about 1-4 weeks, about 1-3 weeks, or about 1-2 weeks post-infusion. In certain embodiments, the CAR T cells are isolated at about 1-3 weeks post-infusion. In certain embodiments, the CAR T cells are isolated at about 2-3 weeks post-infusion. In certain embodiments, the CAR T cells are isolated at 1 week post-infusion. In certain embodiments, the CAR T cells are isolated at 2 weeks post-infusion. In certain embodiments, the CAR T cells are isolated at 3 weeks post-infusion. In certain embodiments, the CAR T cells are isolated at 4 weeks post-infusion. In certain embodiments, the CAR T cells are isolated at 5 weeks post-infusion. In certain embodiments, the CAR T cells are isolated at 8 weeks post-infusion. In certain embodiments, the CAR T cells are isolated at 3 months post-infusion. In certain embodiments, the CAR T cells are isolated at 6 months post-infusion. In certain embodiments, isolating CAR T cells at multiple timepoints increases the likelihood of identifying CAR T cells that have fully differentiated into potent cytotoxic effectors. In certain embodiments, the CAR T cells are isolated during a critical window of effector differentiation and in vivo expansion, which is between 1-3 weeks post-infusion.

In one aspect, the present disclosure provides methods of determining the cytotoxic effector potential of a preparation of CAR T cells.

In certain embodiments, differentially expressed markers between the pre-effector CAR T cells of the present disclosure and the other CAR T cells in the preparation of CAR T cells; and/or CAR T cells that that do not differentiate into a cytotoxic effector cell; and/or CAR T cells for which no evidence of lineage-specific differentiation into cytotoxic effector CAR T cells exists are used to train a machine learning algorithm. In certain embodiments, the machine learning algorithm is a prediction modeling of CAR T cell effector potential in a CAR T cell product. In certain embodiments, the machine learning algorithm provides a value reflecting the probability of the analyzed CAR T cell being an effector precursor or not. In certain embodiments, the machine learning algorithm specifies whether the analyzed CAR T cell is an effector precursor or not.

In certain embodiments, the machine learning algorithm used in the methods of the present disclosure can be Support Vector Machines. In certain embodiments, the machine learning algorithm used in the methods of the present disclosure can be any classification system available in the art. In certain embodiments, the machine learning algorithm used in the methods of the present disclosure can be any model disclosed in web address: topepo.github.io/caret/available-models.html. In certain embodiments, the machine learning algorithm used in the methods of the present disclosure can be the k-nearest neighbors (KNN) algorithm. In certain embodiments, the machine learning algorithm used in the methods of the present disclosure can be the gradient boosting algorithm (GBM). In certain embodiments, the machine learning algorithm used in the methods of the present disclosure can be the SVM Linear/Radial.

In certain embodiments, the machine learning algorithm is trained using a training set of differently expressed genes comprises the top 1, 2, 3, 4, 5, 10, 15, 20, 25, 30, 35, 40, 45, 50, 55, 60, 65, 70, 75, 80, 85, 90, 95, or 100 genes (or integers in between) of the pre-effector gene signature generated by the methods of the present disclosure.

In certain embodiments, machine learning algorithm is trained using a training set of 1 to 100 differently expressed genes. In certain embodiments, machine learning algorithm is trained using a training set of 1 to 200 differently expressed genes. In certain embodiments, machine learning algorithm is trained using a training set of 1 to 300 differently expressed genes. In certain embodiments, machine learning algorithm is trained using a training set of 1 to 400 differently expressed genes. In certain embodiments, machine learning algorithm is trained using a training set of 1 to 500 differently expressed genes. In certain embodiments, machine learning algorithm is trained using a training set of 1 to 600 differently expressed genes. In certain embodiments, machine learning algorithm is trained using a training set of 1 to 700 differently expressed genes. In certain embodiments, machine learning algorithm is trained using a training set of 1 to 800 differently expressed genes. In certain embodiments, machine learning algorithm is trained using a training set of 1 to 900 differently expressed genes. In certain embodiments, machine learning algorithm is trained using a training set of 1 to 1000 differently expressed genes. In certain embodiments, machine learning algorithm is trained using a training set of 1 to 1000 differently expressed genes. In certain embodiments, machine learning algorithm is trained using a training set of 1 to 1200 differently expressed genes. In certain embodiments, machine learning algorithm is trained using a training set of 1 to 1300 differently expressed genes. In certain embodiments, machine learning algorithm is trained using a training set of 1 to 1400 differently expressed genes. In certain embodiments, machine learning algorithm is trained using a training set of 1 to 1500 differently expressed genes. In certain embodiments, machine learning algorithm is trained using a training set of 1 to 1600 differently expressed genes. In certain embodiments, machine learning algorithm is trained using a training set of 1 to 1700 differently expressed genes. In certain embodiments, machine learning algorithm is trained using a training set of 1 to 1800 differently expressed genes. In certain embodiments, machine learning algorithm is trained using a training set of 1 to 1900 differently expressed genes. In certain embodiments, machine learning algorithm is trained using a training set of 1 to 2000 differently expressed genes. In certain embodiments, machine learning algorithm is trained using a training set of 3 to 1100 differently expressed genes. In certain embodiments, machine learning algorithm is trained using a training set of 3 to 1034 differently expressed genes.

In certain embodiments, the CAR T cells having the pre-effector gene signature of the present disclosure can be identified by using flow cytometry. In certain embodiments, the CAR T cells having the pre-effector gene signature of the present disclosure can be identified by measuring protein expression of the proteins encoded by the differentially expressed genes. In certain embodiments, the protein expression can be measured by using western blotting or ELISA. In certain embodiments, the CAR T cells having the pre-effector gene signature of the present disclosure can be identified by measuring gene expression of the differentially expressed genes.

In one aspect, the present disclosure provides a method of determining the cytotoxic effector potential of a preparation of CAR T cells, the method comprising: a) acquiring single-cell gene expression data of the preparation of CAR T cells for a set of differentially expressed markers which are expressed at different levels in CAR T cells that which differentiate into cytotoxic effector cells upon administration to a subject, and b) calculating the cytotoxic effector potential indicating the likelihood of the CAR T cell preparation, when administered, to give rise to post-infusion CAR T cells and lineages thereof with cytotoxic effector function, wherein the cytotoxic effector potential of individual cells is assessed by training a machine learning algorithm to determine which cells in the preparation are effector precursors.

In another aspect, the present disclosure provides a method of determining the cytotoxic effector potential of a preparation of CAR T cells, the method comprising: a) acquiring single-cell gene expression data of the preparation of CAR T cells for a set of differentially expressed markers which are expressed at different levels in CAR T cells that differentiate into cytotoxic effector cells upon administration to a subject, and b) determining the cytotoxic effector potential of a preparation of CAR T cells or a subset of CAR T cells therefrom by (i) using flow cytometry to identify the CAR T cells of the preparation of CAR T cells that express a gene product of the set of differentially expressed markers, (ii) measuring protein expression of a gene product of the set of differentially expressed markers (e.g., via ELISA), and/or (iii) measuring gene expression of the set of differentially expressed markers.

In another aspect, the present disclosure provides a method of determining the cytotoxic effector potential of a preparation of CAR T cells, the method comprising: a) acquiring single-cell gene expression data of the preparation of CAR T cells for a set of differentially expressed markers which are expressed at different levels in CAR T cells that differentiate into cytotoxic effector cells upon administration to a subject; b) comparing the single-cell gene expression data of the preparation of CAR T cells with the pre-effector gene signature or a subset of CAR T cells therefrom with the pre-effector gene signature generated by the methods of the present disclosure; and c) determining the preparation of CAR T cells or a subset of CAR T cells therefrom has cytotoxic effector potential if the preparation of CAR T cells or a subset of CAR T cells therefrom shares at least one or more marker genes from the pre-effector gene signature. In certain embodiments, the CAR T cells which differentiate or are more likely to differentiate into cytotoxic effector cells upon administration to a subject are compared to other CAR T cells in the preparation. In certain embodiments, the CAR T cells which differentiate or are more likely to differentiate into cytotoxic effector cells upon administration to a subject are compared to the CAR T cells which do not differentiate or are less likely to differentiate into cytotoxic effector cells upon administration to a subject. In certain embodiments, the CAR T cells which differentiate or are more likely to differentiate into cytotoxic effector cells upon administration to a subject are compared to CAR T cells for which no evidence of lineage-specific differentiation into cytotoxic effector CAR T cells exists upon administration to a subject.

In another aspect, the present disclosure provides a method for enriching a preparation of CAR T cells for CAR T cells that differentiate into cytotoxic effector cells upon administration to a subject using one or more surface proteins encoded by marker genes which are expressed at different levels in CAR T cells which differentiate into cytotoxic effector cells upon administration to a subject. In another aspect, the present disclosure provides a method for enriching a preparation of CAR T cells for CAR T cells that differentiate into cytotoxic effector cells upon administration to a subject using one or more surface proteins encoded by marker genes which are expressed at different levels in CAR T cells which differentiate into cytotoxic effector cells upon administration to a subject as compared to all other CAR T cells in the preparation of CAR T cells. In another aspect, the present disclosure provides a method for enriching a preparation of CAR T cells for CAR T cells that differentiate into cytotoxic effector cells upon administration to a subject using one or more surface proteins encoded by marker genes which are expressed at different levels in CAR T cells which differentiate into cytotoxic effector cells upon administration to a subject as compared to CAR T cells which do not or less likely to differentiate into cytotoxic effector cells upon administration to a subject. In another aspect, the present disclosure provides a method for enriching a preparation of CAR T cells for CAR T cells that differentiate into cytotoxic effector cells upon administration to a subject using one or more surface proteins encoded by marker genes which are expressed at different levels in CAR T cells which differentiate into cytotoxic effector cells upon administration to a subject as compared to CAR T cells for which no evidence of lineage-specific differentiation into cytotoxic effector CAR T cells exists upon administration to a subject.

In another aspect, the present disclosure provides a method for treating a cancer in a subject in need thereof, the method comprising administering a preparation of CAR T cells determined to have cytotoxic effector potential as determined by the methods of the preset disclosure.

In another aspect, the present disclosure provides a method for treating a cancer in a subject in need thereof, the method comprising administering an enriched preparation of CAR T cells generated by the methods of the present disclosure.

### CAR T Cells

The present disclosure provides, among other things, a method for generating a pre-effector gene signature for determining the cytotoxic effector potential of a preparation of CAR T cells.

In another aspect, provided herein is an isolated CAR T cell comprising a pre-effector gene signature described above.

T cells may include, but are not limited to, thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+CD8+ T cell, or any other subset of T cells. In certain embodiments, the T cells are CD8⁺ T cells and/or CD4⁺ T cells.

In various embodiments, the T cell has been activated and/or expanded ex vivo.

In various embodiments, the T cell is an allogeneic cell. In various embodiments, the T cell is an autologous cell.

In certain embodiments, the T cell is isolated form a subject having a cancer. In some embodiments, the T cell is isolated from a subject having a tumor. In various embodiments, the cancer is a solid tumor, a brain tumor, or a leukemia. In some embodiments, the tumor can be found within, but not limited to, breast tissue, prostate tissue, bladder tissue, oral and/or dental tissue, head and/or neck tissue, stomach tissue, liver tissue, colorectal tissue, lung tissue, brain tissue, ovary, cervix, esophagus, skin, lymph nodes, and/or bone. In some embodiments, the tumor is a cancer. In some embodiments, the cancer can be, but not limited to, osteosarcoma, rhabdomyosarcoma, Ewing sarcoma and other Ewing sarcoma family of tumors, neuroblastoma, ganglioneuroblastoma, desmoplastic small round cell tumor, malignant peripheral nerve sheath tumor, synovial sarcoma, undifferentiated sarcoma, adrenocortical carcinoma, hepatoblastoma, Wilms tumor, rhabdoid tumor, high grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma and other liver cancers, head and neck cancers, leiomyosarcoma, and melanoma.

In some embodiments, the T cell is derived from a blood, marrow, tissue, or a tumor sample.

In certain aspects, the present disclosure provides a method of generating an isolated T cell described herein. The method includes genetically modifying the T cell with the polynucleotide described herein or the recombinant vector described herein. In some embodiments, the genetic modifying step is conducted via viral gene delivery. In some embodiments, the genetic modifying step is conducted via non-viral gene delivery. In some embodiments, the genetically modifying step is conducted ex vivo. In some embodiments, the method further comprises activation and/or expansion of the T cell ex vivo before, after and/or during said genetic modification.

### Chimeric Antigen Receptor (CAR)

The T cells are transduced with a CAR molecule. In certain embodiments, the pre-effector gene signature may be universal across many CAR T cell preparations. In certain embodiments, the pre-effector gene signature may be specific to a particular CAR molecule. In certain embodiments, the pre-effector gene signature is CAR generation specific (e.g., first-, second-, third-, fourth-, or fifth-generation CAR). In certain embodiments, the pre-effector gene signature may be specific to a particular to a co-stimulatory domain of a CAR molecule. In certain embodiments, the CAR molecule is an anti-CD19 CAR molecule (e.g., comprising the amino acid sequences of SEQ ID NOs: 1-7 or encoded by the nucleotide sequences of SEQ ID NOs: 8-14).

CARs are typically comprised primarily of 1) an extracellular target-binding domain, such as a single-chain variable fragment (scFv) derived from an antigen-specific monoclonal antibody, and 2) a signaling domain, such as the ζ-chain from the T cell receptor CD3. These two regions are often fused together via a transmembrane domain.

Constructs with only the antigen-specific binding region together with the lymphocyte activation domain are termed first-generation CARs. While activation of lymphocytes through a lymphocyte activation domain such as CD3ζ is sufficient to induce tumor-specific killing, such CARs fail to optimally induce T cell proliferation and survival in vivo. The second-generation CARs added co-stimulatory polypeptides to boost the CAR-induced immune response. For example, the co-stimulating polypeptide CD28 signaling domain was added to the CAR construct. This region generally contains the transmembrane region of the co-stimulatory peptide (in place of the CD3ζ transmembrane domain) with motifs for binding other molecules such as PI3K and Lck. T cells expressing CARs with only CD3ζ vs CARs with both CD3ζ and a co-stimulatory domain (e.g., CD28) demonstrated the CARs expressing both domains achieve greater activity. The most commonly used co-stimulating molecules include CD28 and 4-1BB, which promotes both T cell proliferation and cell survival. The third-generation CARs includes three signaling domains (e.g., CD3ζ, CD28, and 4-1BB), which further improves lymphocyte cell survival and efficacy. Examples of third-generation CARs include CD19 CARs, most notably for the treatment of chronic lymphocytic leukemia (Milone, M. C., et al., (2009) Mol. Ther. 17:1453-1464; Kalos, M., et al., Sci. Transl. Med. (2011) 3:95ra73; Porter, D., et al., (2011) N. Engl. J. Med. 365: 725-533, each of which is herein incorporated by reference in their entirety for all purposes). Studies in three patients showed impressive function, expanding more than a 1000-fold in vivo, and resulted in sustained remission in all three patients. Fourth-generation CARs are based on second-generation CARs, additionally modified with a constitutive or inducible expression cassette containing a transgenic protein (e.g., a cytokine). In preclinical models, the presence of a cytokine transgene greatly enhanced the efficacy of CAR-T cell therapies in comparison to second-generation CARs. Moreover, the approach was also successful at avoiding systemic toxicity—one of the most common drawbacks of CAR-T cell therapy. Fifth-generation CARs integrate an additional membrane receptor (e.g., IL-2 receptor).

In some embodiments, the CAR expressed by a CAR T cell described herein comprises an extracellular antigen-binding domain and a transmembrane domain. In some embodiments, the CAR further comprises a cytoplasmic domain. Each domain is fused in frame.

In some embodiments, the CAR expressed by a CAR T cell described herein is a first-generation CAR. In some embodiments, the CAR expressed by a CAR T cell described herein is a second-generation CAR. In some embodiments, the CAR expressed by a CAR T cell described herein is a third-generation CAR. In some embodiments, the CAR expressed by a CAR T cell described herein is a fourth-generation CAR. In some embodiments, the CAR expressed by a CAR T cell described herein is a fifth-generation CAR.

In some embodiments, the CAR is a first-generation CAR. In certain embodiments, the CAR is a second-generation CAR. In various embodiments, the CAR is a third-generation CAR. In various embodiments, the CAR is a fourth-generation CAR. In various embodiments, the CAR is a fifth-generation CAR.

### Extracellular Target-Binding Domain of the CAR

The choice of antigen-binding domain depends upon the type and number of antigens that define the surface of a target cell. For example, the antigen-binding domain may be chosen to recognize an antigen that acts as a cell surface marker on target cells associated with a particular disease state. In some embodiments, the T cells can be genetically modified to target a tumor antigen of interest by way of engineering a desired antigen-binding domain that specifically binds to an antigen (e.g., on a cancer cell). Non-limiting examples of cell surface markers that may act as targets for the antigen-binding domain in the CAR include those associated with viral, bacterial, and parasitic infections, autoimmune disease, and cancer cells.

In some embodiments, the extracellular antigen-binding domain comprises an antigen-binding polypeptide or functional variant thereof that binds to an antigen. In some embodiments, the antigen-binding polypeptide is an antibody or an antibody fragment that binds to an antigen.

In some embodiments, the antigen-binding polypeptide can be monomeric or multimeric (e.g., homodimeric or heterodimeric), or associated with multiple proteins in a non-covalent complex. In some embodiments, the extracellular target-binding domain may consist of an Ig heavy chain. In some embodiments, the Ig heavy chain can be covalently associated with Ig light chain (e.g., via the hinge and optionally the CH1 region). In some embodiments, the Ig heavy chain may become covalently associated with other Ig heavy/light chain complexes (e.g., by the presence of hinge, CH2, and/or CH3 domains). In the latter case, the heavy/light chain complex that becomes joined to the chimeric construct may constitute an antibody with a specificity distinct from the antibody specificity of the chimeric construct. In some embodiments, the entire chain may be used. In some embodiments, a truncated chain may be used, where all or a part of the CHI, CH2, or CH3 domains may be removed or all or part of the hinge region may be removed. Non-limiting examples of antigen-binding polypeptides include antibodies and antibody fragments such as e.g., murine antibodies, rabbit antibodies, human antibodies, fully humanized antibodies, single chain variable fragments (scFv), single chain antibodies, Fab fragments, F(ab′) fragments, disulfide-linked Fvs (sdFv), intrabodies, minibodies, or diabodies, camelid antibody variable domains and humanized versions, shark antibody variable domains and humanized versions, single domain antibody variable domains, nanobodies (VHHs), and camelid/camelized antibody variable domains. In some embodiments, the antigen-binding polypeptide include an scFv.

Non-limiting examples of tumor antigens that can be targeted by the CAR T cells include carbonic anhydrase EX, alpha-fetoprotein, A3, antigen specific for A33 antibody, Ba 733, BrE3-antigen, CA125, CD1, CD1a, CD3, CD5, CD15, CD16, CD19, CD20, CD21, CD22, CD23, CD25, CD30, CD33, CD38, CD45, CD74, CD79a, CD80, CD123, CD138, colon-specific antigen-p (CSAp), CEA (CEACAM5), CEACAM6, CSAp, EGFR, EGP-I, EGP-2, Ep-CAM, EphA1, EphA2, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, HER2, hypoxia inducible factor (HIF-I), Ia, IL-2, IL-6, IL-8, interleukin 13 receptor a2 (IL13Ra2), insulin growth factor-1 (IGF-I), KC4-antigen, KS-1-antigen, KS1-4, Le-Y, macrophage inhibition factor (MIF), MAGE, MUC1, MUC2, MUC3, MUC4, NCA66, NCA95, NCA90, antigen specific for PAM-4 antibody, placental growth factor, p53, prostatic acid phosphatase, PSA, PSMA, RS5, S100, TAC, TAG-72, tenascin, TRAIL receptors, Tn antigen, Thomson-Friedenreich antigens, tumor necrosis antigens, VEGF, and fibronectin-EDB (oncofetal fibronectin, FN-EDB, EDB). In certain embodiments, the CART cells of the present disclosure can bind to CD19. In certain embodiments, the CAR T cells of the present disclosure can bind to CD123.

Additional antigens that may be targeted by the CAR T cells described herein include interleukin-13 receptor subunit alpha-2 (IL-13Ra2), A kinase anchor protein 4 (AKAP-4), adrenoceptor beta 3 (ADRB3), anaplastic lymphoma kinase (ALK), immunoglobulin lambda-like polypeptide 1 (IGLL1), androgen receptor, angiopoietin-binding cell surface receptor 2 (Tie 2), B7H3 (CD276), bone marrow stromal cell antigen 2 (BST2), carbonic anhydrase IX (CAIX), CCCTC-binding factor (Zinc Finger Protein)-like (BORIS), CD171, CD179a, CD24, CD300 molecule-like family member f (CD300LF), CD38, CD44v6, CD72, CD79a, CD79b, CD97, chromosome X open reading frame 61 (CXORF61), claudin 6 (CLDN6), CS-1 (CD2 subset 1, CRACC, SLAMF7, CD319, or 19A24), C-type lectin domain family 12 member A (CLECi2A), C-type lectin-like molecule-1 (CLL-1), Cyclin B 1, Cytochrome P450 1B 1 (CYP1B 1), EGF-like module-containing mucin-like hormone receptor-like 2 (EMR2), epidermal growth factor receptor (EGFR), ERG (transmembrane protease, serine 2 (TMPRSS2) ETS fusion gene), ETS translocation-variant gene 6, located on chromosome 12p (ETV6-AML), Fc fragment of IgA receptor (FCAR), Fc receptor-like 5 (FCRL5), Fms-like tyrosine kinase 3 (FLT3), Folate receptor beta, Fos-related antigen 1, Fucosyl GM1, G protein-coupled receptor 20 (GPR20), G protein-coupled receptor class C group 5, member D (GPRC5D), ganglioside GD3, ganglioside GM3, glycoceramide (GloboH), Glypican-3 (GPC3), Hepatitis A virus cellular receptor 1 (HAVCRI), hexasaccharide portion of globoH, high molecular weight-melanoma-associated antigen (HMWMAA), human Telomerase reverse transcriptase (hTERT), interleukin 11 receptor alpha (IL-11Ra), KIT (CD117), leukocyte-associated immunoglobulin-like receptor 1 (LAIR1), leukocyte immunoglobulin-like receptor subfamily A member 2 (LILRA2), Lewis(Y) antigen, lymphocyte antigen 6 complex, locus K 9 (LY6K), lymphocyte antigen 75 (LY75), lymphocyte-specific protein tyrosine kinase (LCK), mammary gland differentiation antigen (NY-BR-1), melanoma cancer testis antigen-1 (MAD-CT-1), melanoma cancer testis antigen-2 (MAD-CT-2), melanoma inhibitor of apoptosis (ML-IAP), mucin 1, cell surface associated (MUC1), N-acetyl glucosaminyl-transferase V (NA17), neural cell adhesion molecule (NCAM), o-acetyl-GD2 ganglioside (OAcGD2), olfactory receptor 51E2 (OR51E2), p53 mutant, paired box protein Pax-3 (PAX3), paired box protein Pax-5 (PAX5), pannexin 3 (PANX3), placenta-specific 1 (PLAC1), platelet-derived growth factor receptor beta (PDGFR-beta), Polysialic acid, proacrosin binding protein sp32 (OY-TES 1), prostate stem cell antigen (PSCA), Protease Serine 21 (PRSS21), Proteasome (Prosome, Macropain) Subunit, Beta Type, 9 (LMP2), Ras Homolog Family Member C (RhoC), sarcoma translocation breakpoints, sialyl Lewis adhesion molecule (sLe), sperm protein 17 (SPA17), squamous cell carcinoma antigen recognized by T cells 3 (SART3), stage-specific embryonic antigen-4 (SSEA-4), synovial sarcoma, X breakpoint 2 (SSX2), TCR gamma alternate reading frame protein (TARP), TGS5, thyroid stimulating hormone receptor (TSHR), Tn antigen (Tn Ag), tumor endothelial marker 1 (TEM1/CD248), tumor endothelial marker 7-related (TEM7R), uroplakin 2 (UPK2), vascular endothelial growth factor receptor 2 (VEGFR2), v-myc avian myelocytomatosis viral oncogene neuroblastoma derived homolog (MYCN), Wilms tumor protein (WT1), and X Antigen Family, Member 1A (XAGE1), or a fragment or variant thereof.

In some embodiments, the extracellular antigen-binding domain further comprises a leader sequence. The leader sequence may be located at the amino-terminus of the extracellular antigen-binding domain. The leader sequence may be optionally cleaved from the antigen-binding moiety during cellular processing and localization of the CAR to the cellular membrane.

### Hinge Domain

In various embodiments, the extracellular target binding domain further comprises a hinge domain between the antigen-binding domain and the transmembrane domain. The hinge domain may be derived from all or part of naturally occurring molecules, such as from all or part of the extracellular region of CD8, CD4, or CD28, or from all or part of an antibody constant region. Alternatively, the hinge domain may be a synthetic sequence that corresponds to a naturally occurring hinge domain sequence or may be an entirely synthetic hinge domain sequence. Non-limiting examples of linker domains which may be used in accordance to the invention include a part of human CD8a, partial extracellular domain of CD28, FcγRIIIa receptor, IgG, IgM, IgA, IgD, IgE, an Ig hinge, or functional fragment thereof. The hinge may be mutated to prevent Fc receptor binding. The hinge domain can be derived from CD8a stalk, CD28, or IgG1. In certain embodiments, the hinge domain is derived from CD8a stalk. In various embodiments, the hinge domain is derived from CD28. The hinge domain can provide flexibility and accessibility between the antigen-binding moiety and the transmembrane domain.

Other hinge domains suitable for use in the present invention may be derived from an immunoglobulin IgG hinge or functional fragment, including IgG1, IgG2, IgG3, IgG4, IgM1, IgM2, IgA1, IgA2, IgD, IgE or a chimera or variant thereof.

### Leader Sequence

In various embodiments, the extracellular target-binding domain comprises a leader sequence. The leader sequence may be positioned at the N-terminus of the extracellular target-binding domain. The leader sequence may be optionally cleaved from the extracellular target-binding domain during cellular processing and localization of the CAR to the cellular membrane. Any of various leader sequences known to one of skill in the art may be used as the leader sequence. Non-limiting examples of peptides from which the leader sequence may be derived include FcεR, human immunoglobulin heavy chain variable region, CD8α, or any of various other proteins secreted by T cells. In various embodiments, the leader sequence is compatible with the secretory pathway of a T cell. In certain embodiments, the leader sequence is derived from human immunoglobulin heavy chain.

### Transmembrane Domain of the CAR

In some embodiments, the CARs expressed by the CAR T cell comprise a transmembrane domain. The transmembrane domain may be fused in frame between the extracellular target-binding domain and the cytoplasmic domain.

In some instances, the transmembrane domain can be modified by amino acid substitution, deletions, or insertions to avoid binding of proteins naturally associated with the transmembrane domain. In certain embodiments, the transmembrane domain includes additional amino acids to allow for flexibility and/or optimal distance between the domains connected to the transmembrane domain.

The transmembrane domain may be derived from a natural or from a synthetic source. Where the source is natural, the domain may be derived from any membrane-bound or transmembrane protein. Non-limiting examples of transmembrane domains of particular use in this disclosure may be derived from (i.e., comprise at least the transmembrane region(s) of) the α, β or ζ chain of the T cell receptor, CD28, CD3ε, CD3ζ, CD45, CD4, CD5, CD7, CD8, CD8α, CD9, CD16, CD22, CD33, CD37, CD40, CD64, CD80, CD86, CD134 (OX-40), CD137, or CD154. Alternatively, the transmembrane domain may be synthetic, in which case the transmembrane domain will comprise predominantly hydrophobic residues such as leucine and valine. For example, a triplet of phenylalanine, tryptophan and/or valine can be found at each end of a synthetic transmembrane domain.

In some embodiments, it will be desirable to utilize the transmembrane domain of the ζ, η or FcεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some instances, the transmembrane domain will be selected or modified by amino acid substitution to avoid-binding of such domains to the transmembrane domains of the same or different surface membrane proteins to minimize interactions with other members of the receptor complex. In other cases, it will be desirable to employ the transmembrane domain of ζ, η or FcεR1γ and -β, MIB1 (Igα.), B29 or CD3-γ, ζ, or η, in order to retain physical association with other members of the receptor complex.

### Cytoplasmic Domain of the CAR

The cytoplasmic domain can comprise one or more signaling domains. The signaling domain may be derived from CD3ζ, DAP10, DAP12, Fcε receptor Iγ chain (FCER1G), CD3δ, CD3ε, CD3γ, CD27, CD28, CD40, CD134, CD137, ICOS, MyD88, CD226, or CD79A. In certain embodiments, the signaling domain is derived from CD3ζ.

The signaling domain may activate at least one of the normal effector functions of a cell expressing the CAR.

In various embodiments, the cytoplasmic domain further comprises one or more costimulatory domains. Costimulatory domains can boost a CAR-induced immune response. Non-limiting examples of costimulatory domains include those derived from CD28, 4-1BB (CD137), CD27, CD40, CD134 (OX-40), BTLA, GITR, HVEM, CD30, CD226, CD79A, ICOS, or MyD88, or any combination thereof. In certain embodiments, the cytoplasmic domain comprises a CD28 costimulatory domain. In various embodiments, the cytoplasmic domain comprises a 4-1BB costimulatory domain.

### Accessory Genes of the CAR

In addition to the CAR construct, the CAR may further comprise an accessory gene that encodes an accessory peptide. Examples of accessory genes can include a transduced selection marker, an in vivo tracking marker, a cytokine, a suicide gene, or some other functional gene. In a specific embodiment, the CAR is co-expressed with a truncated CD19 molecule (tCD19). For example, expression of tCD19 can help determine transduction efficiency. In some embodiments, the CAR comprises the tCD19 construct. In some embodiments, the CAR does not include the tCD19 construct. In some embodiments, the tCD19 can be replaced with a functional accessory gene to enhance the effector function of the CAR containing immune effector cells. In some embodiments, the functional accessory gene can increase the safety of the CAR. In some embodiments, the CAR comprises at least one accessory gene. In some embodiments, the CAR comprises one accessory gene. In other embodiments, the CAR comprises two accessory genes. In yet another embodiment, the CAR comprises three accessory genes.

Non-limiting examples of classes of accessory genes that can be used to increase the effector function of CAR containing immune effector cells, include i) secretable cytokines (e.g., but not limited to, IL-7, IL-12, IL-15, IL-18), ii) membrane bound cytokines (e.g., but not limited to, IL-15), iii) chimeric cytokine receptors (e.g., but not limited to, IL-2/IL-7, IL-4/IL-7), iv) constitutive active cytokine receptors (e.g., but not limited to, C7R), v) dominant negative receptors (DNR; e.g., but not limited to TGFRII DNR), vi) ligands of costimulatory molecules (e.g., but not limited to, CD80, 4-1BBL), vii) antibodies, including fragments thereof and bispecific antibodies (e.g., but not limited to, bispecific T cell engagers (BiTEs)), or vii) a second CAR.

### Transduction of T Cells

The T cells can be genetically modified after stimulation/activation. In certain embodiments, the cells are modified within 12 hours, 16 hours, 24 hours, 36 hours, or 48 hours of stimulation/activation. In certain embodiments, the cells are modified within 16 to 24 hours after stimulation/activation. In certain embodiments, the cells are modified within 24 hours.

In order to genetically modify the T cell to express the CAR, the polynucleotide construct must be transferred into the T cell. Polynucleotide transfer may be via viral or non-viral gene methods. Suitable methods for polynucleotide delivery for use with the current methods include any method known by those of skill in the art, by which a polynucleotide can be introduced into an organelle, cell, tissue or organism.

In some embodiments, polynucleotides are transferred to the T cell in a non-viral vector. Non-viral vectors suitable for use in this invention include but are not limited to minicircle plasmids, transposon systems (e.g., Sleeping Beauty, piggyBac), or single or double stranded DNA molecules that are used as templates for homology directed repair (HDR) based gene editing.

Nucleic acid vaccines can be used to transfer polynucleotides into the T cells. Such vaccines include, but are not limited to non-viral polynucleotide vectors, “naked” DNA and RNA, and viral vectors. Methods of genetically modifying cells with these vaccines, and for optimizing the expression of genes included in these vaccines are known to those of skill in the art.

In certain embodiments, the T cells can be genetically modified by methods ordinarily used by one of skill in the art. In certain embodiments, the T cells can be transduced via retroviral transduction. References describing retroviral transduction of genes are Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993), each of which is incorporated herein by reference in its entirety for all purposes.

One method of genetic modification includes ex vivo modification. Various methods are available for transfecting cells and tissues removed from a subject via ex vivo modification. For example, retroviral gene transfer in vitro can be used to genetically modified cells removed from the subject and the cell transferred back into the subject. See e.g., Wilson et al., Science, 244:1344-1346, 1989 and Nabel et al., Science, 244(4910):1342-1344, 1989, both of which are incorporated herein by reference in their entity for all purposes. In certain embodiments, the T cells may be removed from the subject and transfected ex vivo using the polynucleotides (e.g., expression vectors) of the disclosure. In certain embodiments, the T cells obtained from the subject can be transfected or transduced with the polynucleotides (e.g., expression vectors) of the disclosure and then administered back to the subject.

Another method of gene transfer includes injection. In certain embodiments, a cell or a polynucleotide or viral vector may be delivered to a cell, tissue, or organism via one or more injections (e.g., a needle injection). Non-limiting methods of injection include injection of a composition (e.g., a saline based composition). Polynucleotides can also be introduced by direct microinjection. Non-limiting sites of injection include, subcutaneous, intradermal, intramuscular, intranodal (allows for direct delivery of antigen to lymphoid tissues), intravenous, intraprotatic, intratumor, intralymphatic (allows direct administration of DCs) and intraperitoneal. It is understood that proper site of injection preparation is necessary (e.g., shaving of the site of injection to observe proper needle placement).

Electroporation is another method of polynucleotide delivery. See e.g., Potter et al., (1984) Proc. Nat'l Acad. Sci. USA, 81, 7161-7165 and Tur-Kaspa et al., (1986) Mol. Cell Biol., 6, 716-718, both of which are incorporated herein in their entirety for all purposes. Electroporation involves the exposure of a suspension of cells and DNA to a high-voltage electric discharge. In certain embodiments, cell wall-degrading enzymes, such as pectin-degrading enzymes, can be employed to render the T cells more susceptible to genetic modification by electroporation than untreated cells. See e.g., U.S. Pat. No. 5,384,253, incorporated herein by reference in its entirety for all purposes.

In vivo electroporation involves a basic injection technique in which a vector is injected intradermally in a subject. Electrodes then apply electrical pulses to the intradermal site causing the cells localized there (e.g., resident dermal dendritic cells), to take up the vector. These tumor antigen-expressing dendritic cells activated by local inflammation can then migrate to lymph-nodes.

Methods of electroporation for use with this invention include, for example, Sardesai, N. Y., and Weiner, D. B., Current Opinion in Immunotherapy 23:421-9 (2011) and Ferraro, B. et al., Human Vaccines 7:120-127 (2011), both of which are hereby incorporated by reference herein in their entirety for all purposes.

Additional methods of polynucleotide transfer include liposome-mediated transfection (e.g., polynucleotide entrapped in a lipid complex suspended in an excess of aqueous solution. See e.g., Ghosh and Bachhawat, (1991) In: Liver Diseases, Targeted Diagnosis and Therapy Using Specific Receptors and Ligands. pp. 87-104). Also contemplated is a polynucleotide complexed with Lipofectamine, or Superfect); DEAE-dextran (e.g., a polynucleotide is delivered into a cell using DEAE-dextran followed by polyethylene glycol. See e.g., Gopal, T. V., Mol Cell Biol. 1985 May; 5(5):1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7(8):2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat'l Acad. Sci. USA, 84, 8463-8467); microprojectile bombardment (e.g., one or more particles may be coated with at least one polynucleotide and delivered into cells by a propelling force. See e.g., U.S. Pat. Nos. 5,550,318; 5,538,880; 5,610,042; and PCT Application WO 94/09699; Klein et al., (1987) Nature, 327, 70-73, Yang et al., (1990) Proc. Nat'l Acad Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes.

In further embodiments, T cells are genetically modified using gene editing with homology-directed repair (HDR). Homology-directed repair (HDR) is a mechanism used by cells to repair double strand DNA breaks. In HDR, a donor polynucleotide with homology to the site of the double strand DNA break is used as a template to repair the cleaved DNA sequence, resulting in the transfer of genetic information from the donor polynucleotide to the DNA. As such, new nucleic acid material may be inserted or copied into a target DNA cleavage site. Double strand DNA breaks in T cells may be induced by a site-specific nuclease. Suitable site-specific nucleases for use in the present invention include, but are not limited to, RNA-guided endonuclease (e.g., CRISPR-associated (Cas) proteins), zinc finger nuclease, a TALEN nuclease, or mega-TALEN nuclease. For example, a site-specific nuclease (e.g., a Cas9+guide RNA) capable of inducing a double strand break in a target DNA sequence is introduced to a T cell, along with a donor polynucleotide encoding a CAR of the present disclosure and optionally an additional protein (e.g., TCR or bispecific antibody).

### Isolation/Enrichment

The T cells may be autologous/autogeneic (“self”) or non-autologous (“non-self,” e.g., allogeneic, syngeneic or xenogeneic). In certain embodiments, the T cells are obtained from a mammalian subject. In other embodiments, the T cells are obtained from a primate subject. In certain embodiments, the T cells are obtained from a human subject.

T cells can be obtained from sources such as, but not limited to, peripheral blood mononuclear cells, bone marrow, lymph nodes tissue, cord blood, thymus issue, tissue from a site of infection, ascites, pleural effusion, spleen tissue, and tumors. T cells may also be generated by differentiation of stem cells. In certain embodiments, T cells can be obtained from blood collected from a subject using techniques generally known to the skilled person, such as sedimentation, e.g., FICOLL™ separation.

In certain embodiments, cells from the circulating blood of a subject are obtained by apheresis. An apheresis device typically contains lymphocytes, including T cells, monocytes, granulocytes, B cells, other nucleated white blood cells, red blood cells, and platelets. In certain embodiments, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as, but not limited to, using a semiautomated flowthrough centrifuge (e.g., Cobe 2991 cell processor, or the Baxter CytoMate). After washing, the cells may be resuspended in a variety of biocompatible buffers, cell culture medias, or other saline solution with or without buffer.

In certain embodiments, T cells can be isolated from peripheral blood mononuclear cells (PBMCs) by lysing the red blood cells and depleting the monocytes. As an example, the cells can be sorted by centrifugation through a PERCOLL™ gradient. In certain embodiments, after isolation of PBMC, both cytotoxic and helper T lymphocytes can be sorted into naive, memory, and effector T cell subpopulations either before or after activation, expansion, and/or genetic modification.

In certain embodiments, T cells can be enriched. For example, a specific subpopulation of T lymphocytes, expressing one or more markers such as, but not limited to, CD3, CD4, CD8, CD14, CD15, CD16, CD19, CD27, CD28, CD34, CD36, CD45RA, CD45RO, CD56, CD62, SELL, CD122, CD123, CD127, CD235a, CCR7, HLA-DR or a combination thereof using either positive or negative selection techniques. In certain embodiments, the T cells for use in the compositions of the disclosure do not express or do not substantially express one or more of the following markers: CD57, CD244, CD160, PD-1, CTLA4, TIM3, and LAG3.

### Stimulation/Activation

In order to reach sufficient therapeutic doses of T cell compositions, T cells are often subjected to one or more rounds of stimulation/activation. In certain embodiments, a method of producing T cells for administration to a subject comprises stimulating the T cells to become activated in the presence of one or more stimulatory signals or agents (e.g., compound, small molecule, e.g., small organic molecule, nucleic acid, polypeptide, or a fragment, isoform, variant, analog, or derivative thereof). In certain embodiments, a method of producing T cells for administration to a subject comprises stimulating the T cells to become activated and to proliferate in the presence of one or more stimulatory signals or agents.

T cells can be activated by inducing a change in their biologic state by which the cells express activation markers, produce cytokines, proliferate and/or become cytotoxic to target cells. All these changes can be produced by primary stimulatory signals. Co-stimulatory signals amplify the magnitude of the primary signals and suppress cell death following initial stimulation resulting in a more durable activation state and thus a higher cytotoxic capacity.

T cells can be activated generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In certain embodiments, the T cells can be activated by binding to an agent that activates CD3ζ.

In other embodiments, a CD2-binding agent may be used to provide a primary stimulation signal to the T cells. For example, and not by limitation, CD2 agents include, but are not limited to, CD2 ligands and anti-CD2 antibodies, e.g., the Tl 1.3 antibody in combination with the Tl 1.1 or Tl 1.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100). Other antibodies which bind to the same epitopes as any of the above described antibodies can also be used.

In certain embodiments, the T cells are activated by administering phorbol myristate acetate (PMA) and ionomycine. In certain embodiments, the T cells are activated by administering an appropriate antigen that induces activation and then expansion. In certain embodiments, PMA, ionomycin, and/or appropriate antigen are administered with CD3 induce activation and/or expansion.

In general, the activating agents used in the present disclosure includes, but is not limited to, an antibody, a fragment thereof and a proteinaceous binding molecule with antibody-like functions. Examples of (recombinant) antibody fragments are Fab fragments, Fv fragments, single-chain Fv fragments (scFv), a divalent antibody fragment such as an (Fab)2′-fragment, diabodies, triabodies (Iliades, P., et al., FEBS Lett (1997) 409, 437-441), decabodies (Stone, E., et al., Journal of Immunological Methods (2007) 318, 88-94) and other domain antibodies (Holt, L. J., et al., Trends Biotechnol. (2003), 21, 11, 484-490). The divalent antibody fragment may be an (Fab)2′-fragment, or a divalent single-chain Fv fragment while the monovalent antibody fragment may be selected from the group consisting of a Fab fragment, a Fv fragment, and a single-chain Fv fragment (scFv).

In certain embodiments, one or more binding sites of the CD3ζ agents may be a bivalent proteinaceous artificial binding molecule such as a dimeric lipocalin mutein (i.e., duocalin). In certain embodiments the receptor binding reagent may have a single second binding site, (i.e., monovalent). Examples of monovalent agents include, but are not limited to, a monovalent antibody fragment, a proteinaceous binding molecule with antibody-like binding properties or an MHC molecule. Examples of monovalent antibody fragments include, but are not limited to a Fab fragment, a Fv fragment, and a single-chain Fv fragment (scFv), including a divalent single-chain Fv fragment.

The agent that specifically binds CD3 includes, but is not limited to, an anti-CD3-antibody, a divalent antibody fragment of an anti-CD3 antibody, a monovalent antibody fragment of an anti-CD3-antibody, and a proteinaceous CD3-binding molecule with antibody-like binding properties. A proteinaceous CD3-binding molecule with antibody-like binding properties can be an aptamer, a mutein based on a polypeptide of the lipocalin family, a glubody, a protein based on the ankyrin scaffold, a protein based on the crystalline scaffold, an adnectin, and an avimer. It also can be coupled to a bead.

In certain embodiments, the activating agent (e.g., CD3-binding agents) can be present in a concentration of about 0.1 to about 10 μg/ml. In certain embodiments, the activating agent (e.g., CD3-binding agents) can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the activating agent (e.g., CD3-binding agents) is administered at a concentration of about 0.1 g/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In certain embodiments, the CD3-binding agents can be present in a concentration of 1 μg/ml.

### Expansion/Proliferation

After the T cells are activated and transduced, the cells are cultured to proliferate. T cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of T cells can include interleukins, such as IL-2, IL-7, IL-15, or IL-21 (see for example Cornish et al. 2006, Blood. 108(2):600-8, Bazdar and Sieg, 2007, Journal of Virology, 2007, 81(22):12670-12674, Battalia et al, 2013, Immunology, 139(1):109-120, each of which is incorporated herein by reference in its entirety for all purposes). Other illustrative examples for agents that may be used for the expansion of T cells are agents that bind to CD8, CD45 or CD90, such as αCD8, αCD45 or αCD90 antibodies. Illustrative examples of T cell population including antigen-specific T cells, T helper cells, cytotoxic T cells, memory T cell (an illustrative example of memory T cells are CD62L+CD8+ specific central memory T cells) or regulatory T cells (an illustrative example of Treg are CD4+CD25+CD45RA+ Treg cells).

Additional agents that can be used to expand T lymphocytes includes methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety for all purposes.

In certain embodiments, the agent(s) used for expansion (e.g., IL-2) are administered at about 20 units/ml to about 200 units/ml. In certain embodiments, the agent(s) used for expansion (e.g., IL-2) are administered at about 25 units/ml to about 190 units/ml, about 30 units/ml to about 180 units/ml, about 35 units/ml to about 170 units/ml, about 40 units/ml to about 160 units/ml, about 45 units/ml to about 150 units/ml, about 50 units/ml to about 140 units/ml, about 55 units/ml to about 130 units/ml, about 60 units/ml to about 120 units/ml, about 65 units/ml to about 110 units/ml, about 70 units/ml to about 100 units/ml, about 75 units/ml to about 95 units/ml, or about 80 units/ml to about 90 units/ml. In certain embodiments, the agent(s) used for expansion (e.g., IL-2) are administered at about 20 units/ml, about 25 units/ml, about 30 units/ml, 35 units/ml, 40 units/ml, 45 units/ml, about 50 units/ml, about 55 units/ml, about 60 units/ml, about 65 units/ml, about 70 units/ml, about 75 units/ml, about 80 units/ml, about 85 units/ml, about 90 units/ml, about 95 units/ml, about 100 units/ml, about 105 units/ml, about 110 units/ml, about 115 units/ml, about 120 units/ml, about 125 units/ml, about 130 units/ml, about 135 units/ml, about 140 units/ml, about 145 units/ml, about 150 units/ml, about 155 units/ml, about 160 units/ml, about 165 units/ml, about 170 units/ml, about 175 units/ml, about 180 units/ml, about 185 units/ml, about 190 units/ml, about 195 units/ml, or about 200 units/ml. In certain embodiments, the agent(s) used for expansion (e.g., IL-2) are administered at about 5 mg/ml to about 10 ng/ml. In certain embodiments, the agent(s) used for expansion (e.g., IL-2) are administered at about 5.5 ng/ml to about 9.5 ng/ml, about 6 ng/ml to about 9 ng/ml, about 6.5 ng/ml to about 8.5 ng/ml, or about 7 ng/ml to about 8 ng/ml. In certain embodiments, the agent(s) used for expansion (e.g., IL-2) are administered at about 5 ng/ml, 6 ng/ml, 7 ng/ml, 8 ng/ml, 9, ng/ml, or 10 ng/ml.

Conditions appropriate for T cell culture include appropriate media. Non-limiting examples of appropriate media include Minimal Essential Media (MEM), RPMI Media 1640, Lonza RPMI 1640, Advanced RPMI, Clicks, AIM-V, DMEM, a-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion.

Examples of other additives for T cell expansion include, but are not limited to, surfactant, plasmanate, pH buffers such as HEPES, and reducing agents such as N-acetyl-cysteine and 2-mercaptoethanol, Antibiotics (e.g., penicillin and streptomycin), are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In certain embodiments, expression of one or more endogenous MHC molecules in the CAR T cells is decreased or eliminated. Modified MHC molecule may be an MHC class I or class II molecule. In certain embodiments, expression of an endogenous MHC molecule may be decreased or eliminated by disrupting one or more of the MHC, 02M, TAP1, TAP2, CIITA, RFX5, RFXAP and/or RFXANK locus.

Expression of an endogenous molecules and/or any other immunogenic molecules in the T cell can be disrupted using genome editing techniques such as Clustered regularly interspaced short palindromic repeats (CRISPR)/Cas, zinc finger nucleases (ZFNs), transcription activator-like effector nucleases (TALENs), and Meganucleases. These genome editing methods may disrupt a target gene by entirely knocking out all of its output or partially knocking down its expression.

### Pharmaceutical Compositions

In another aspect, the present disclosure provides for pharmaceutical compositions comprising the CAR T cells described above. Compositions of the present disclosure include, but are not limited to, pharmaceutical compositions.

In another aspect, the present disclosure provides pharmaceutical composition comprising the CAR T described herein and a pharmaceutically acceptable carrier and/or excipient.

Excipients included in the pharmaceutical composition will have different purposes depending, for example, CAR T cells used, and the mode of administration. Examples of generally used excipients include, without limitation: saline, buffered saline, dextrose, water, glycerol, ethanol, and combinations thereof, stabilizing agents, solubilizing agents and surfactants, buffers and preservatives, tonicity agents, bulking agents, and lubricating agents. Pharmaceutical compositions comprising isolated CAR T cells will typically have been prepared and cultured in the absence of any non-human components, such as animal serum (e.g., bovine serum albumin).

Examples of pharmaceutical carriers include but are not limited to sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising CAR T cells disclosed herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising CAR T cells disclosed herein may comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the compositions are formulated to be introduced into the subject by parenteral administration, e.g., intravascular (intravenous or intraarterial), intraperitoneal, intratumoral, intraventricular, intrapleural or intramuscular administration. The parenteral preparation can be enclosed in ampoules, disposable syringes or multiple dose vials made of glass or plastic. An injectable pharmaceutical composition is preferably sterile. In some embodiments, the composition is reconstituted from a lyophilized preparation prior to administration.

In some embodiments, the CAR T cells may be mixed with substances that adhere or penetrate then prior to their administration, e.g., but not limited to, nanoparticles.

### Therapeutic Methods

In one aspect, the present disclosure provides a method for treating a disease or condition in a subject in need thereof, the method comprising administering a preparation of chimeric antigen receptor (CAR) T cells comprising a set of differentially expressed markers.

In one aspect, the present disclosure provides a method for treating a cancer in a subject in need thereof, the method comprising administering a preparation of CAR T cells determined to have cytotoxic effector potential by the methods of the present disclosure.

In certain embodiments, the present disclosure provides a method for treating a cancer in a subject in need thereof, the method comprising administering an enriched preparation of CAR T cells generated by the methods of the present disclosure. In some embodiments, the effector potential of the cells of the present disclosure is one or more of expansion, persistence, and/or tumor killing activity.

Examples of tumors include, but are not limited to, tumors of the blood and blood-forming organs (e.g., lymphoma, leukemias), and solid tumors including the soft tissue tumors (e.g., rhabdomyosarcoma), which is one that grows in an anatomical site outside the bloodstream (e.g., carcinomas). Examples of cancer include, but are not limited to, carcinoma, lymphoma, blastoma, sarcoma (e.g., Ewig sarcoma and other Ewing sarcoma family of tumors, osteosarcoma or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung), cancer of the peritoneum, hepatocellular cancer, gastric or stomach cancer (e.g., including gastrointestinal cancer, pancreatic cancer), cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, primary or metastatic melanoma, multiple myeloma and B-cell lymphoma, non-Hodgkin's lymphoma, Hodgkin's lymphoma, brain (e.g., high grade glioma, diffuse pontine glioma, ependymoma, neuroblastoma, or glioblastoma), as well as head and neck cancer, and associated metastases. Additional examples of tumors can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

In various embodiments, the tumor is selected from osteosarcoma, rhabdomyosarcoma, Ewing sarcoma and other Ewing sarcoma family of tumors, neuroblastoma, ganglioneuroblastoma, desmoplastic small round cell tumor, malignant peripheral nerve sheath tumor, synovial sarcoma, undifferentiated sarcoma, adrenocortical carcinoma, hepatoblastoma, Wilms tumor, rhabdoid tumor, high grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma and other liver cancers, head and neck cancers, leiomyosarcoma, and melanoma.

In some embodiments, the CAR T cell is an autologous cell. In some embodiments, the CAR T cell is an allogeneic cell. In cases where the CAR T is isolated from a donor, the method may further include a method to prevent graft vs host disease (GVHD) and host cell rejection.

In some embodiments, the CAR T cells may also express a CD20 polypeptide as a safety switch. Accordingly, the method may further include administering an anti-CD20 antibody to the subject for removal of the isolated cells. The anti-CD20 antibody is administered in an amount effective for sufficient removal of the isolated cells from the subject. In some embodiments, the anti-CD20 antibody is administered in an amount effective for removal of more than 50% of the isolated cells from the subject. For example, the anti-CD20 antibody may be administered in an amount effective for removal of more than 55%, more than 60%, more than 65%, more than 70%, more than 75%, more than 80%, more than 85%, more than 90%, more than 95%, more than 98%, more than 99%, or about 100% of the isolated cells from the subject. The anti-CD20 antibody may be administered in an amount effective for removal of about 50% to about 70%, about 60% to about 80%, about 70% to about 90%, or about 80% to about 100% of the isolated cells from the subject.

Non-limiting examples of anti-CD20 antibodies that can be used for removal the isolated cells include Rituximab, Ibritumomab tiuxetan, Tositumomab, Ofatumumab, Ocrelizumab, TRU-015, Veltuzumab, AME-133v, PRO131921, and Obinutuzumab. In some embodiments, the anti-CD20 antibody is Rituximab.

In some embodiments of any of the therapeutic methods described above, the composition is administered in a therapeutically effective amount. The dosages of the composition administered in the methods of the invention will vary widely, depending upon the subject's physical parameters, the frequency of administration, the manner of administration, the clearance rate, and the like. The initial dose may be larger and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve in vivo persistence of modified T cells. It is also contemplated that a variety of doses will be effective to improve in vivo effector function of modified T cells.

In some embodiments, composition comprising the CAR T cells manufactured by the methods described herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 10⁷ to 10⁹ cells/kg body weight, or 10⁷ to 10⁸ cells/kg body weight, including all integer values within those ranges. The number of modified T cells will depend on the therapeutic use for which the composition is intended for.

CAR T cells may be administered multiple times at dosages listed above.

The compositions and methods described in the present disclosure may be utilized in conjunction with other types of therapy for tumors, such as chemotherapy, surgery, radiation, gene therapy, and so forth.

It is also contemplated that when used to treat various diseases/disorders, the compositions and methods of the present disclosure can be utilized with other therapeutic methods/agents suitable for the same or similar diseases/disorders. Such other therapeutic methods/agents can be co-administered (simultaneously or sequentially) to generate additive or synergistic effects. Suitable therapeutically effective dosages for each agent may be lowered due to the additive action or synergy.

In some embodiments of any of the above therapeutic methods, the method further comprises administering to the subject one or more additional compounds selected from the group consisting of immuno-suppressives, biologicals, probiotics, prebiotics, and cytokines (e.g., GM-CSF, IFN or IL-2).

In some embodiments, the method described herein further comprises providing exogenous GM-CSF, in addition to the GM-CSF produced by the immune cells, to enhance the function of immune cells expressing a CAR or 4-1BBL of the present disclosure. Exogenous GM-CSF may be provided by, for example and not limitation, i) injection of the FDA-approved GM-CSF drug Sargramostin (Leukine™) or ii) the use of nonviral or viral vectors to express GM-CSF (e.g., FDA-approved GM-CSF expressing oncolytic virus talimogene laherparepvec [TVEC, Imlygic™]). These drugs could be given before, with, or after the administration (e.g., infusion) of the immune cells expressing a CAR or 4-1BBL of the present disclosure to patients.

As a non-limiting example, the invention can be combined with other therapies that block inflammation (e.g., via blockage of IL1, INFα/β, IL6, TNF, IL23, etc.).

The methods and compositions of the invention can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 4-1BB, OX40, etc.). The methods of the invention can be also combined with other treatments that possess the ability to modulate NKT function or stability, including but not limited to CD1d, CDid-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e). The methods of the invention can also be combined with other treatments such as midostaurin, enasidenib, or a combination thereof.

Therapeutic methods of the invention can be combined with additional immunotherapies and therapies. For example, when used for treating tumors, the compositions of the invention can be used in combination with conventional therapies, such as, e.g., surgery, radiotherapy, chemotherapy or combinations thereof, depending on type of the tumor, patient condition, other health issues, and a variety of factors. In certain aspects, other therapeutic agents useful for combination tumor therapy with the inhibitors of the invention include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In one embodiment, the modified CAR T cells of the invention can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present disclosure include, for example, aminoglutethimide, amsacrine, anastrozole, asparaginase, azacitidine, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, decitabine, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-tumor agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In various embodiments of the methods described herein, the subject is a human. The subject may be a juvenile or an adult, of any age or sex.

In accordance with the present invention there may be numerous tools and techniques within the skill of the art, such as those commonly used in molecular biology, pharmacology, and microbiology. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ.

### Non-Transitory Computer-Readable Medium

In one aspect, the present disclosure provides non-transitory computer-readable medium. In certain embodiments, the non-transitory computer-readable medium is configured to communicate with one or more processor(s) of a computational system, the non-transitory computer-readable medium including instructions thereon, that when executed by the processor(s) cause the computational system to execute certain steps related to computational processes and methods disclosed herein. Various embodiments are presented herein which include steps of a method which are explicitly included as instructions that can be executed by processor(s) and also method steps which are understood, by a person skilled in the art, as being able to be programmed as instructions that can be executed by processor(s). In one aspect, the present disclosure includes any compatible combination of such steps as understood by a person skilled in the art.

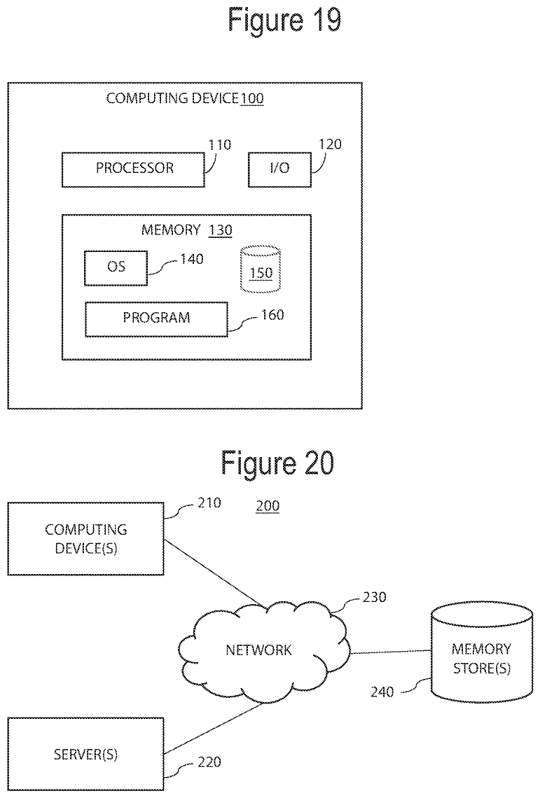

FIG. 19 illustrates a block diagram of an embodiment of a computing device 100. The computing device 100 may include one or more processor(s) 110, an input/output (I/O) device 120, a memory 130 containing an operating system (“OS”) 140, a database 150, and a program 160. The computing device 100 may be a stand-alone computing device such as a single server or may be configured as a distributed computer system including multiple servers or computers that interoperate to perform one or more of the processes and functionalities associated with the disclosed embodiments. In some embodiments, computing device 100 may further include a peripheral interface, a transceiver, a mobile network interface in communication with processor 110, a bus configured to facilitate communication between the various components of computing device 100, and a power source configured to power one or more components of computing device 100. A peripheral interface may include the hardware, firmware and/or software that enables communication with various peripheral devices, such as media drives (e.g., magnetic disk, solid state, or optical disk drives), other processing devices, or any other input source used in connection with the instant techniques. In some embodiments, a peripheral interface may include a serial port, a parallel port, a general-purpose input and output (GPIO) port, a game port, a universal serial bus (USB), a micro-USB port, a high definition multimedia (HDMI) port, a video port, an audio port, a Bluetooth™ port, an NFC port, another like communication interface, or any combination thereof.

In some embodiments, a transceiver may be configured to communicate with compatible devices and ID tags when they are within a predetermined range. A transceiver may be compatible with one or more of: RFID, NFC, Bluetooth™, low-energy Bluetooth™ (BLE), WiFi™, ZigBee™, ABC protocols or similar technologies.

A mobile network interface may provide access to a cellular network, the Internet, or another wide-area network. In some embodiments, a mobile network interface may include hardware, firmware, and/or software that allows processor 110 to communicate with other devices via wired or wireless networks, whether local or wide area, private or public, as known in the art. A power source may be configured to provide an appropriate alternating current (AC) or direct current (DC) to power components.

Processor 110 may include one or more of a microprocessor, microcontroller, digital signal processor, co-processor or the like or combinations thereof capable of executing stored instructions and operating upon stored data. Memory 130 may include, in some implementations, one or more suitable types of memory (e.g., volatile or non-volatile memory, random access memory (RAM), read only memory (ROM), programmable read-only memory (PROM), erasable programmable read-only memory (EPROM), electrically erasable programmable read-only memory (EEPROM), magnetic disks, optical disks, floppy disks, hard disks, removable cartridges, flash memory, a redundant array of independent disks (RAID), and the like) for storing files, including an operating system, application programs (including, e.g., a web browser application, a widget or gadget engine, or other applications, as necessary), executable instructions, and data. In one embodiment, the processing techniques described herein are implemented as a combination of executable instructions and data within memory 130.

Processor 110 may be one or more known processing devices, such as a microprocessor from the Pentium™ family manufactured by Intel™ or the Turion™ family manufactured by AMDm. Processor 110 may constitute a single core or multiple core processor that executes parallel processes simultaneously. For example, processor 110 may be a single core processor that is configured with virtual processing technologies. In certain embodiments, processor may use logical processors to simultaneously execute and control multiple processes. Processor 110 may implement virtual machine technologies, or other similar known technologies to provide the ability to execute, control, run, manipulate, store, etc. multiple software processes, applications, programs, etc. Other types of processor arrangements could be implemented that provide for the capabilities disclosed herein as understood by a person skilled in the pertinent art.

Computing device 100 may include one or more storage devices configured to store information used by processor (or other components) to perform certain functions related to the disclosed embodiments. In one example, computing system may include memory that includes instructions to enable processor to execute one or more applications, such as server applications, network communication processes, and any other type of application or software known to be available on computer systems. Alternatively, the instructions, application programs, etc., may be stored in an external storage or available from a memory over a network. The one or more storage devices may be a volatile or non-volatile, magnetic, semiconductor, tape, optical, removable, non-removable, or other type of storage device or tangible computer-readable medium.

In one embodiment, computing device 100 may include memory 130 that includes instructions that, when executed by processor, perform one or more processes consistent with the functionalities disclosed herein. Methods, systems, and articles of manufacture consistent with disclosed embodiments are not limited to separate programs or computers configured to perform dedicated tasks. For example, computing device 100 may include memory 130 that may include one or more programs to perform one or more functions of the disclosed embodiments. Moreover, processor 110 may execute one or more programs located remotely from computing device 100. For example, computing device 100 may access one or more remote programs, that, when executed, perform functions related to disclosed embodiments.

Memory 130 may include one or more memory devices that store data and instructions used to perform one or more features of the disclosed embodiments. Memory 130 may also include any combination of one or more databases controlled by memory controller devices (e.g., server(s), etc.) or software, such as document management systems, Microsoft™ SQL databases, SharePoint™ databases, Oracle™ databases, Sybase™ databases, or other relational databases. Memory 130 may include software components that, when executed by processor 110, perform one or more processes consistent with the disclosed embodiments. In some embodiments, memory 130 may include database 150 for storing related data to enable computing device 100 to perform one or more of the processes and functionalities associated with the disclosed embodiments.

Computing device 100 may also be communicatively connected to one or more memory devices (e.g., databases (not shown)) locally or through a network. The remote memory devices may be configured to store information and may be accessed and/or managed by computing system. By way of example, the remote memory devices may be document management systems, Microsoft™ SQL database, SharePoint™ databases, Oracle™ databases, Sybase™ databases, or other relational databases. Systems and methods consistent with disclosed embodiments, however, are not limited to separate databases or even to the use of a database.

Computing device 100 may also include one or more I/O devices 120 that may include one or more interfaces for receiving signals or input from devices and providing signals or output to one or more devices that allow data to be received and/or transmitted by computing device 100. For example, computing device 100 may include interface components, which may provide interfaces to one or more input devices, such as one or more keyboards, mouse devices, touch screens, track pads, trackballs, scroll wheels, digital cameras, microphones, sensors, and the like, that enable computing system to receive data from one or more users (such as via user device).

In example embodiments of the disclosed technology, computing device 100 may include any number of hardware and/or software applications that are executed to facilitate any of the operations. The one or more I/O interfaces may be utilized to receive or collect data and/or user instructions from a wide variety of input devices. Received data may be processed by one or more computer processors as desired in various implementations of the disclosed technology and/or stored in one or more memory devices.

While computing device 100 has been described as one form for implementing the techniques described herein, other, functionally equivalent techniques may be employed as understood by a person skilled in the pertinent art. For example, as known in the art, some or all of the functionality implemented via executable instructions may also be implemented using firmware and/or hardware devices such as application specific integrated circuits (ASICs), programmable logic arrays, state machines, etc. Furthermore, other implementations may include a greater or lesser number of components than those illustrated.

FIG. 20 illustrates a block diagram of an embodiment of a computing system 200 including computing device(s) 210, server(s) 220, memory store(s) 240, and a network 230 facilitating communication between each. The network 230 may be of any suitable type, including individual connections via the internet such as cellular or WiFi™ networks. In some embodiments, network may connect terminals, services, and mobile devices using direct connections such as radio-frequency identification (RFID), near-field communication (NFC), Bluetooth™, low-energy Bluetooth™ (BLE), WiFi™, ZigBee™, ambient backscatter communications (ABC) protocols, USB, WAN, or LAN. Because the information transmitted may be personal or confidential, security concerns may dictate one or more of these types of connections be encrypted or otherwise secured. In some embodiments, however, the information being transmitted may be less personal, and therefore the network connections may be selected for convenience over security.

Certain embodiments and implementations of the disclosed technology are described above with reference to block and flow diagrams of systems and methods and/or computer program products according to example embodiments or implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, may be repeated, or may not necessarily need to be performed at all, according to some embodiments or implementations of the disclosed technology.

These computer-executable program instructions may be loaded onto a general-purpose computer, a special-purpose computer, a processor, or other programmable data processing apparatus to produce a particular machine, such that the instructions that execute on the computer, processor, or other programmable data processing apparatus create means for implementing one or more functions specified in the flow diagram block or blocks. These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks.

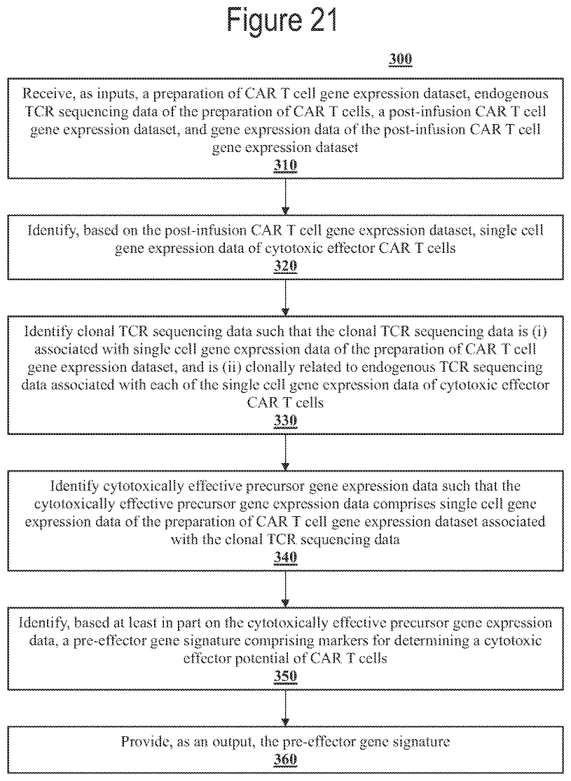

FIG. 21 is a flow diagram of a computational method 300 for providing a pre-effector gene signature.

At block 310, a preparation of CART cell gene expression dataset, endogenous TCR sequencing data of the preparation of CAR T cells, a post-infusion CAR T cell gene expression dataset, and gene expression data of the post-infusion CAR T cell gene expression dataset can be received as inputs. The preparation of CAR T cell gene expression dataset can include single cell gene expression data of CAR T cells. The endogenous TCR sequencing data of the preparation of CAR T cells can each be associated with respective single cell gene expression data of the preparation of CAR T cell single cell gene expression dataset. The post-infusion CAR T cell gene expression dataset can include single cell gene expression data of CAR T cells originating from subjects post-infusion. The preparation of CAR T cell gene expression dataset can include single cell gene expression data of CAR T cells that originate from the subjects pre-infusion and transduced with a CAR. The post-infusion CAR T cell gene expression dataset can include single cell gene expression of CAR T cells originating from the subjects at multiple times post-infusion (e.g. approximately 1, 2, 3, 4, and/or 8 weeks and/or approximately 3 months and/or 6 months post-infusion).

At block 320, single cell gene expression data of cytotoxic effector CAR T cells can be identified based on the post-infusion CAR T cell gene expression dataset (received at block 310).

At block 330, clonal TCR sequencing data can be identified such that the clonal TCR sequencing data is (i) associated with single cell gene expression data of the preparation of CAR T cell gene expression dataset (received at block 310), and is (ii) clonally related to endogenous TCR sequencing data associated with each of the single cell gene expression data of cytotoxic effector CAR T cells (identified at block 320). The cytotoxic effector potential can have a numerical value indicating likelihood of a CAR T cell, when administered, to give rise to a lineage that results in functional effector post-infusion T cells.

At block 340, cytotoxically effective precursor gene expression data can be identified such that the cytotoxically effective precursor gene expression data comprises single cell gene expression data of the preparation of CAR T cell gene expression dataset associated with the clonal TCR sequencing data (identified at block 330).

At block 350, a pre-effector gene signature comprising markers for determining a cytotoxic effector potential of CAR T cells can be identified based at least in part on the cytotoxically effective precursor gene expression data (identified at block 340).

At block 360, the pre-effector gene signature (identified at block 350) can be provided as an output. Additionally, or alternatively, the markers of the pre-effector gene signature can be provided as a training input to a machine learning algorithm, thereby resulting in a classifier configured to (i) receive, and an input, single cell gene expression data of a CAR T cell (distinct from data provided at block 310); and (ii) provide, and an output, the cytotoxic effector potential of the CAR T cell represented by the single cell gene expression data that is received as input by the classifier.

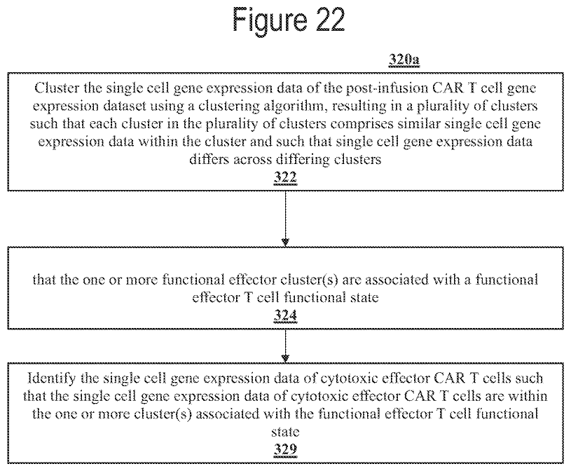

FIG. 22 is a flow diagram of a computational method 320a for identifying single cell gene expression data of cytotoxic effector CAR T cells. Block 320 illustrated in FIG. 21 can include sub-steps as illustrated in the method 320a illustrated in FIG. 22.

At block 322, the single cell gene expression data of the post-infusion CAR T cell gene expression dataset can be clustered using a clustering algorithm, resulting in a plurality of clusters such that each cluster in the plurality of clusters comprises similar single cell gene expression data within the cluster and such that single cell gene expression data differs across differing clusters. The plurality of clusters can further include single cell gene expression data of the preparation of CAR T cell gene expression dataset. The clustering algorithm can include a dimensionality reduction algorithm (e.g. uniform manifold approximation and projection (UMAP), principal component analysis (PCA), t-distributed stochastic neighbor embedding (tSNE), and/or Pseudotime analysis).

At block 324, one or more functional effector cluster(s) of the plurality of clusters can be determined such that the one or more functional effector cluster(s) are associated with a functional effector T cell functional state.

At block 326, the single cell gene expression data of cytotoxic effector CAR T cells can be identified such that the single cell gene expression data of cytotoxic effector CAR T cells are within the one or more cluster(s) associated with the functional effector T cell functional state.

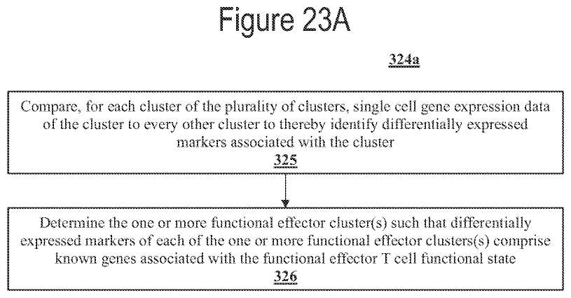

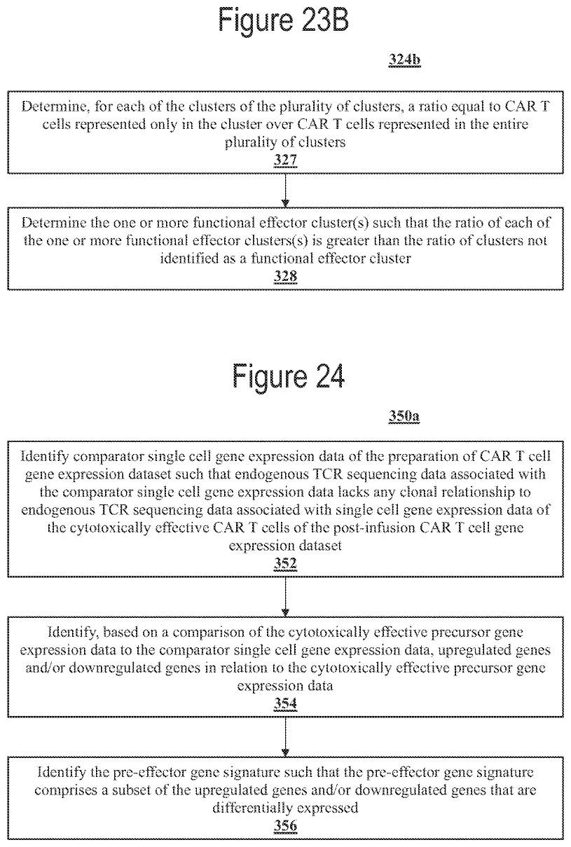

FIGS. 23A and 23B are flow diagrams of computational methods 324a, 324b for determining one or more functional effector cluster(s) associated with a functional effector T cell functional state. Block 324 illustrated in FIG. 22 can be executed using sub-steps as illustrated in the methods 324a, 324b illustrated in FIGS. 23A and 23B.

At block 325, for each cluster of the plurality of clusters, single cell gene expression data of the cluster can be compared to every other cluster to thereby identify differentially expressed markers associated with the cluster.

At block 326, the one or more functional effector cluster(s) can be determined such that differentially expressed markers of each of the one or more functional effector clusters(s) comprise known genes associated with the functional effector T cell functional state.

At block 327, for each of the clusters of the plurality of clusters, a ratio equal to CAR T cells represented only in the cluster over CAR T cells represented in the entire plurality of clusters can be determined.

At block 328, the one or more functional effector cluster(s) can be determined such that the ratio of each of the one or more functional effector clusters(s) is greater than the ratio of clusters not identified as a functional effector cluster.

FIG. 24 is a flow diagram of a computational method 350a for identifying a pre-effector gene signature. Block 350 illustrated in FIG. 21 can be executed using sub-steps as illustrated in the method 350a in FIG. 24.

At block 352, comparator single cell gene expression data of the preparation of CAR T cell gene expression dataset can be identified such that endogenous TCR sequencing data associated with the comparator single cell gene expression data lacks any clonal relationship to endogenous TCR sequencing data associated with single cell gene expression data of the cytotoxically effective CAR T cells of the post-infusion CAR T cell gene expression dataset.

At block 354, upregulated genes and/or downregulated genes, in relation to the cytotoxically effective precursor gene expression data, can be identified based on a comparison of the cytotoxically effective precursor gene expression data to the comparator single cell gene expression data. The subset of the upregulated genes and/or downregulated genes can include about 20 to about 100 genes. The subset of the upregulated genes and/or downregulated genes can include the top 1, 2, 3, 4, 5, 10, 15, 20, 25, 30, 35, 40, 45, 50, 55, 60, 65, 70, 75, 80, 85, 90, 95, or 100 genes (or integers in between) of upregulated genes and/or downregulated genes. The subset of the upregulated genes and/or downregulated genes can include the top 3 to 1,100 genes of upregulated genes and/or downregulated genes. The subset of the upregulated genes and/or downregulated genes can include the genes of Table 1, Table 2, Table 3, Table 4, FIG. 1B, genes of clusters 1, 3, 5, 7, 8, 12 and/or 14 of FIG. 1B and/or 3, 8, and/or 14 of FIG. 1B.

At block 356, the pre-effector gene signature can be identified such that the pre-effector gene signature comprises a subset of the upregulated genes and/or downregulated genes that are differentially expressed.

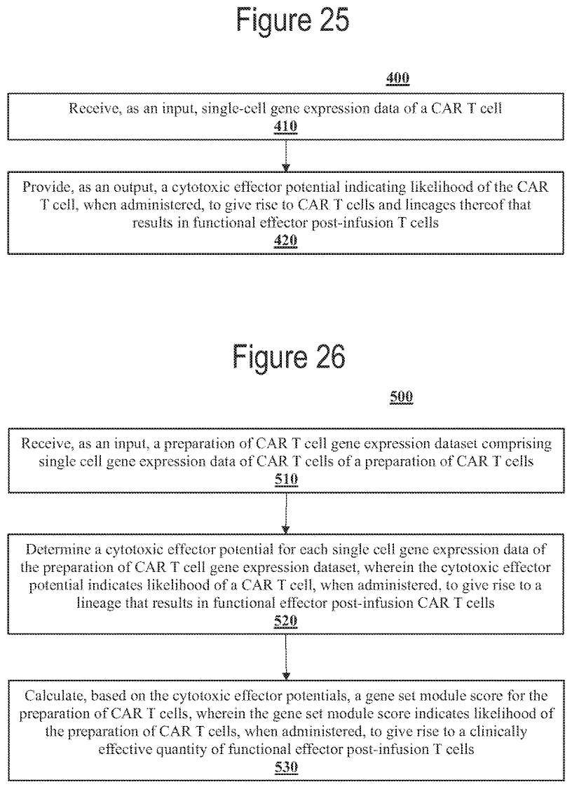

FIG. 25 is a flow diagram of a computational method 400 for providing a cytotoxic effector potential.

At block 410, single-cell gene expression data of a CAR T cell can be received as an input.

At block 420, a cytotoxic effector potential can be provided as an output. The cytotoxic effector potential can indicate likelihood of the CAR T cell, when administered, to give rise to CAR T cells and lineages thereof that results in functional effector post-infusion T cells. The instructions may, but need not require endogenous T cell receptor (TCR) sequencing data associated with the single-cell gene expression data in order to provide the cytotoxic effector potential. The CAR T cell (provided as input at block 410) may be identified as a cytotoxically effective precursor.

The method 400 may, but need not, be based on a machine learning algorithm provided training set of markers of the pre-effector gene signature as a training input. The training set of markers can include the genes of Table 1, Table 2, Table 3, Table 4, FIG. 1B, genes of clusters 1, 3, 5, 7, 8, 12 and/or 14 of FIG. 1B and/or 3, 8, and/or 14 of FIG. 1B. The training set of markers can include the top 1, 2, 3, 4, 5, 10, 15, 20, 25, 30, 35, 40, 45, 50, 55, 60, 65, 70, 75, 80, 85, 90, 95, or 100 genes (or integers in between) of the pre-effector gene signature. The training set of markers can include the top 3 to 1,100 genes of the pre-effector gene signature.

FIG. 26 is a flow diagram of a computational method 500 for calculating gene set module score.

At block 510, a preparation of CAR T cell gene expression dataset can be received as an input. The preparation of CAR T cell gene expression dataset can comprise single cell gene expression data of CAR T cells of a preparation of CAR T cells.

At block 520, a cytotoxic effector potential for each single cell gene expression data of the preparation of CAR T cell gene expression dataset (received at block 510) can be determined. The cytotoxic effector potential can indicate likelihood of a CAR T cell, when administered, to give rise to a lineage that results in functional effector post-infusion CAR T cells.

At block 530, a gene set module score for the preparation of CAR T cells can be calculated based on the cytotoxic effector potentials (determined at block 520). The gene set module score can indicate likelihood of the preparation of CAR T cells, when administered, to give rise to a clinically effective quantity of functional effector post-infusion T cells.

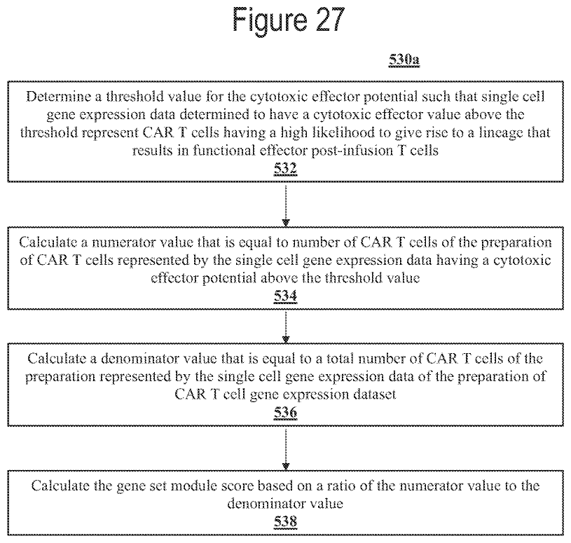

FIG. 27 is a flow diagram of a computational method 530a for calculating gene set module score. Block 530 illustrated in FIG. 26 can be executed using sub-steps as illustrated in the method 530a in FIG. 27.

At block 532, a threshold value for the cytotoxic effector potential can be determined such that single cell gene expression data determined to have a cytotoxic effector value above the threshold represent CAR T cells having a high likelihood to give rise to a lineage that results in functional effector post-infusion T cells.

At block 534, a numerator value can be calculated that is equal to number of CAR T cells of the preparation of CAR T cells represented by the single cell gene expression data having a cytotoxic effector potential above the threshold value.

At block 536, a denominator value can be calculated that is equal to a total number of CAR T cells of the preparation represented by the single cell gene expression data of the preparation of CAR T cell gene expression dataset.

At block 538, the gene set module score can be calculated based on a ratio of the numerator value to the denominator value.

As an example, embodiments or implementations of the disclosed technology may provide for a computer program product, including a computer-usable medium having a computer-readable program code or program instructions embodied therein, said computer-readable program code adapted to be executed to implement one or more functions specified in the flow diagram block or blocks. Likewise, the computer program instructions may be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special-purpose hardware and computer instructions.

Certain implementations of the disclosed technology are described above with reference to customer devices that may include mobile computing devices. Those skilled in the art will recognize that there are several categories of mobile devices, generally known as portable computing devices that can run on batteries but are not usually classified as laptops. For example, mobile devices can include, but are not limited to portable computers, tablet PCs, internet tablets, PDAs, ultra-mobile PCs (UMPCs), wearable devices, and smart phones. Additionally, implementations of the disclosed technology can be utilized with internet of things (IoT) devices, smart televisions and media devices, appliances, automobiles, toys, and voice command devices, along with peripherals that interface with these devices.

### Kits

For practicing the methods of the present invention, the compositions as described herein may optionally be provided to a user as a kit. For example, a kit of the invention contains one or more reagents, packaging material, containers for holding the components of the kit, and an instruction set or user manual detailing preferred methods of using the kit components.

In one aspect, the invention relates to a kit for determining the cytotoxic effector potential of a preparation of CAR T cells comprising a set or reagents that detects the level of one or more genes listed in Table 1, 2, 3, and/or 4 and/or FIG. 1B or the genes identified in any one of the embodiments of the present disclosure and/or detect the level of expression of polypeptides encoded by the genes and optionally instructions for using said kit.

In certain embodiments, the set of reagent detects the expression of mRNA expressed from said set of genes. In certain embodiments, the set of reagents detects the expression of polypeptides encoded by said set of genes.

In certain embodiments, the kit may contain compositions comprising the CAR T cells described herein. In certain embodiments, the composition may be a pharmaceutical composition. In certain embodiments, the present disclosure provides kits comprising a pharmaceutical composition comprising the CAR T described herein and a pharmaceutically acceptable carrier and/or excipient.

### EXAMPLES

The following examples are provided to further describe some of the embodiments disclosed herein. The examples are intended to illustrate, not to limit, the disclosed embodiments.

### Materials and Methods

### Study Design and Participants

The samples were obtained from subjects enrolled on a single institution Phase I/II clinical study evaluating the safety and efficacy of escalating doses of autologous CD19-CAR T cells in pediatric/adolescent & young adult subjects ≤21 years old with relapsed/refractory CD19-positive B-ALL (SJCAR19; NCT03573700). The protocol was approved by the St. Jude Children's Research Hospital institutional review board (IRB). Written informed consent/assent were obtained from all participants/parents in accordance with institutional guidelines and the Declaration of Helsinki. The clinical grade lentiviral vector encoding the CD19.4-1BBz CAR, and CD19-CAR T cell products were manufactured at the Children's Good Manufacturing Practice facility of St. Jude. The study, lentiviral vector, and the manufacturing of autologous CD19-CAR T cells is described in detail elsewhere (Talleur A C, et al., Preferential expansion of CD8+CD19-CAR T cells post infusion and role of disease burden on outcome in pediatric B-ALL).

Protocol treatment included lymphodepletion (fludarabine [25 mg/m², days−4 to −2] and cyclophosphamide [900 mg/m², day−2]) followed by CAR T cell infusion (day 0). The first six patients received 1×10⁶ CAR-positive T cells/kg, and starting with patient 7, 3×10⁶ CAR-positive T cells/kg were infused. The highest dose provided only three-fold more cells than the lowest dose, and there were no major differences observed between dosages (Blood Adv. 2022; bloodadvances.2021006293). Response, at 4 weeks post infusion, was categorized as complete response (CR), either minimal-residual disease (MRD)-negative or MRD-positive, or no response (NR). When available for a given patient, MRD-testing included flow-cytometry, RT-PCR and/or next-generation-sequencing (NGS; Adaptive Biotechnologies) techniques.

Peripheral blood samples analyzed for this study were collected at weeks 1-4, 8, and months 3 and 6 post infusion; bone marrow aspirates were obtained at week 4 and month 3 post infusion. qPCR assays for the CD19-CAR transgene were performed as previously described (Talleur A C, et al., Preferential expansion of CD8+CD19-CAR T cells post infusion and role of disease burden on outcome in pediatric B-ALL).

### Sample Processing and T Cell Sorting

Blood samples were drawn into CPT tubes and were sent to be processed by the Immune Monitoring Core within BMTCT. PBMCs were separated from whole blood via centrifugation, and remaining red blood cells (RBCs) were lysed for 1 minute at room temperature. Bone marrow cells were collected, and RBCs were lysed for 5 minutes at room temperature. Cryopreserved pre-infusion CART cells were thawed at 37° C. and washed prior to staining. Cells were blocked with Human TruStain FcX (Biolegend; Cat #422302) and then stained with a human CD19 CARDetection reagent (Miltenyi; Cat #130-115-965) for 10 minutes at room temperature. Cells were then washed twice and incubated with an anti-biotin antibody to label the CARdetection reagent (Miltenyi; Cat #130-111-068) and a cocktail of surface antibodies targeting CD45, CD3, CD14, CD16, CD8, and CD4 for 10 minutes at room temperature. Cells were washed twice, stained with DAPI, and then resuspended in fluorescence activated cell sorting (FACS) buffer for cell sorting using the FACS Aria III (BD Biosciences). Total CD3+ T cells were sorted, except when the CAR:CAR- or CD4:CD8 ratios were highly skewed. In this case, populations were sorted separately to be combined at equal cell ratios.

The antibodies used were CD45-FITC (BD Biosciences; Cat #555482), CD3-APC (Tonbo Biosciences; Cat #20-0038-1500), CD14-APC-Cy7 (BD Biosciences; Cat #333945), CD16-APC-Cy7 (BD Biosciences; Cat #557758), CD8-BV510 (BD Biosciences; Cat #563919), CD4-BV786 (Biolegend; Cat #317442).

### Single-Cell Gene Expression and V(D)J Sequencing

Sorted cells from each sample were counted and assayed for viability via hemocytometer. Because GMP samples were always obtained after cryopreservation, sorted CD4+ and CD8+ populations were sometimes differentially pooled in order to obtain specific CD4:CD8 ratios; however, post-infusion samples were always analyzed fresh, and the CD4:CD8 ratios remained unmanipulated in order to maintain an accurate representation of the CD4+ and CD8+ CAR T cell response dynamics within patients. Sorted cells were processed using the 10× Genomics Chromium controller and the Chromium Single Cell V(D)J 5′ reagents kits (vi) (10× Genomics; Part #1000014/1000006). T cell receptor V(D)J cDNA was enriched using the Chromium Single Cell V(D)J Enrichment kit for human T cells (10× Genomics; Part #1000005). V(D)J libraries and 5′ Gene Expression libraries were generated using 10× Genomics library preparation kits. Quantification and quality assessment were completed using the Agilent Tapestation and Agilent High Sensitivity DNA reagents (Agilent; Part #5067-5593) and Screen Tapes (Agilent; Part #5067-5592). Libraries were sequenced on the Illumina NovaSeq platform (gene expression sequencing configuration: 26-8-0-91; TCR sequencing configuration: 150-8-0-150).

### Single Cell Gene Expression and V(D)J Analysis

Single-cell gene expression data were processed using CellRanger (v.3.1.0, 10× Genomics) with the corresponding GRCh38 reference (v.3.0.0) modified to include the first 825 nucleotide bases of the CD19-CAR transcript. Resulting gene expression matrices were aggregated, again using CellRanger with default parameters (i.e., with depth normalized by the number of mapped reads), resulting in an average of 46,143 reads per cell, over 95% of reads within cells, 1,886 median genes per cell, and 5,955 median UMI counts per cell. Single-cell TCR data were processed using the same version of CellRanger with the corresponding GRCh38 V(D)J reference (v. 3.1.0). Across all V(D)J reactions, the median number of mean TCR read pairs per cell was 11,179 (minimum: 4,419). All included gene expression and V(D)J reactions passed CellRanger quality control metrics.

Aggregated gene expression data were subsequently analyzed using Seurat (Hao Y. et al., Cell. 2021; 184:3573-3587.e29) within the R statistical environment, including cells with a minimum of 300 detected genes and genes found in a minimum of three cells. Standard filtering and processing procedures were utilized prior to downstream analyses. Specifically, we excluded potential doublets and dying cells by filtering out cells with >=5,000 genes and >=10% mitochondrial content, respectively. Data were log-normalized using default parameters. Any cell that did not contain at least one CD19-CAR UMI was then excluded in order to exclusively analyze transcriptionally defined CD19-CAR T cells. CD19-CAR T cells utilized for downstream analyses exhibited 11,259 median UMI counts per cell, 2,992 median genes per cell, and a median percent of expression owed to mitochondrial genes of 4%.

The top 2,000 variable features were identified using the vst method after excluding TCR and IG genes, and data were subsequently scaled using default parameters. In order to correct for potential batch effects accruing over the 1.5-year course of data acquisition from fresh samples, fastMNN algorithm (Haghverdi L. et al., Nat Biotechnol. 2018; 36:421-7) was used, as implemented in Seurat, across each 10× reaction with default parameters. The resulting mnn reduction dimensions were subsequently utilized to identify transcriptional clusters via Seurat's implemented shared nearest-neighbors approach and to generate Uniform Manifold Approximation and Projection (UMAP), again using default parameters. To infer whether individual cells were CD4+, CD8+, or of a potential mixed phenotype, it was considered whether CD4 or CD8 expression was greater in the cell and also compared to automated annotations from the NovershternHematopoieticData reference (Novershtern N. et al., Cell. 2011; 144:296-309) using SingleR (“consensus annotation”) (Aran D. et al., Nat Immunol. Nature Publishing Group; 2019; 20:163-72). For estimating the relative abundance of CD4+ and CD8+ cells in clusters, SignacX (Chamberlain M. et al., bioRxiv; 2021) was used. However, due to the limitations of these cell-specific annotation methods, for downstream analyses focused on either CD4+ or CD8+ cells, it was assumed all cells in a primarily CD8+ cluster were CD8+ and all cells in a primarily CD4+ cluster were CD4+, with cells in a mixed phenotype cluster excluded from either.

To identify lineages, the TCR sequencing information was leveraged associated with the present single-cell gene expression data. Briefly, the filtered contig annotations provided by CellRanger were integrated with the processed gene expression objects by linking TCR cell barcodes to gene expression cell barcodes. For each patient, defined potential clonal lineages were then identified using four distinct approaches. The first two approaches were a only, classifying two CAR cells as lineages when all a observed alleles match exactly while disregarding the β chain, and β only, classifying two CAR cells as lineages based on matching in only the β chain. Neither of these approaches fully represents the TCR by neglecting the contribution of the other chain. Since cells can differentially express distinct alleles of both the α and ρ chains of the TCR, the strictest definition of a lineage would be cases where all alphas and all betas must match between two or more cells. The fewest number of lineages were detected when requiring exact matches of all alleles, and thus defined lineages using a “one-from-each” approach (with one α & one β), where cells that match their most highly expressed (as a stringency filter) α and β chains are designated as lineages.

Pseudotime analysis was conducted using Monocle2 (version 2.20.0) which utilizes DDRTree (Discriminative Dimensionality Reduction via learning a Tree) for dimensionality reduction (Qiu X et al., Nat Methods. 2017; 14:309-15). DDRTree is a reversed graph embedding technique to reduce the data's dimensionality and make the single cell data into a tree format, so that the trajectories can be visualized and the pseudome calculated. Downsampling was necessary for this analysis due to computational limitations. 368 cells were randomly selected (the number of all the cells at the month 3 time point) from each time point, and all 840 cells with TCRs matching known pre- to post-infusion lineages were additionally added regardless of cluster designation. Pseudotime states were generated based on internal clustering by the pseudotime analysis.

### Effector CAR Product Precursor Signature Validation

Cryopreserved CAR product cells were thawed at 37° C. and washed. Cells were, then, stained with the CD19 CARDetection reagent (Miltenyi; Cat #130-115-965) for 10 minutes at room temperature followed by staining with an anti-biotin antibody conjugated to APC (Miltenyi; Cat #130-110-952) and a cocktail of antibodies (CD3, CD8, CD27, CD62L, CD25, and TIGIT) and a viability dye (Tonbo Biosciences; Cat #13-0870-T100). After staining for 10 minutes at room temperature, the cells were washed twice and resuspended in FACS buffer for sorting on the FACS Aria Fusion (BD Biosciences). CD8+ CAR T cells with either the predicted effector surface profile (TIGIT+, CD62Llo and CD27neg) or the opposite, non-effector precursor profile (TIGIT−, CD62L+ and CD27+) were sorted into complete RPMI media. Cells were lysed with Trizol and for bulk TCR repertoire sequencing.

Antibodies used were anti-human CD3-APC-H7 (BD Pharmingen; Cat #560176), anti-human CD8-BV785 (Biolegend; Cat #344740), anti-human CD27-PE-CF594 (BD Horizon; Cat #562297), anti-human CD62L-BV421 (Biolegend; Cat #304828), anti-human CD25-VioBright FITC (Miltenyi Biotec; Cat #130-113-283), and anti-human TIGIT-PE (Biolegend; Cat #372703).

### TCR Repertoire Sequencing

Bulk TCRalpha and TCRbeta repertoire sequencing was performed using the 5′RACE protocol adapted from (Egorov et al 2015, website jimmunol.org/content/194/12/6155). In brief, total RNA was isolated from sorted cells with Trizol reagent (Invitrogen; Cat #15596026) using the manufacturer's protocol. cDNA synthesis was performed with SmartScribe kit (TakaraBio; Cat #639537) with TCRalpha and TCRbeta C-segment specific primers and template switching oligonucleotide with UMI sequence. cDNA synthesis product was purified using Ampure XP kit (Beckman Coulter; Cat #A63880), and amplified in two rounds of PCR with Q5 HotStart high-fidelity polymerase kit (NEB; Cat #M0493S). Adapters for illumina sequencing were ligated with KAPA HyperPrep kit (Roche; Cat #07962363001). The libraries were sequenced on illumina Novaseq platform (2×150 paired end sequencing).

Sample demultiplexing and UMI-guided assembly were conducted using MiGEC (v.1.2.9) CheckoutBatch (with-ute flags) and AssembleBatch (with-force-overseq set to 1) functions, respectively (Shugay M. et al., Nat Methods. 2014; 11:653-5). Individual sample assemblies were further parsed and annotated using the MiXCR (v.3.0.13) analyze amplicon function (-species hs-starting-material rna-5-end no-v-primers-3-end c-primers-adapters adapters-present-receptor-type tcr) (Bolotin D A. et al., Nat Methods. 2015; 12:380-1). Resulting TCR outputs were then converted to VDJtools format using the VDJtools (v.1.2.1) software suite (Shugay M. et al., PLOS Comput Biol. Public Library of Science; 2015; 11:e1004503) for downstream analysis. To characterize the bulk TCR repertoires from the signature sort experiments in the context of the post-infusion single-cell TCR data, exact nucleotide matches in the CDR3 regions between bulk 13 chain sequences and single-cell 13 chain sequences were identified, using the most highly expressed 13 chains in cases where multiple betas were identified in an individual cell. After identifying exact 13 chain matches between the bulk GMP data and post-infusion single-cell data, those matches were used to infer the future transcriptional clusters of the 13 lineages for each sample, which were represented in terms of proportions of the bulk 13 sequences with exact matches. The log₂ fold changes between the two sort schemes was then calculated.

### Surface Phenotyping and Intracellular Cytokine Staining of SJCAR19 GMP and Post-Infusion Samples

Cryopreserved aliquots of the GMP cell product (pre-infusion) or peripheral blood samples collected at peak expansion after CAR T cell infusion (post-infusion) for matched donors (when available; patients 16-23) were thawed at 37° C., suspended in RPMI 1640 supplemented with 10% heat-inactivated human AB serum (Gemini Bio-Products, Cat #100-512), 1% non-essential amino acids (Gibco, Cat #11140-050), 1 mM sodium pyruvate (Gibco, Cat #11360-070), and 100 U/mL penicillin-streptomycin and allowed to rest for 12 hours at 37° C. and 5% CO₂. Pre- or post-infusion samples were plated at 3.0×10⁵ cells/well in a 96-well U-bottom plate. While post-infusion samples were cultured alone, pre-infusion samples were co-cultured 2:1 (sample:tumor) with 1.5×10⁵ cells/well of CD19+ tumor, BV-173 (Accession #CVCL_0181), (positive stimulation) or CD19 KO BV-173 (52, 53; unstimulated) at 37° C. and 5% CO₂ Mycoplasma testing for both cell lines was conducted using the MycoAlert Assay (Lonza) in March 2021, and authentication of both cell lines was conducted in March 2022 by the American Type Culture Collection (ATCC.org) using short tandem repeat markers. Cell lines were passaged three times prior to use in these experiments. At the 12-hour time point, 1×PMA/ionomycin (eBioscience, Cat #00-4970-93) was added to positive control wells and GolgiPlug (BD, Cat #555029) and GolgiStop (BD, Cat #554724) were added at 1:1000 to all wells. Cells were incubated for an additional 12 h (24 hours total), washed twice with FACS buffer (1×PBS, 2% FBS, 1 mM EDTA), resuspended in 50 μL FACS buffer containing 5 μL human Fc-block (Biolegend, Cat #422302), and blocked for 10 min at 4° C. Cells were surface stained in an additional 50 uL FACS buffer containing 1 μL Ghost Dye Violet 510 Viability Dye (Tonbo Biosciences, Cat #13-0870-T100) and a cocktail of fluorescent anti-human antibodies: CXCR3-BUV563 (BD, Cat #741406), CX3CR1-BUV661 (BD, Cat #750690), CXCR6-BUV805 (BD, Cat #748448), CD27-SuperBright 436 (ThermoFisher, Cat #62-0271-82), TIM3-BV480 (BD, Cat #746771), CD8-BV570 (Biolegend, Cat #301038), CD69-BV605 (Biolegend, Cat #310938), CD62L-BV650 (Biolegend, Cat #3014832), PD1-BV711 (Biolegend, Cat #329928), CD3-BV750 (Biolegend, Cat #344846), CD4-BB515 (ThermoFisher, Cat #566912), CD74-PerCP/Cy5.5 (Biolegend, Cat #357608), TIGIT-PerCP/eFluor710 (ThermoFisher, Cat #50-245-943), CD49d-PE/Dazzle 594 (Biolegend, Cat #304326), CXCR4-PE/Cy5 (Biolegend, Cat #306508), CD52-PE/Cy7 (Biolegend, Cat #316012), LAG3-Alexa Fluor 647 (Biolegend, Cat #369304), and CCR5-Alexa Fluor 700 (Biolegend, Cat #359116) for 30 min at 4° C. Cells were washed twice with FACS buffer and prepared for intracellular and intranuclear staining using the FoxP3/Transcription Factor Staining Buffer protocol (ThermoFisher, Cat #00-5523-00: cells were resuspended in 200 μL Fixation/Permeabilization buffer, incubated 30 min at RT then washed twice in 200 uL 1×Permeabilization buffer. Co-cultures were stained with 100 uL CAR Detection Reagent (Miltenyi Biotec, Cat #130-115-965) in 1×Permeabilization buffer for 10 min at RT, washed in in 200 uL 1× Permeabilization buffer and stained for intracellular cytokines for 30 min at 4° C. with a 100 uL cocktail of 1×Permeabilization buffer and anti-human antibodies including GzmB-BV421 (Biolegend, Cat #396414), IFNγ-BV785 (Biolegend, Cat #502542), GzmK-FITC (Santa Cruz Biotechnology, Cat #sc-56125), TOX-PE (ThermoFisher, Cat #50-245-516), and Biotin-APC (anti-CAR secondary antibody; Miltenyi Biotec, Cat #130-111-068). Cells were washed twice with FACS buffer, suspended in 120 uL FACS buffer, and analyzed by flow cytometry on a Cytek 5-laser Aurora spectral flow cytometer using SpectroFlo v2.2 software (Cytek) and analyzed using FlowJo v10.7.1 software (TreeStar).

Supplementary Clinical Information on Patients Enrolled on SJCAR19 (a Phase III Study Evaluating CD19-Specific Engineered Autologous T Cells in Pediatric and Young Adult Patients ≤1239 21 Years of Age with Relapsed or Refractory CD19+ Acute Lymphoblastic Leukemia; 1240 NCT03573700)

The clinical results of patients 1 to 12 have been reported elsewhere (Talleur et al; Blood Adv 2022). In addition, the clinical course of patients 1 and 9 have been 1243 published (Hines et al; Br J Haematol. 2021 August; 194(4):701-707. onlinelibrary.wiley.com/doi/full/10.1111/bjh.17662). For patient 0, a GMP CD19-CAR T cell product was manufactured, but patient 0 did not proceed to the treatment portion of the clinical study due to poor clinical status in the setting of rapidly progressive B-ALL. Patients 13-23 were enrolled on the Phase II portion of the clinical trial and received protocol prescribed treatment with lymphodepleting chemotherapy (fludarabine/cyclophosphamide) followed by infusion of CD19-CAR T cells (3×10\hat{ }6 CAR+ T cells/kg). These 11 patients were majority male (n=6), with a median age of 6.5 years old (range 2-9 years) at time of infusion. Pre-treatment disease burden in the marrow ranged from 0-61% blasts by morphology; none had detectable leukemia in the cerebrospinal fluid. The outcome of these patients was similar to the first 12 patients reported in our publication (Talleur et al; Blood Adv 2022): CAR T cell infusions were well tolerated with a low incidence of both cytokine release syndrome (any grade, n=7) and immune effector cell-associated neurotoxicity syndrome (ICANS; n=1). Ten out of 11 patients (patients 13 to 20 and 22 to 23) achieved a complete response (CR) at 4 weeks post infusion, of which two were MRD (measurable residual disease) positive. As with the initial patient cohort, patients who achieved a CR and proceeded to consolidative allogeneic HCT had excellent outcomes, with 5 out of 6 patients being alive and in CR at time of last follow-up.

### Statistical Analyses

The differential expression analysis across clusters in a pairwise manner, GZMK expression difference between cluster 3 and 8, and transcriptional profile of the CAR product CD8+ lineages that end in clusters 3 and 8 and compared them to all other CD8+ CAR T cell product were performed based on non-parameteric Wilcoxon rank sum test by Seurat package (version 4.0.1) on R (version 4.1.0). The p-values were corrected for multiple testing with the Bonferroni procedure and used the genes with the adjusted p-value<0.05 as statistically significant ones.

For evaluating a difference between average clone sizes in persistent clonal lineages compared to clonotypes that were only observed once, Kolmogorov-Smirnov test was used and p-values were generated based on the test to provide statistical significance.

The pseudotime analysis applied here is Monocle2 (version 2.20.0) which utilizes DDRTree (Discriminative Dimensionality Reduction via learning a Tree). The DDRTree is a reversed graph embedding technique to reduce the data's dimensionality and make the single-cell data into a tree format, so that the trajectories can be revealed and the pseudotime is calculated.

When the classifier for predicting CAR T cell effector in CAR products was built, the top 100 differentially expressed genes between downsampled pre-effector and the other downsampled CD8+ CAR T cell products were selected for training. It was iterated 1,000 times to get a robust result and LOOCV (Leave One Out Cross Validation) approach was used in each iteration.

### Example 1. Pre-Infusion and Post-Infusion CART Cells have Distinct Gene Expression Profiles

To determine features of effective CART cell therapies, a comprehensive gene expression profiling of 15 pediatric patients with B-cell acute lymphoblastic leukemia (B-ALL) (Table 5), who had received autologous T cells expressing a CD19.4-1BBz CAR (CD19-CAR), post lymphodepleting chemotherapy on an investigator-initiated clinical study (NCT03573700) was undertaken, including pre-infusion CAR T cell product (i.e., CAR product) and post-infusion samples obtained from PBMCs and bone marrow aspirates at regular intervals. Single-cell gene expression and T cell receptor (TCR) sequencing were performed on sorted T cell populations in the CAR product and at weeks 1-4, 8, and month 3 and 6 post-infusion. Across all patients and timepoints, 118,749 CAR product CAR T cells and 66,042 post-infusion CAR T cells were sequenced for single-cell gene expression, with an average of 11,549 cells per patient (SD=7,335) and 20,532 cells per time point (SD=37,898). Of note, the month 6 post-infusion time point only consisted of 7 CAR cells, all derived from a single patient (Table 6). CAR T cells from the CAR product represented 6400 of the total CAR T cell population and exhibited on average 3,320 genes per cell (SD=856). Post-infusion CAR T cells were less transcriptionally active, expressing 2,299 genes per cell on average (SD=931).

TABLE 5
Patient demographics

| | | | Prior | | CAR dose | Response | | Total |
|---|---|---|---|---|---|---|---|---|
| | Age at | | antigen | | received | (marrow; 4- | Post- | CAR T |
| Pat | infusion/ | Prior | directed | Disease | (# of | weeks post- | infusion | cells |
| no. | Sex | HCT | therapy | indication | cells/kg) | infusion) | HCT | sequenced |
| 0 | 18/M | Y | B/I/ | Relapse >/3 | N/A | N/A | N/A | 1,341 |
| | | | CD19CAR/ | | | | | |
| | | | CD22CAR | | | | | |
| 1 | 20.4/M | N | B | Relapse 3 | 1 × 10⁶ | NR | N | 1,885 |
| 2 | 18.5/F | N | N | Relapse 2 | 1 × 10⁶ | MRDneg CR | Y | 9,804 |
| 3 | 16.3/M | N | N | Primary | 1 × 10⁶ | MRDneg CR | Y | 8,543 |
| | | | | refractory | | | | |
| 4 | 10.4/M | Y | B | Relapse 2 | 1 × 10⁶ | MRDneg CR | N | 14,738 |
| 5 | 15.4/F | Y | N | Relapse 3 | 1 × 10⁶ | MRDneg CR | N | 23,994 |
| 6 | 1.8/F | Y | B/I | Relapse 2 | 1 × 10⁶ | MRDneg CR | N | 10,902 |
| 7 | 12.4/F | Y | B | Relapse 2 | 3 × 10⁶ | MRDneg CR | N | 7,757 |
| 8 | 15.4/F | N | B | Relapse 2 | 3 × 10⁶ | MRDneg CR | N | 20,569 |
| 9 | 6.2/F | N | N | Relapse 2 | 3 × 10⁶ | MRDneg CR | N | 15,389 |
| 10 | 5.6/F | N | N | Relapse 2 | 3 × 10⁶ | MRDneg CR | Y | 20,622 |
| 11 | 21.8/M | N | N | Relapse 2 | 3 × 10⁶ | MRDneg CR | Y | 22,965 |
| 12 | 12.3/F | N | N | Relapse 1 | 3 × 10⁶ | MRDneg CR | Y | 4,529 |
| | | | | (refractory) | | | | |
| 13 | 8/M | Y | B/CD19CAR | Relapse 1 | 3 × 10⁶ | MRDneg CR | Y | 8,292 |
| 14 | 9/M | N | N | Relapse 2 | 3 × 10⁶ | MRDneg CR | Y | 5,634 |
| 15 | 9/M | N | N | Relapse 1 | 3 × 10⁶ | MRDneg CR | N | 7,827 |
| | | | | (refractory) | | | | |

M = male;
F = female;
HCT = hematopoietic cell transplant;
N = no;
Y = yes;
B = Blinatumomab;
I = Inotuzumab;
MRD = minimal residual disease;
NR = no response;
MRDneg CR = MRD-negative complete response;
NA = not applicable (patient was not infused with CAR product)

TABLE 6

| Patient | Pre-infusion | Post infusion | | | | | | |
|---|---|---|---|---|---|---|---|---|
| no. | GMP Product | Week 1 | Week 2 | Week 3 | Week 4 | Week 8 | Month 3 | Month 6 |
| 0 | 1,341 | | | | | | | |
| 1 | 1,885 | | | | | | | |
| 2 | 8,568 | 261 | 830 | 84 | 61 | | | |
| 3 | 8,222 | 169 | 146 | 1 | 4 | | | |
| 4 | 12,280 | 340 | 1,220 | 459 | 434 | 5 | | |
| 5 | 11,988 | 9,510 | 1,027 | 672 | 321 | 114 | 355 | 7 |
| 6 | 8,461 | 144 | 1,444 | 556 | 279 | 5 | 13 | |
| 7 | 7,607 | 36 | 39 | 26 | 38 | 11 | | |
| 8 | 14,840 | 971 | 1,069 | 1,273 | 2,165 | 251 | | |
| 9 | 11,221 | 136 | 2,974 | 874 | 184 | | | |
| 10 | 10,960 | 1,568 | 4,389 | 1,705 | 851 | 1,149 | | |
| 11 | 12,218 | 2,427 | 1,940 | 5,234 | 1,146 | | | |
| 12 | 4,339 | 190 | | | | | | |
| 13 | 4,819 | | | 3,473 | | | | |
| 14 | | 2,855 | 386 | 2,372 | 21 | | | |
| 15 | | 2,478 | 2,148 | 3,143 | 58 | | | |

Empty boxes indicate that samples were not available for analysis; Patients 0, 1, 12, 13, 14 and 15 were excluded from GMP-post infusions lineage tracing analysis due to data limitations

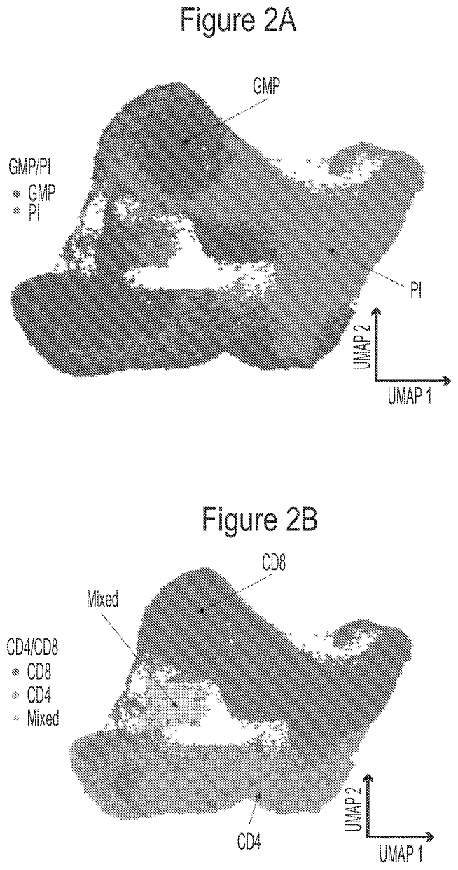

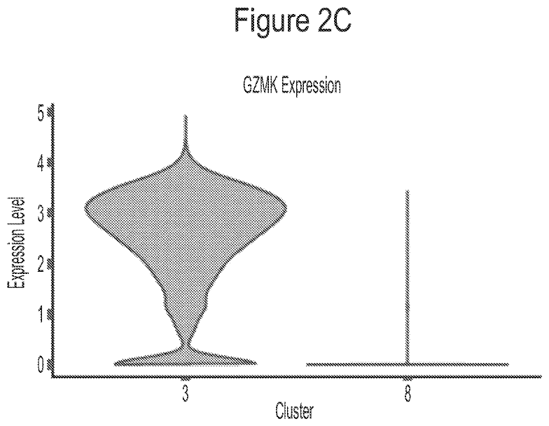

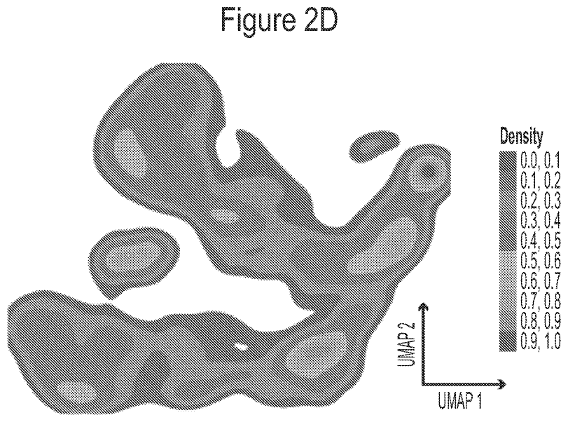

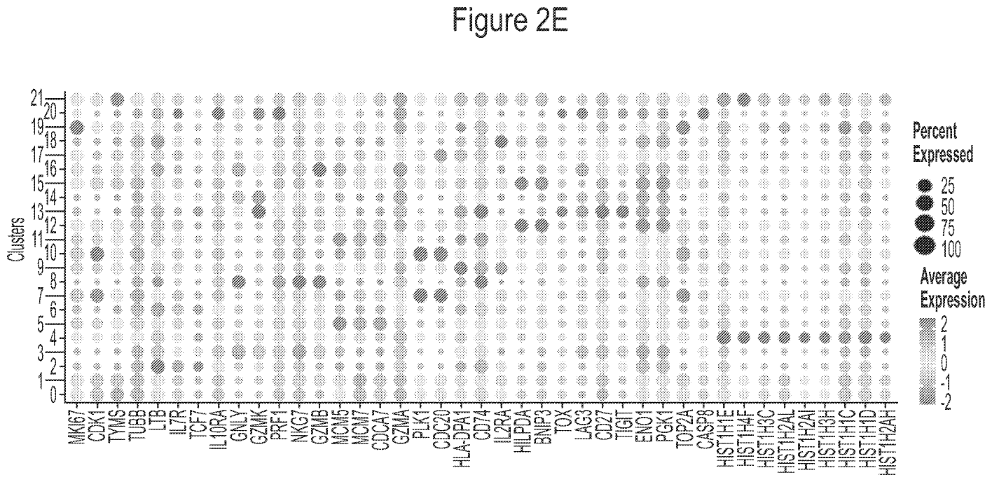

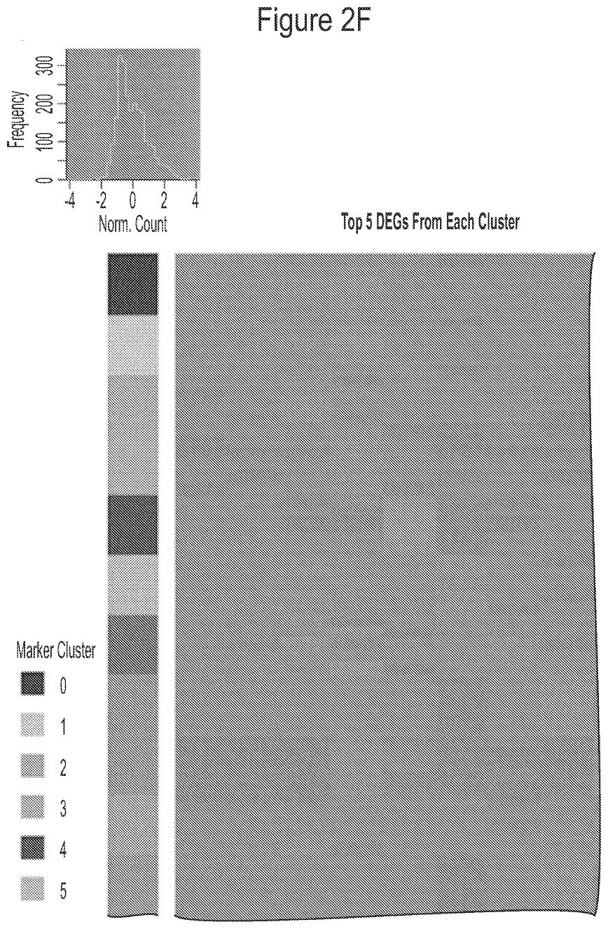

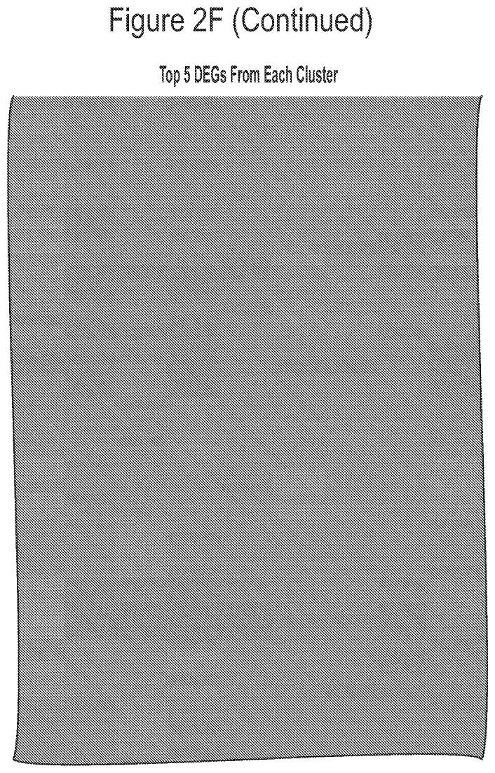

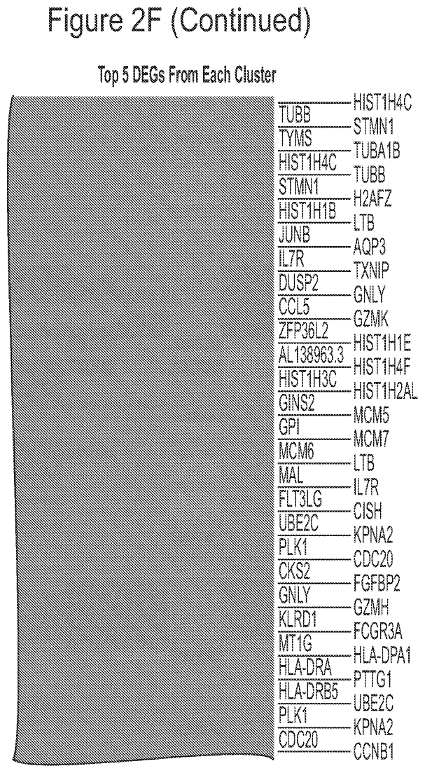

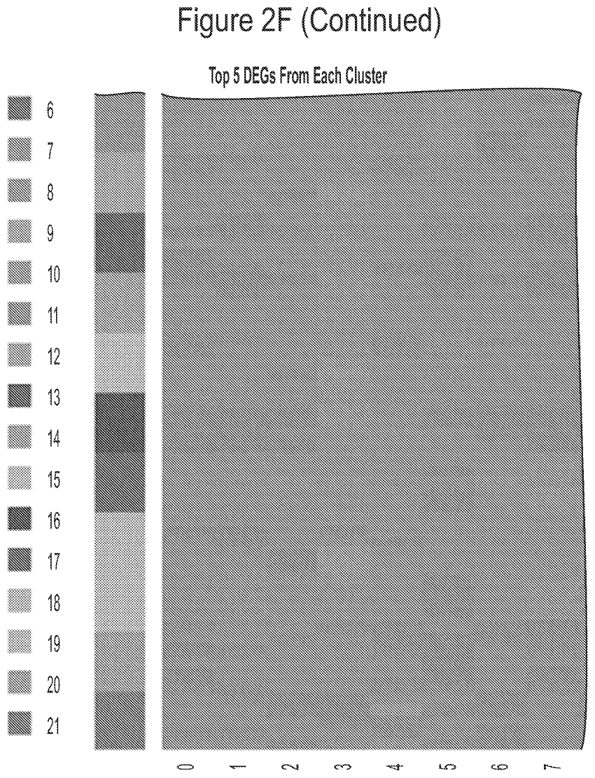

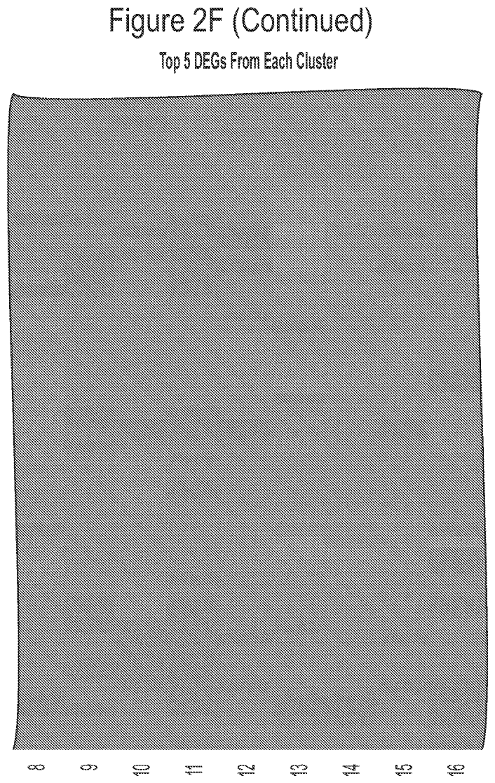

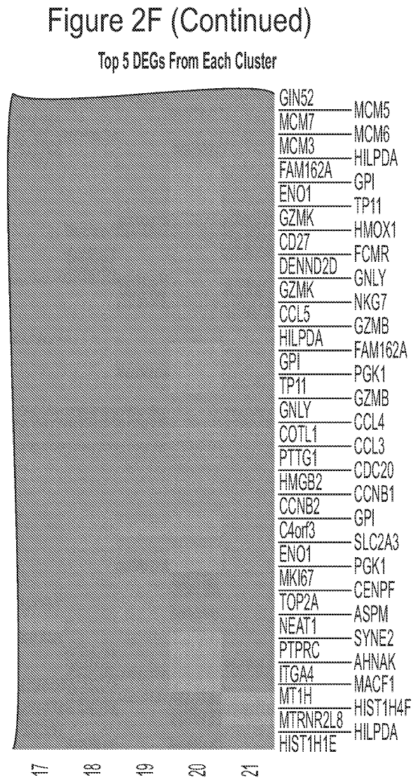

To understand the range of CART cell phenotypes and how these phenotypes change from the pre-infusion product throughout the course of treatment, UMAP dimensionality reduction and clustering analysis was performed based on gene expression profiles (FIG. 1A). Included in this broad analysis were both CAR product and post-infusion CAR T cells, which were generally segregated across the primary axis of variation (FIG. 2A). Unsurprisingly, CD4+ and CD8+ CART cells also separated distinctly, with few clusters shared by both T cell types (FIG. 21B). Differential gene expression analysis across the clusters revealed several distinct T cell functional states. Clusters predominated primarily by CAR product CAR T cells were classified as proliferative with high expression of genes such as MKI67, cell-cycling genes CDK1 and CDC20, DNA replication genes MCM7, TOP2A and TYMS (CD8: clusters 1, 5, 7, 9, 12, 19, 21; CD4: clusters 0, 10, 11, 15; FIGS. 1A-1C). In contrast, a subset with relatively lower proliferation signals but high expression of ENO1, PGK1, and BNIP3 was identified as metabolically active CD4+ CAR product T cells (cluster 18; FIG. 1C).

Note that FIG. 1A includes data from both the CAR product and post-infusion CAR T cells. However, as described infra in Example 4, precursors to cytotoxically effective cells identified in the CAR product were spread across multiple clusters, and as described in Example 5, precursors to cytotoxically effective cells identified in the CAR product were identified based on clonal relation of TCR to clusters 3 and 8, which were identified as representing post-infusion CD8+ functional effector cells. Therefore, including the CAR product data in the clustering algorithm was not necessary to the ultimate identification of precursors to cytotoxically effective cells as outlined in Example 5. Further, it is noted that an alternative clustering algorithm (e.g., a supervised clustering algorithm) may have resulted in precursors to cytotoxically effective cells being clustered together, and those resulting clusters may be mapped to clusters representing post-infusion CD8+ functional effector cells as was attempted in Example 4.

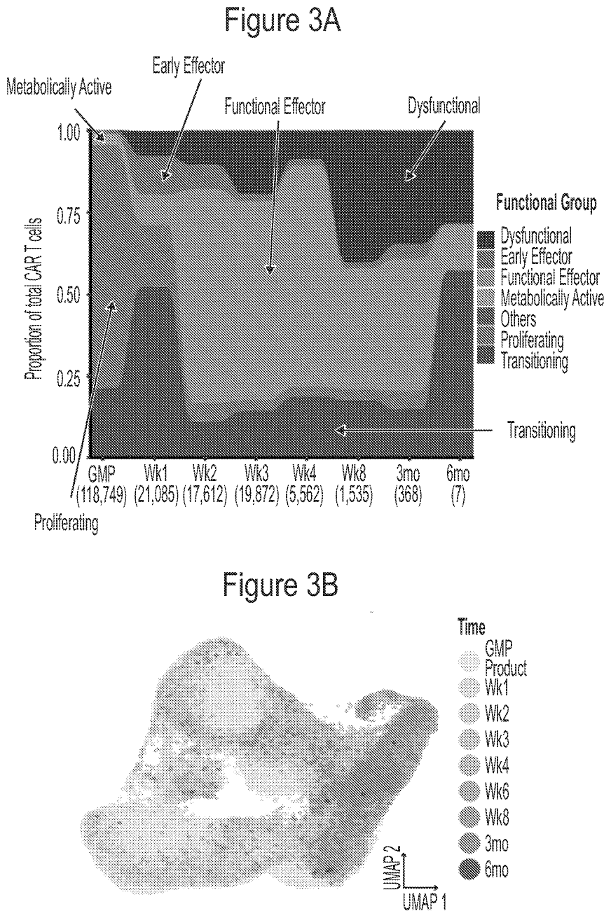

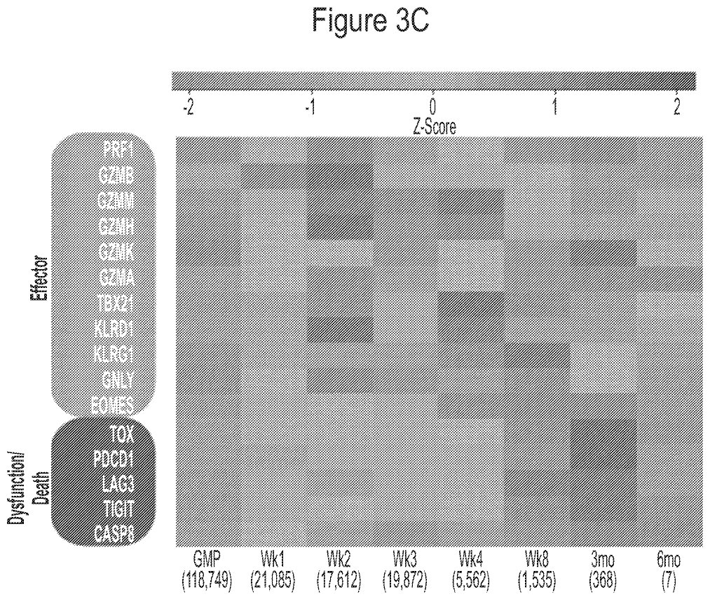

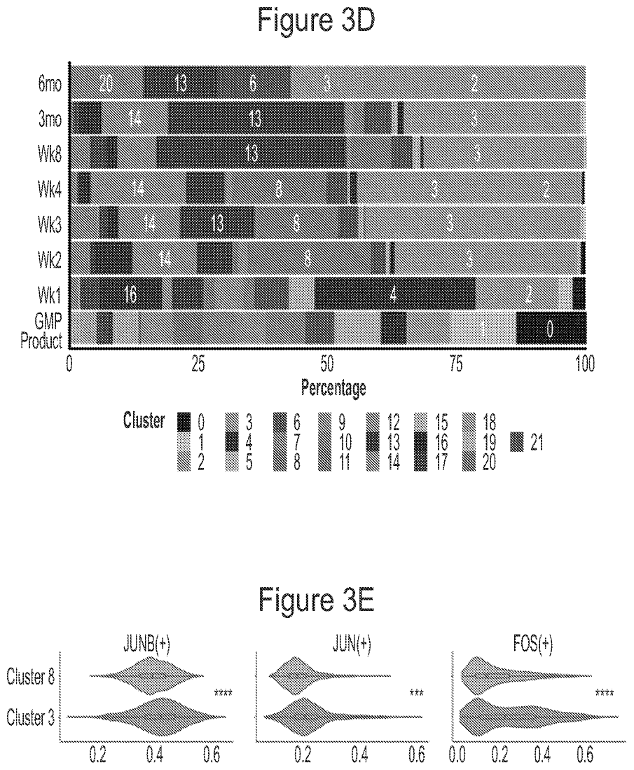

Within post-infusion CAR T cells, several functional effector T cell populations characterized by robust expression of cytotoxic genes GNLY, PRF1, and NKG7 (CD8: clusters 3, 8, and 16; CD4: cluster 14; FIGS. 1B-1C) were observed. Interestingly, expression of GZMK distinguished cluster 3 from cluster 8, the classic effector GZMB-expressing effector population (FIG. 2C). Others have previously suggested that GZMK expressing T cells are a distinct T cell population (Bratke 2005), and it was found herein that this cluster was more heterogeneous in expression space compared to the tightly grouped cells from cluster 8 (FIG. 3A; FIG. 2D). A separate, early functional effector cluster (16) was distinguished by upregulation of MCM5, indicating a maintenance of the proliferation signals seen in CAR T cells product despite increased effector T cell genes (FIG. 2E). Two dysfunctional CAR T cell post-infusion populations unique to CD8s, clusters 13 and 20, were also identified. High expression of a suite of inhibitory markers within cluster 13, including TOX, LAG3, and TIGIT, suggested T cell exhaustion (FIGS. 1A-C). In contrast, cluster 20 CAR T cells exhibited apoptotic processes, indicated by downregulation of genes needed in translation, ribosomal subunit genes RLP30 and RPL32, and high expression of CASP8 (FIG. 1B). Despite the presence of substantial clusters characterized by proliferating CD4s within the CAR product, CD4+ CAR cells made up only 25.7% of post infusion cells profiled. Similarly, the size of the CD4 effector compartment was much smaller than that of the CD8 CAR T cells post infusion, likely stemming at least in part from the presence of a proliferating CD4 cluster (9) characterized by early dysfunction.

Although most transcriptional clusters were strongly associated with either pre- or post-infusion samples, some populations (CD8: 6 and 17; CD4: 2; FIG. 2E) instead exhibited a putative transitional state between the proliferative signature associated with CAR T cells product and the cytotoxic effector differentiated state of the post-infusion CAR T cells. These transitional phenotypes were identified by the joint expression of several cytotoxic genes, including LTB (clusters 2 and 6), GZMA (cluster 6), GZMK (cluster 17), and PRF1 (cluster 6), in addition to the expression of proliferation signals CDC20, PLK1, and MKI67 (FIG. 2E). Lastly, a putative hybrid population, cluster 4, was identified that contained both CAR product and post-infusion cells with mixed CD4+ and CD8+ T cell subsets.

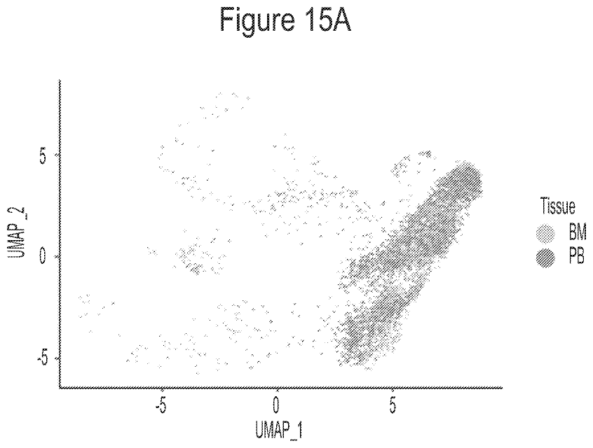

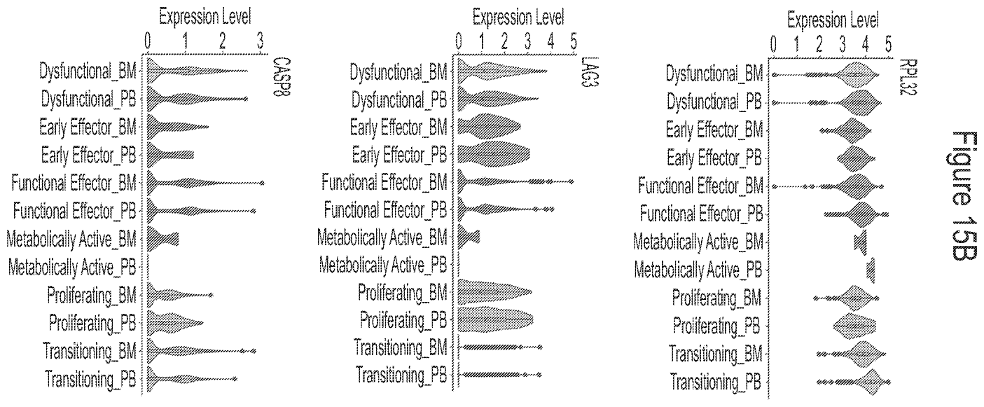

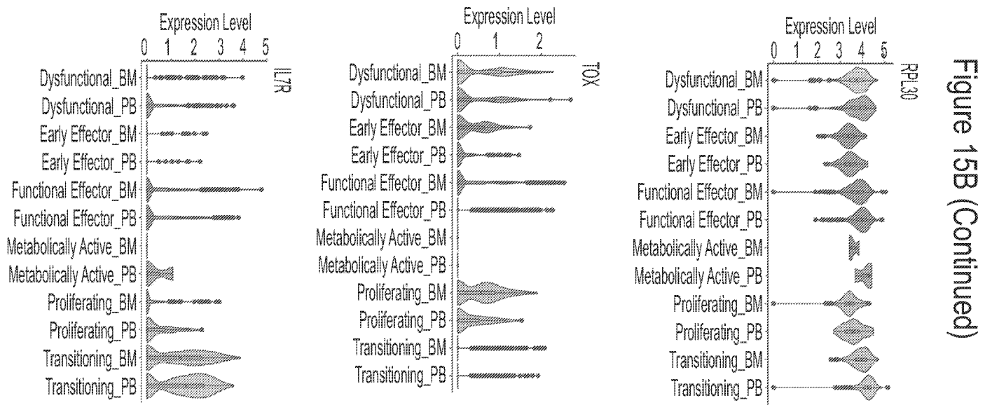

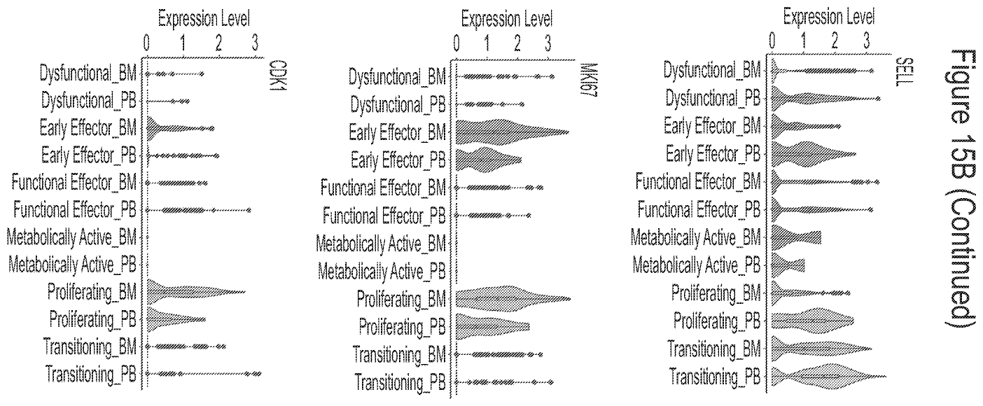

Overall, assessments of variation in gene expression allowed identification of distinct transcriptional programs between CAR T cells product and post-infusion CAR T cells. Signals associated with initiation and maintenance of the complex pathways involved in T cell proliferation were evident within both CD4+ and CD8+ CAR cells product, while post-infusion CAR cells demonstrated signs of differentiation into effector T cells displaying potent cytotoxic transcriptional profiles. CAR T cells obtained from bone marrow aspirates at 4 weeks and 3 months post infusion were largely comparable to those from the periphery at the same study time points (FIG. 15A), though some annotated subsets differed somewhat in degree of expression for key annotation markers (FIG. 15B).

### Example 2. Post-Infusion CAR T Cells Become More Effector-Like Over Time

Having established that CAR product and post-infusion CAR T cells encompass several distinct transcriptional subsets, the proportion of post-infusion CAR T cells that fit within these broad transcriptional categories was next determined. Starting at week 2 post-infusion, the majority of CAR T cells could be attributed to functional effector subsets (CD8: clusters 3 and 8; CD4: cluster 14) (FIG. 3A). Notably, this week-2 time point also coincided with the point of peak expansion in 8 of the 12 responding patients with available qPCR assays of CAR abundance (Tables 7 and 8). Because cytotoxic effectors predominated the post-infusion CAR cell functional groups, it was sought to characterize the kinetics of CAR effector differentiation by assessing variation in the transcriptional signatures across post-infusion timepoints. To do this, post-infusion cells were considered based on their sampling time point but visualized in UMAP space. As expected, cells obtained from earlier time points tended to cluster near cells from the CAR products, whereas cells from later time points were more enriched near the bulk of the post-infusion populations (FIG. 3B). Next, the development of the effector signature across CAR product and post-infusion timepoints was characterized. Compared to CAR cells in the CAR product and at week 1 post-infusion, genes associated with the functional effector signature were most highly expressed starting at week 2 (FIG. 3C). This correlates with a dramatic relative expansion of the populations identified as functional effectors (CD8: 3 and 8; CD4:14) at week 2, as they represented a total of 65% of week 2 CAR cells (FIG. 3D). Interestingly, the CD8+ GZMK expressing functional effector population (cluster 3) was maintained throughout the remainder of the sampling window and constituted a sizable 27.7% of CAR cells at month 3 post-infusion. In contrast, the GZMB expressing effector group was lost after week 8, concurrent with the loss of GZMB expression at week 8 across all clusters (FIG. 3C). Despite CD4+ CAR cells being the minority in the post-infusion CAR compartment, the proportion of CD4+ cytotoxic effectors (cluster 14) was fairly consistent throughout much of the study period and encompassed 12.8% of all CAR cells at month 3 after infusion (FIG. 3D).

TABLE 7
CD19 CAR/μg of DNA.

| Patient no. | GMP | Week 1 | Week 2 | Week 3 | Week 4 | Week 8 | Month 3 | Month 6 |
|---|---|---|---|---|---|---|---|---|
| 1 | 69914 | 3114 | 199054 | 13744 | 1145 | | | |
| 2 | 138709 | 13912 | 42149 | 8992 | 1842 | 83 | | |
| 3 | 261689 | 61777 | 10100 | 681 | 48 | 46 | | |
| 4 | 61481 | 3745 | 288670 | 38325 | 7212 | 472 | 204 | |
| 5 | 68893 | 224445 | 169344 | 7013 | 3937 | 4308 | 9691 | 0 |
| 6 | 96171 | 1011 | 167092 | 47074 | 19005 | 1094 | 139 | 32 |
| 7 | 68833 | 657 | 565 | 4806 | 1686 | 159 | | |
| 8 | 1664810 | 46046 | 142422 | 55085 | 16162 | 3763 | | |
| 9 | 935072 | 4203 | 268449 | 301705 | 218312 | | | |
| 10 | 787811 | 356424 | 68633 | 15701 | 885 | 8045 | | |
| 11 | | 8071 | 64212 | 36722 | 5315 | | | |
| 12 | 902010 | 5835 | | | | | | |
| 13 | 896089 | 6403 | 2193043 | 341921 | 168462 | | | |
| 14 | | 87863 | 3635 | 22611 | 119 | | | |
| 15 | | 18532 | 15465 | 3786 | 292 | 523 | | |

Empty boxes indicate time points with unavailable data

TABLE 8
CD19 CAR/ml of blood.

| Patient no. | GMP | Week 1 | Week 2 | Week 3 | Week 4 | Week 8 | Month 3 | Month 6 |
|---|---|---|---|---|---|---|---|---|
| 1 | | 9341 | 82939 | 6414 | 6677 | | | |
| 2 | | 296787 | 561988 | 149865 | 51570 | 1374 | | |
| 3 | | 947250 | 254181 | 16461 | 958 | 1003 | | |
| 4 | | 24967 | 2165026 | 421573 | 121406 | 3801 | 4209 | |
| 5 | | 1122227 | 254016 | 31556 | 36746 | 114889 | 174431 | 0 |
| 6 | | 50700 | 3369685 | 894403 | 376926 | 17141 | 2970 | 2905 |
| 7 | | 7561 | 16755 | 76891 | 8432 | 872 | | |
| 8 | | 314646 | 569687 | 321331 | 91583 | 89693 | | |
| 9 | | 268449 | 5732386 | 2328662 | 339582 | | | |
| 10 | | 1960334 | 2024664 | 578331 | 27864 | 139449 | | |
| 11 | | 236747 | 2825332 | 1621885 | 168308 | | | |
| 12 | | 66126 | | | | | | |
| 13 | | 49091 | 1754431 | 4843874 | 1740770 | | | |
| 14 | | 1640116 | 69673 | 471052 | 2238 | | | |
| 15 | | 395350 | 520669 | 81407 | 7298 | 10025 | | |

Empty boxes indicate time points with unavailable data

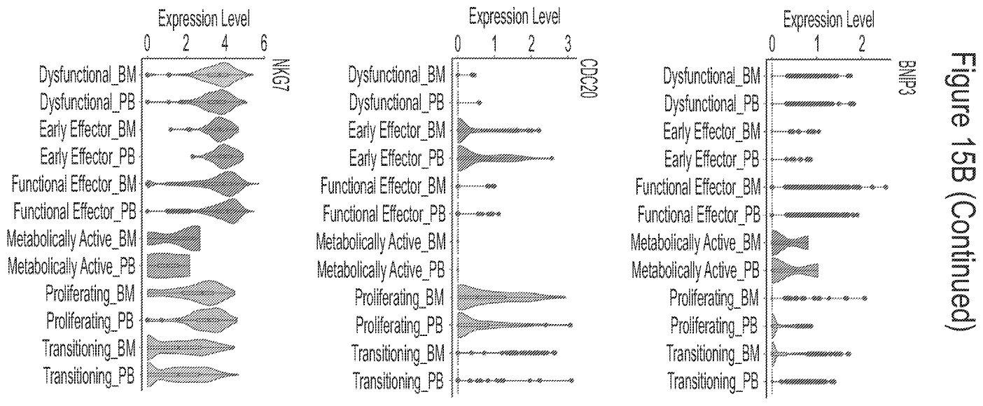

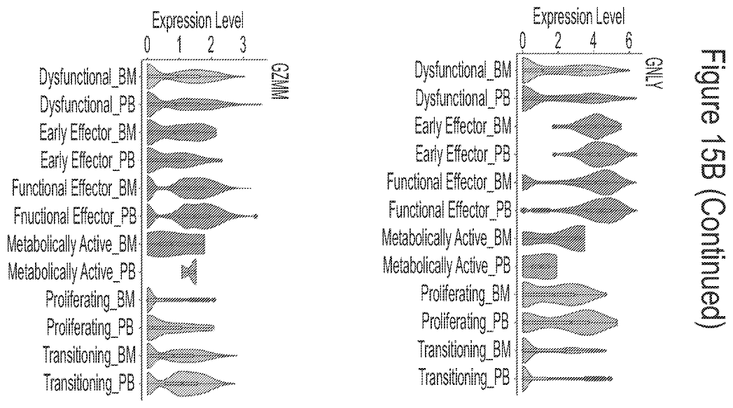

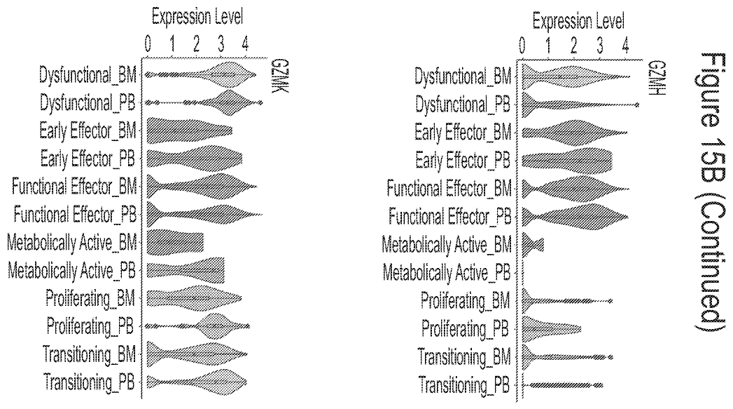

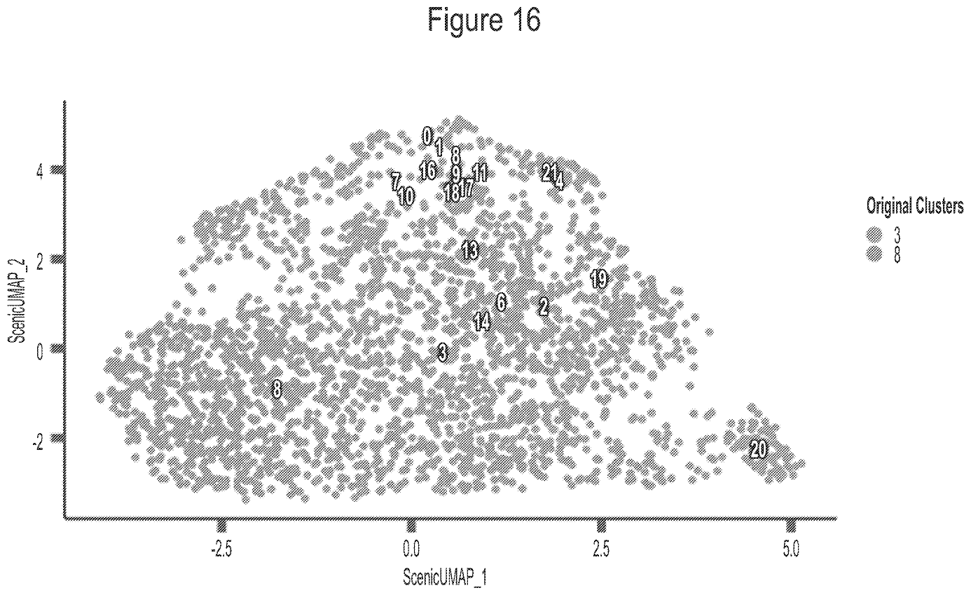

To better understand potential differences between cytotoxic CD8+ clusters 3 and 8 and why their relative abundances might fluctuate over time, differential expression analysis and regulatory network inference (Aibar S. et al., Nat Methods. Nature Publishing Group; 2017; 14:1083-6) was performed next, specifically contrasting these two transcriptionally defined populations. In comparison to cluster 8, cluster 3 expressed significantly higher levels of JUN, JUNB, and FOS, which are members of the AP-1 transcription factor complex known to positively impact cellular proliferation through regulation of cell cycling proteins (Angel P et al., Biochim Biophys Acta BBA—Rev Cancer. 1991; 1072:129-57; Halazonetis T D et al., Cell. 1988; 55:917-24; Curran T et al., Cell. 1988; 55:395-7; Sassone-Corsi P et al., Nature. Nature Publishing Group; 1988; 336:692-5; Shaulian E et al., Nat Cell Biol. Nature Publishing Group; 2002; 4:E131-6). Analysis of inferred coregulated gene networks on a downsampled subset of the post-infusion data confirmed the upregulation of JUN(+), JUNB(+), and FOS(+) regulons in cluster 3 compared to cluster 8 (FIG. 3E), further indicating the enrichment of this transcription factor program within this cluster. Moreover, AP-1 signaling, and c-Jun specifically, has been shown to be a critical element of T cell populations that do not proceed towards a terminally exhausted phenotype (Martinez G J et al., Immunity. 2015; 42:265-78; Seo W et al., Exp Mol Med. Nature Publishing Group; 2021; 53:202-9; Lynn R C et al., Nature Publishing Group; 2019; 576:293-300.). Concordant with broader gene expression analyses (FIG. 1A), GZMK-GZMB+ cluster 8 also seemed to exhibit less variation in inferred regulatory network expression, again suggestive of a more focused transcriptional state than observed in the more broadly variable cluster 3 (FIG. 16).

Dysfunctional T cell responses, including exhaustion, can arise in some cases of prolonged activation and effector function (Wherry and Kurachi 2015). It was, therefore, investigated whether markers of T cell exhaustion and cellular death, identified within dysfunctional clusters 13 and 20, appeared at a time congruent with extended effector function. As early as week 1, markers often associated with exhaustion and apoptosis were observed in post-infusion CAR cells; however, later timepoints (week 8 and month 3) were more enriched for exhaustion and apoptotic genes (FIG. 3C). This is supported by the timepoints in which the exhausted CD8+ T cell cluster (cluster 13) appears after infusion, as it constituted only a minor proportion of the population at week 1 (6.3% of week 1 CAR cells), increased to 14.4% at week 3, and made up 36.8% and 34.2% at week 8 and month 3, respectively (FIG. 3D). GZMK was also expressed highly at month 3 post-infusion, concordant with an increase in exhaustion signals. This signature has been associated with precursor exhausted T cells (Tpex) (Galleti and Zheng). The dying population (cluster 20) was especially enriched at week 3 and month 6 post-infusion, yet this phenotype was also present in earlier post-infusion timepoints (FIG. 3D). Although the enrichment of dysfunctional signals later suggests that a subset of dysfunctional CAR cells arise as a result of chronic effector function, evidence of dysfunction appearing as early as week 1 post-infusion may indicate that some cells develop their dysfunctional state almost immediately after infusion.

### Example 3. Pseudotime Identifies Two Distinct Trajectories for Post-Infusion CAR T Cell Differentiation

Comparing gene expression clusters to sample kinetics demonstrated a general trend towards effector differentiation, which accelerated at 2 weeks after infusion. However, these data also revealed surprising heterogeneity in gene expression profiles, with dysfunctional cells present even at the very earliest time points after infusion. To explore the relationship between cell fates and differentiation states, in particular the precursors and offspring of potent functional effectors (CD4: clusters 14; CD8: clusters 3 and 8) and dysfunctional cells (CD8: clusters 13 and 20), pseudotime analysis was utilized (on a downsampled subset of the data; see Materials and Methods), where each cell is assigned a putative degree of progression along an inferred trajectory. Cells that fell within a specific area of the pseudotime were grouped into states.

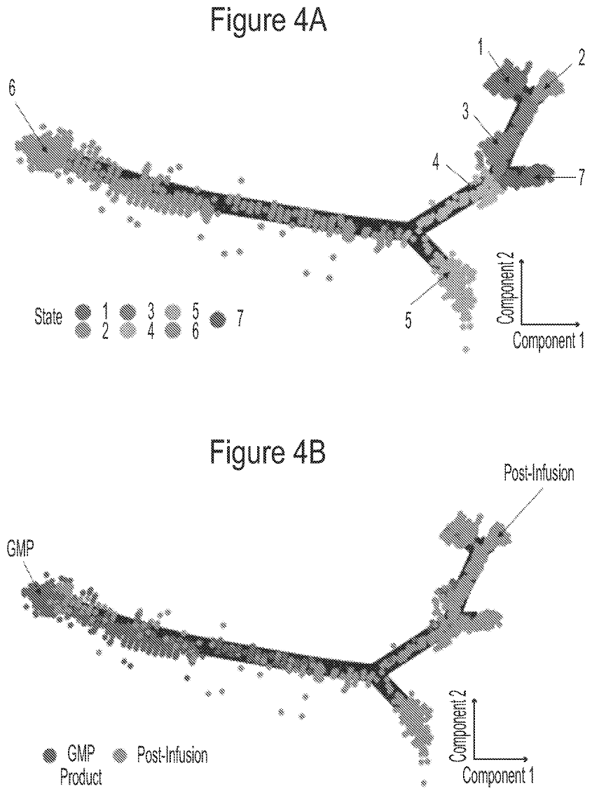

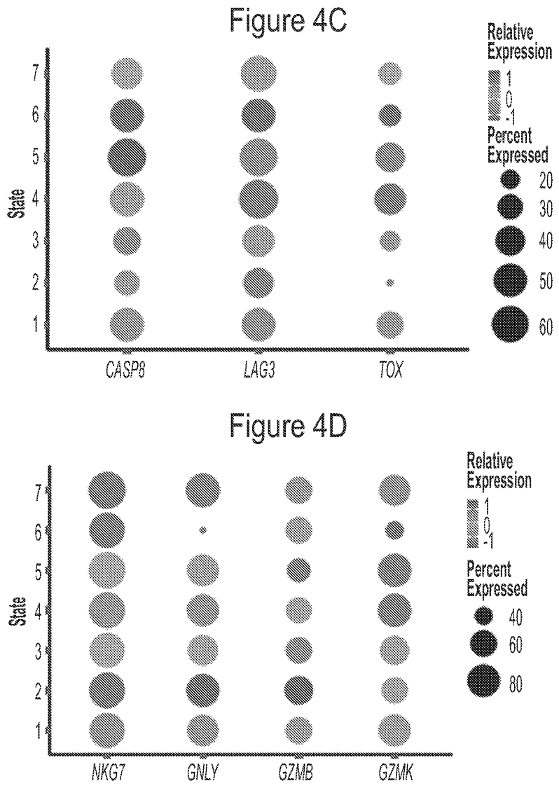

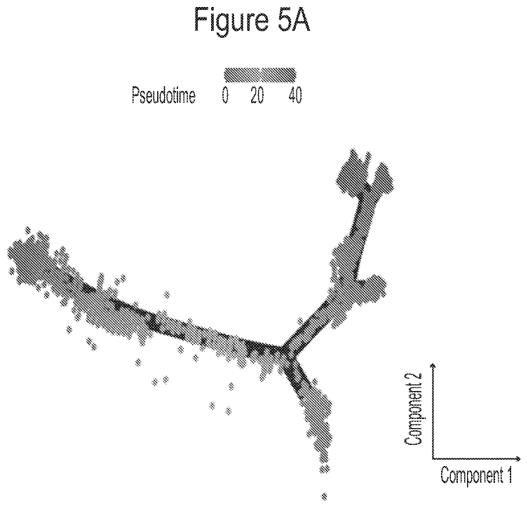

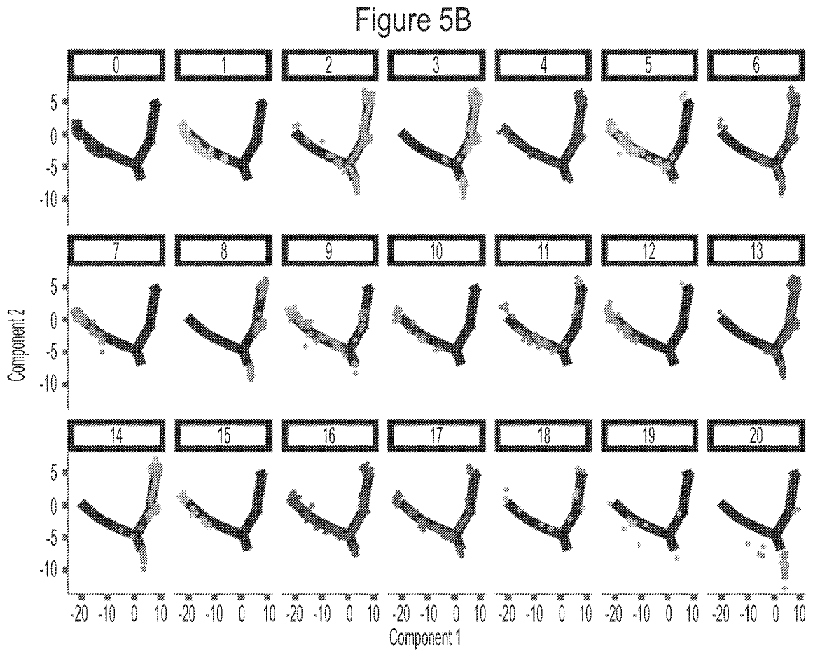

CAR T cells from pre- and post-infusion samples clustered along 7 distinct states (FIG. 4A), with the root of the trajectory in state 6 (FIG. 5A) Unsurprisingly, the majority of CAR product CART cells fell within the root of the pseudotime trajectory, while post-infusion cells predominated among the branching trajectories (FIG. 4B). As cells progress along the pseudotime trajectory, one group (state 5) splits into a distinct differentiation pathway from the others. Because state 5 arises earlier in pseudotime than the other states, state 5 was classified as “early” and states 1, 2, 3, and 7 as “late.” Of note, dysfunctional cluster 13 spanned both the early and late transcriptional states, whereas dysfunctional cluster 20 was primarily confined to state 5. Due to cluster 20's association with cellular death, the expression of the apoptotic gene CASP8 across all states was evaluated (FIG. 5B). Comparatively, expression of CASP8 was highest in state 5 relative to all other states (FIG. 4C). Thus, the pseudotime analysis indicated that a subset of cells after infusion directly acquire a dysfunctional phenotype and proceed to cluster 20 (state 5), which likely results in cell death. In contrast, another trajectory terminated in a potent effector phenotype (states 2, 4, and 7; FIG. 4D), while a subset of those proceed on to exhaustion, as characterized by higher relative expression of TOX and LAG3 (state 4; FIG. 4C). These interpretations broadly concur with the present traditional gene expression analysis that indicated dysfunctional phenotypes developed in a subset of cells early after infusion.

### Example 4. The TCR Serves as a Barcode for CART Cell Lineages

While pseudotime is a useful tool to infer lineage relationships and identify potential differentiation branch points, to understand what transcriptional signatures give rise to the desired effector CAR T cells, a method to definitively track these lineages between pre- and post-infusion was needed. The endogenous T cell receptor (TCR) is an optimal marker of T cell lineages, as it remains static throughout differentiation and is passed to each daughter cell during T cell proliferation, a process known as clonal expansion. It is estimated that most paired up TCRs are represented in the naive human repertoire only a small number of times, generally once (Arstila, Robins, Qi). Therefore, clonal expansions, defined by cells with at least one matching α- and one matching β-chain, in the effector and memory repertoires have a high likelihood of being descended from the same parental clone. Thus, it was hypothesized that the endogenous TCR in transduced cells could be utilized as a lineage barcode for CART cells after product generation and infusion.

To test whether CAR clonotypes across multiple timepoints could be tracked, the number of unique clones in CAR product samples and at each post-infusion time point were first quantified. Targeted cDNA enrichment of the complementarity-determining region 3 (CDR3) of both α and β subunits of the TCR yielded a total of 153,853 unique αβ pairs. Cells with a given αβTCR appearing at more than one time point were defined as “lineages”. Although the lineages identified by this classification are persistent across timepoints, it is important to note that these lineages were undersampled due to the fact that the infused cells within a patient cannot exhaustively sampled; importantly in subsequent analyses, this undersampling biases the results towards the null hypothesis that persistent and non-persistent lineages are similar.

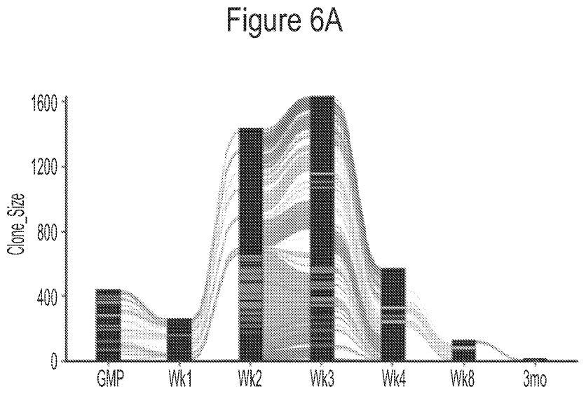

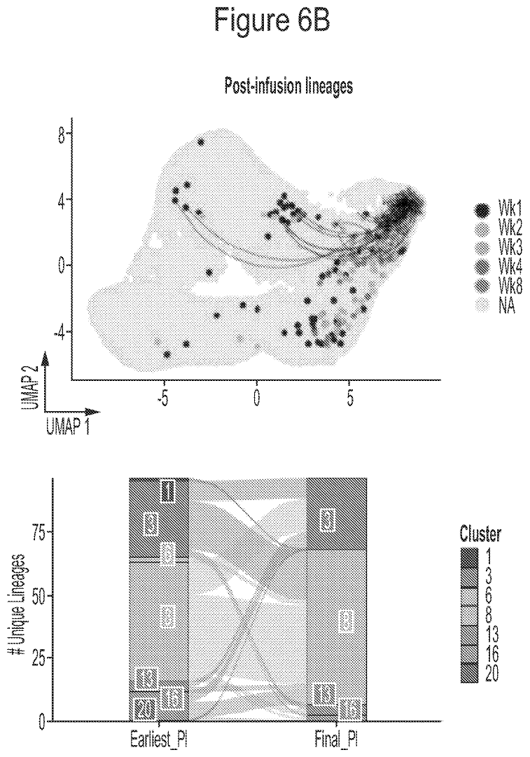

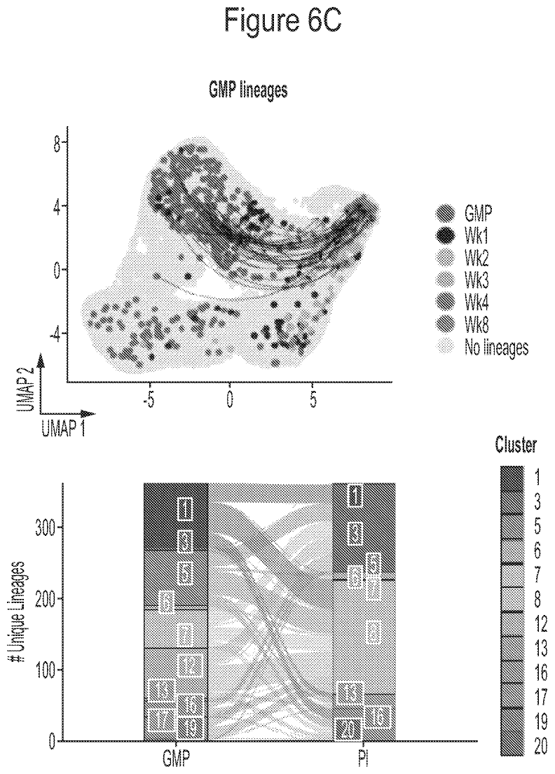

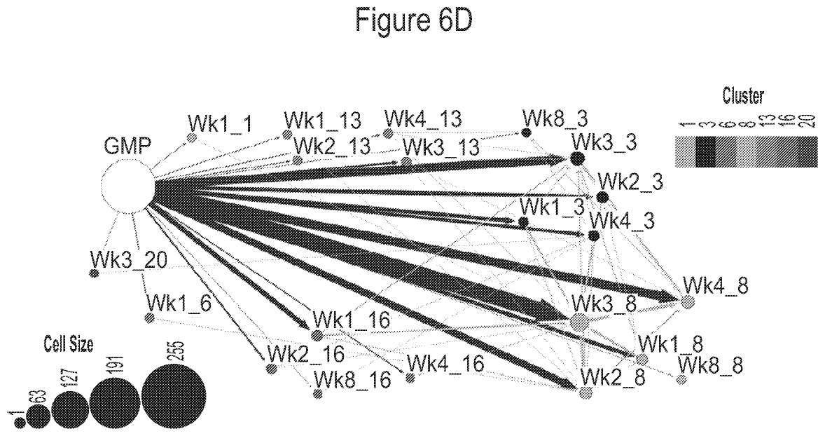

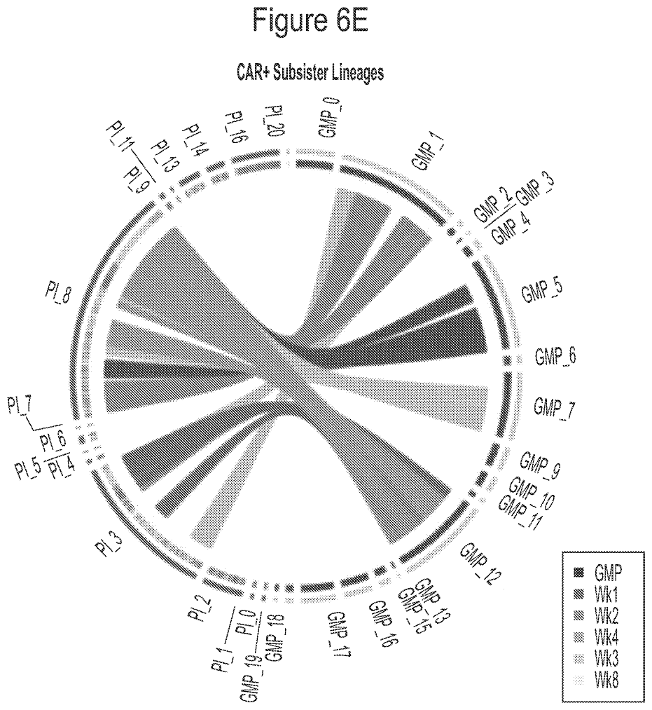

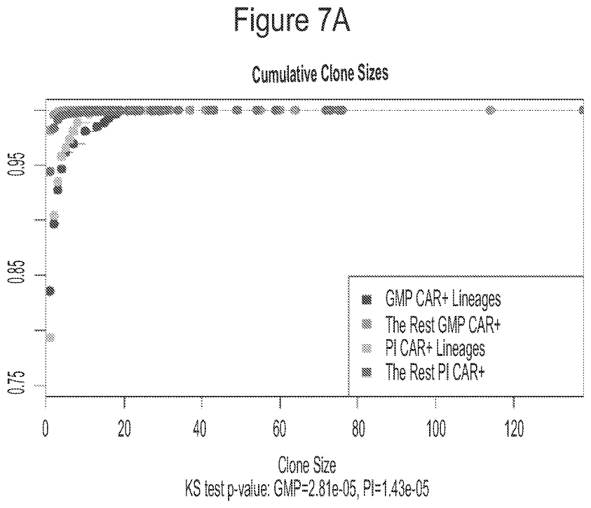

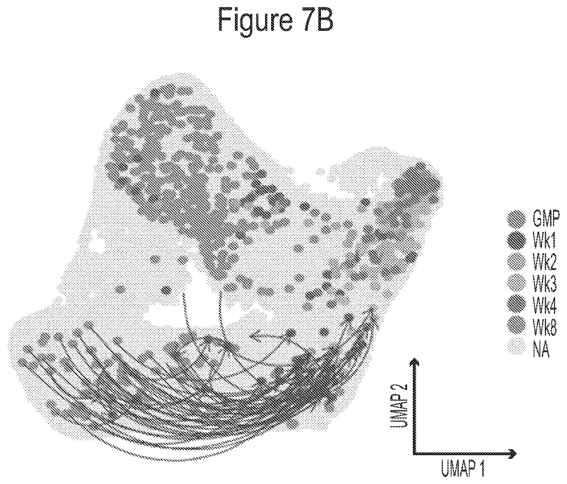

In 10 out of 15 infused patients, multiple lineages originating in the CAR product with a range of 4 to 125 (FIG. 6A; Tables 6 and 9) were tracked. Overall, investigations into relative clonal dynamics of CAR T cells over time uncovered considerable diversity in the CAR product. After infusion, evidence of clonal focusing (clone size>10) occurred in 7 patients (Table 10). A range of clone sizes among CAR cells across the post-infusion time points was observed. For instance, 44,981 TCRs assayed post-infusion were only observed once. However, 2,620 TCRs more than once but in ≤10 cells were also detected. Analyzing these data across all patients, a difference was observed between average clone sizes in persistent clonal lineages compared to clonotypes that were only observed once (FIG. 7A), with persistent lineages having larger overall clone sizes. This suggests that these lineages represent expanded or expanding populations.

TABLE 9
Assessing performance of different
strategies to define CAR lineages

| Patient | α only | β only | one α & one β | Strict |
|---|---|---|---|---|
| 0 | N/A | N/A | N/A | N/A |
| 1 | N/A | N/A | N/A | N/A |
| 2 | 42 | 13 | 9 | 7 |
| 3 | 4 | 1 | 0 | 1 |
| 4 | 119 | 87 | 74 | 65 |
| 5 | 271 | 75 | 32 | 27 |
| 6 | 133 | 96 | 100 | 81 |
| 7 | 13 | 13 | 11 | 11 |
| 8 | 367 | 114 | 70 | 62 |
| 9 | 228 | 52 | 41 | 29 |
| 10 | 566 | 242 | 163 | 139 |
| 11 | 658 | 452 | 385 | 361 |
| 12 | 1 | 0 | 0 | 0 |
| 13 | 72 | 12 | 1 | 1 |
| 14 | 22 | 13 | 11 | 11 |
| 15 | 198 | 175 | 165 | 163 |
| Total: | 2,694 | 1,345 | 1,062 | 958 |

TABLE 10
Assessing clonal expansion among
post-infusion CAR T cells

| | clone | 1 < clone | clone |
|---|---|---|---|
| Patient | size = 1 | size <= 10 | size > 10 |
| 0 | N/A | N/A | N/A |
| 1 | N/A | N/A | N/A |
| 2 | 979 | 11 | 0 |
| 3 | 169 | 0 | 0 |
| 4 | 1,150 | 133 | 1 |
| 5 | 5,184 | 178 | 0 |
| 6 | 1,302 | 185 | 2 |
| 7 | 87 | 4 | 0 |
| 8 | 4,958 | 129 | 1 |
| 9 | 3,323 | 107 | 0 |
| 10 | 7,085 | 396 | 12 |
| 11 | 8,028 | 639 | 4 |
| 12 | 180 | 1 | 0 |
| 13 | 2,824 | 209 | 0 |
| 14 | 4,807 | 156 | 4 |
| 15 | 4,905 | 472 | 43 |
| Total: | 44,981 | 2,620 | 67 |

To determine the trajectories of particular post-infusion cell states, first the earliest time point that a specific TCR lineage was observed was identified, next identified the cluster encompassing that clone at that earliest time point, and then determined the cluster that housed the final time point where that TCR clonotype was observed (FIGS. 6B-C). The lineages across CD8+ clusters were the focus because very few lineages were identified within the CD4+ post-infusion cells, reflecting a relative underabundance of CD4+ CAT T cells observed post-infusion samples. Interestingly, multiple G αβTCRs mapped across the functional effector clusters 3 and 8, potentially suggesting that CD8+ functional effector subsets share common origins. For instance, CAR T cells within cluster 3 (GZMK+ effectors) at relatively early time points often shared TCRs with cells from cluster 8 (GZMB+ effectors) at later time points, again suggesting that GZMK+ expressing CD8+ T cells can be an earlier effector state immediately preceding cytotoxic effectors (Li 2019). However, evidence of the opposite phenomenon occurring were also observed, where GZMB-expressing cluster 8 lineages later acquire the GZMK+ effector signature (FIG. 6C). Furthermore, pseudotime analysis suggested that dysfunctional cell states, consisting primarily of clusters 13 and 20, arise from two transcriptional pathways: early dysfunction rapidly acquired after infusion, or late dysfunction acquired as a consequence of sustained effector function. Some post-infusion lineages in clusters 3 and 8 at early time points that were also seen in cluster 13 at later timepoints were observed, which may represent the conventional loss of effector potential and onset of exhaustion in the functional effectors, or late dysfunction. Some lineages from CD8+ CAR cell product, specifically from cluster 1, were mapped to cluster 13 upon infusion (FIG. 6D-E), suggesting early dysfunction. However, establishing conclusive evidence of early dysfunction using TCR lineages remains complicated by relatively limited sampling of extremely diverse populations.

TCR lineages were used to identify precursors within the CAR product that gave rise to the predominant functional effector populations observed throughout post-infusion time points, once again concentrating on CD8+ lineages due to the low frequency of CD4+ lineages in the dataset (FIG. 7B). Post-infusion clonotypes were identified that shared exact up TCRs with CAR product cells (FIG. 6E). Because the effector populations exhibit distinct transcriptional signatures, it was hypothesized that precursor lineages within the CAR product would fall within a limited set of clusters. However, these effector lineages linked to several CAR product clusters, including 1, 5, 7, and 12 (FIGS. 6E-6G). Therefore, based on global gene expression analysis alone, no single CAR product cluster contained the precursors for the potent effector lineages.

### Example 5. Effector CAR Product Precursors have an Identifiable Signature Pre-Infusion

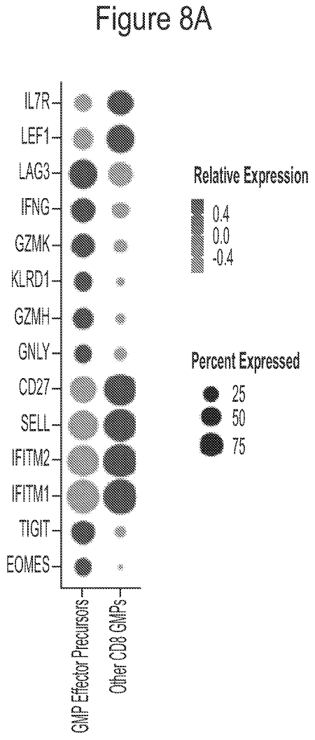

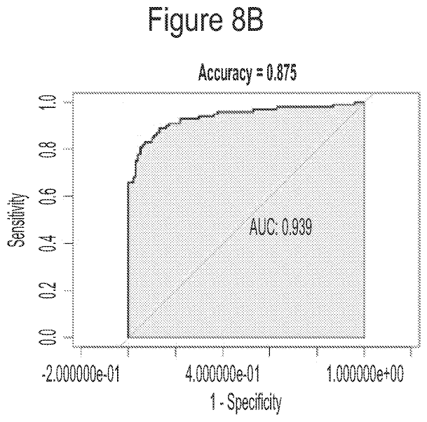

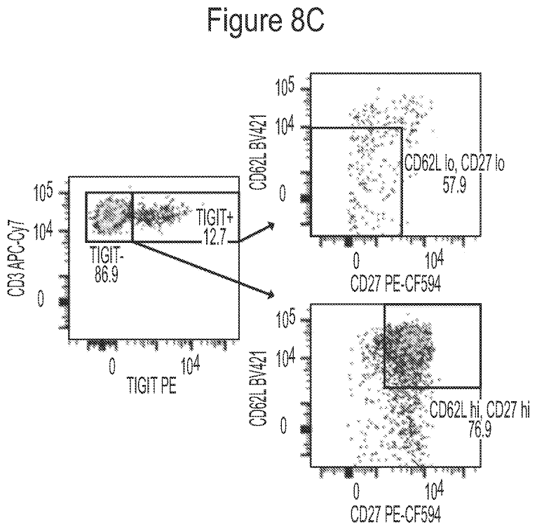

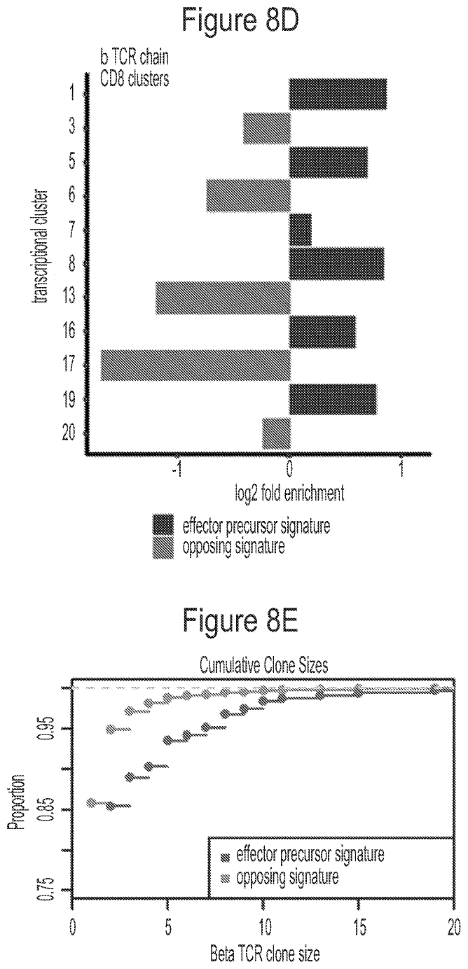

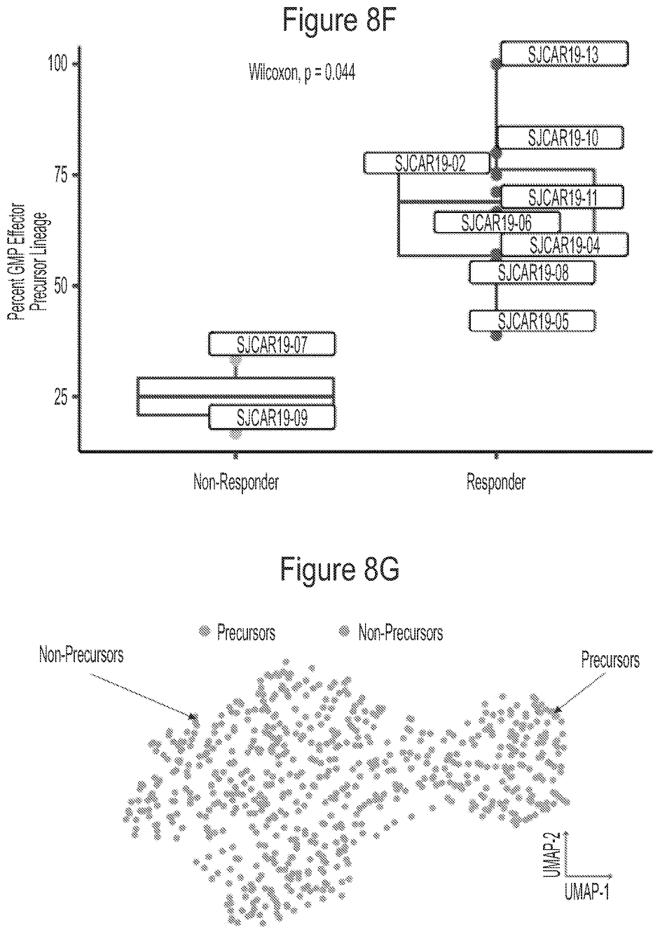

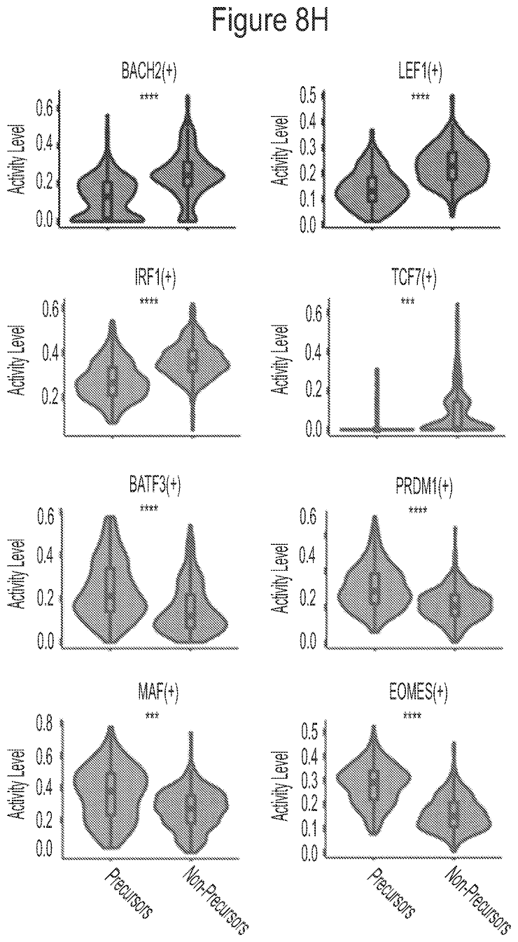

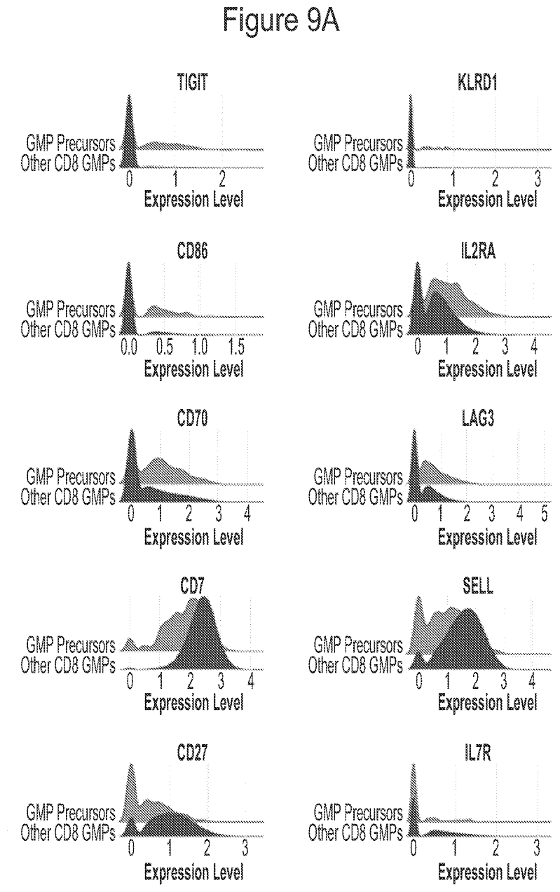

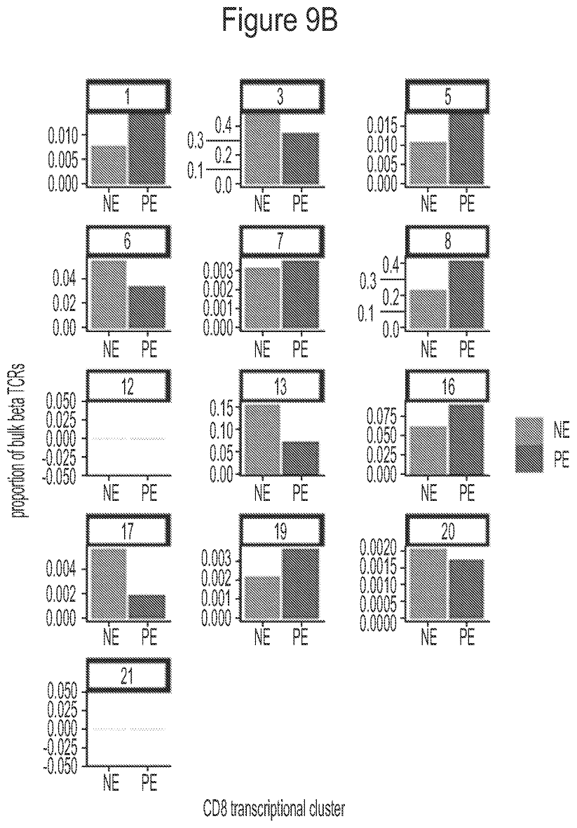

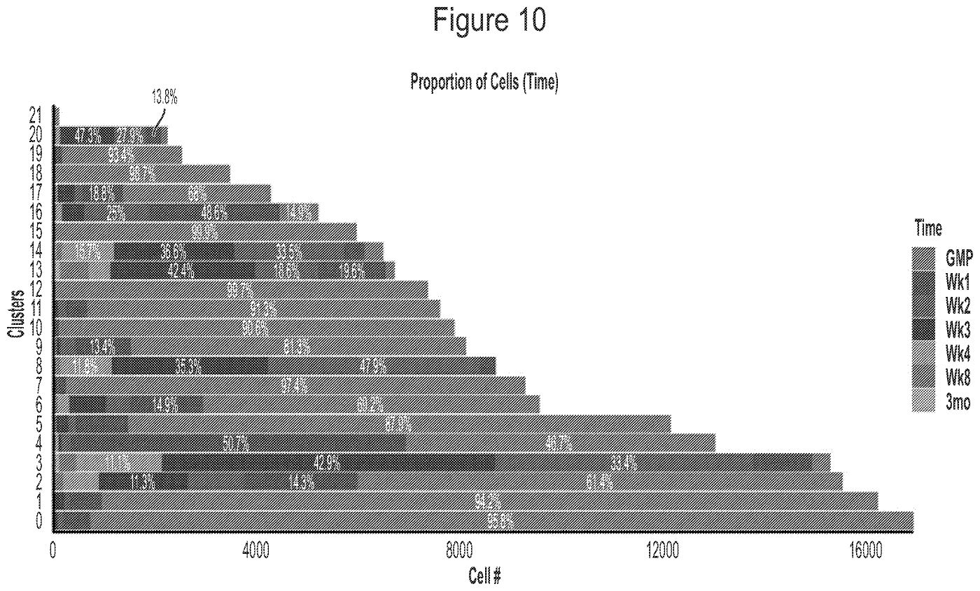

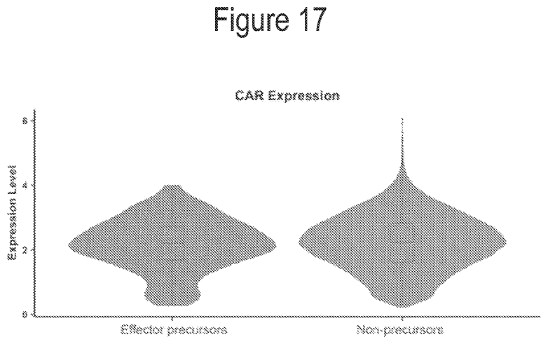

Since global gene expression analysis did not identify a single effector precursor transcriptional cluster within the CAR product, it was hypothesized that more subtle gene signatures that were obscured from broader analyses might characterize a pre-effector phenotype. To test this hypothesis, the transcriptional profiles of CAR product cells that shared TCRs were compared with cells found in post-infusion effector clusters 3 and 8 to all other CD8+ CAR T cells of the CAR product whose TCRs were never observed post-infusion (CAR product controls). This comparison identified both upregulated and downregulated genes associated with the precursors that share lineages with functional effector responses (FIG. 8A; FIG. 9A). Particularly, lineage-defined effector precursors were, prior to infusion, already expressing genes associated with an effector T cell phenotype, including classic effector markers EOMES, GNLY, GZMH, GZMK, and IFNG. Surprisingly, genes that are often associated with impaired T cell activation and effector function, such as TIGIT and LAG3, were also upregulated among the effector precursors when compared to the CAR product controls. Other downregulated genes within the effector precursors relative to control cells were associated with T cell memory subsets. For example, effector precursors significantly downregulated central memory genes SELL, which encodes for CD62L, and IL7R, as well as the stem cell memory gene LEF1. Although previous CD19-CAR T cell studies have identified CD27 as a marker for optimal CART cell performance (Fraitta and Chen), in the present analysis the CAR product clones that also appear in post-infusion effector clusters exhibited markedly lower levels of CD27 gene expression in the product. Notably, no difference in CAR expression was found between lineage-defined effector precursors and other CD8+ CAR T cells in the GMP product (FIG. 17; adjusted p-value=1). However, the percentage of lineages spanning the GMP product and post-infusion samples that anchored specifically in cytotoxic effector clusters 3 and 8 was significantly lower in non-responders versus responders to treatment, although this analysis was notably limited by the small number of non-responders with available lineage data (n=2) and the restricted number of lineages available for some patients (FIG. 8F; Table 9).

To investigate potential mechanisms into the processes distinguishing lineage-defined effector precursors from other GMP cells, the activity of transcriptional regulatory networks was inferred using SCENIC on a subset of the GMP CD8+ CAR T cells, specifically including all cells with lineages tracing to post-infusion effector clusters 3 and 8. Despite some overlap, most precursor effectors clustered distinctly from other cells in the SCENIC analysis, suggesting that defined regulatory networks underlie the expression differences between these and other pre-infusion cells (FIG. 8G). In particular, effector precursors showed significant upregulation of regulons of known T cell survival and effector differentiation genes, including regulons for BATF3(+), PRDMI(+), MAF(+), and EOMES(+), and significant downregulation of stem-cell memory regulons LEF1(+) and TCF7(+), among others, suggesting that effector precursor CAR T cells are primed to differentiate into potent cytotoxic effectors rather than multipotent memory T cells with enhanced self-renewal capacity (FIG. 8H). It is important to emphasize that this analysis serves as an independent, orthogonal validation of the pre-effector signature defined using transcriptional comparisons distinguishing effector precursors. Here, the SCENIC analysis was applied to define the variation in transcriptional regulons across the GMP product, and it independently identified the effector precursors as clustering separately based on inferred transcription factor activity.

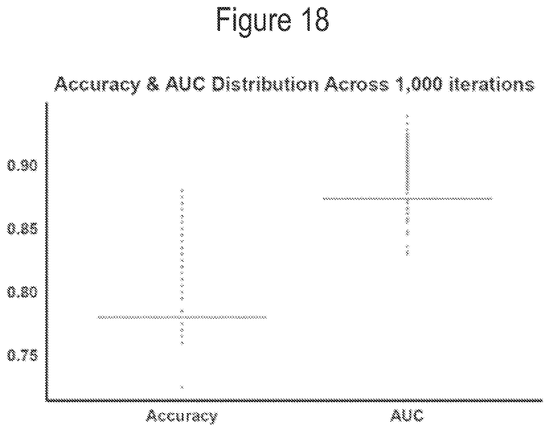

As a profile unique to pre-effector CAR product cells was able to be found, it was next tested whether this signature could be used to train a classifier that could assess CAR T cell effector potential in other CAR products. The top 100 differentially expressed genes between downsampled pre-effector and other downsampled CD8+ CAR product cells were selected to train a machine learning algorithm to predict whether a given CAR T cell product would give rise to a lineage that was observed in the CD8 effector pool after infusion. LOOCV (leave-one-out cross validation) was used to validate the accuracy of the model, and the differentially expressed signatures were also computed only from each training set. Due to the immense size of the dataset, the data were downsampled for the classification analyses. Using 1,000 randomly downsampled sets of CAR T cells, the best performance of the classifier (run over 1000 times on different downsampled sets of cells) reported an accuracy of 87.5% and an AUC of 0.939, with an overall average of 78% accuracy and an AUC of 0.876 (FIG. 8B) across the 1,000 iterations (FIGS. 8B and 18).

It was theorized that if the preparation of CAR T cell product cell precursor signature indeed correlates with the eventual development of functional effector CAR T cells, selectively isolating these precursor cells should enrich pre-effector associated TCR clonotypes. To test this, a subset of genes was selected that encode for surface proteins that were sufficiently differentially expressed between pre-effector CAR T cells and all other CAR T cells from the preparation of CAR T cells (FIG. 9A): TIGIT, SELL (CD62L), and CD27. CD8+ CAR T cells that contained the predicted effector precursor profile (TIGIT+, CD62Llo and CD27neg) were sorted from a cryopreserved preparation of CAR T cells from a single patient. TIGIT+ cells represented 12.7% of CD8+ CAR T cells, and a further subset of cells that were low for both CD62L and CD27 (57.9% of TIGIT+ cells) were identified. CD62L and CD27 mid-hi expressing TIGIT-cells were also sorted (74.9% of TIGIT-cells) to use as a population representing an opposing, non-effector precursor phenotype (FIG. 8C). The complementarity determining region 3 (CDR3) of the β TCR chain were next sequenced within the bulk RNA obtained from each sorted population to compare the relative abundance of effector precursor lineages in each sorting scheme. Because of dramatic differences in sample sizes and the extreme diversity of the repertoires, the 1,000 most frequently observed R TCRs from each sort condition were first compared to those observed in the single-cell post infusion data; interestingly, the top clones from the effector precursor sort were more than twice as likely to be observed post infusion than the top clones from the non-effector sort (effector: 132; non-effector: 59; Fisher's exact test, p<0.001). When considering all bulk-sorted TCRβ nucleotide sequences that matched those in the patient's post infusion CD8 repertoire, marked differences in the proportion of the inferred transcriptional clusters to which each sort signature mapped were observed (FIG. 9B). The precursor effector signature-sorted cells expressed TCRs that were more likely to be observed in proliferating CD8 clusters (1, 5, 7, 19), early CD8 effector cluster 16, and GZMB CD8 effector cluster 8 (FIG. 8D), when compared to the opposing signature. In contrast, the opposing, non-effector phenotype was primarily enriched for transitioning CD8 clusters (6 and 17) and functionally exhausted CD8 cluster 13, with a slight enrichment for GZMK cluster 3. Post-infusion cells sharing TCRs from the precursor effector sorting scheme were also significantly more clonally expanded (Kolmogorov-Smirnov test, p<0.001; FIG. 8D). These analyses confirmed that the transcriptional profile associated with precursors of effector lineages was also reflected on the cell surface and demonstrated that the use of surface markers identified by the analyses could be utilized to enrich preparation of CAR T cells primed to become potent cytotoxic effectors.

After confirming that sorting for this pre-effector signature can enrich for cells with effector preparation of CAR T cell product precursor-associated TCR lineages, whether the pre-effector signature was associated with CAR T cell expansion was assessed, as judged by qPCR, post infusion. To determine how a patient's preparation of CAR T cell product aligns with the identified pre-effector signature, a gene set module score was utilized to identify cells in the preparation of CAR T cell product with expression profiles most comparable to the TCR-defined precursor lineages.

The present study characterized and tracked the significant heterogeneity in pre-infusion CAR T cells. Upon infusion into the patient, different CAR T cell subsets lead to divergent differentiation trajectories. The first trajectory involved functional effector differentiation characterized by expression of conventional cytotoxic genes, such as the granzymes, PRF1, and NKG7, among others. As expected, this trajectory eventually culminated in T cell exhaustion, with the expression of TOX and other inhibitory proteins, and cell death as evidenced by an upregulation in CASP8. Alternatively, another trajectory indicated rapid development of these same exhaustion and cell death signatures soon after infusion. By using the endogenous TCR as a method to track CAR T cell lineages, it was discovered herein that the CAR product cells giving rise to optimal effector phenotypes corresponded to a unique subset with a statistically robust transcriptional signature; the presence of this signature in CAR T cells of the CAR product consequently impacts functional effector differentiation and proliferative capacity post-infusion.

It was surprising to observe TIGIT upregulation in the CAR product precursors of the functional effector population, as this gene is known as a negative regulator of T cell function. TIGIT functions primarily to inhibit T cell effector function by competing with CD266 for binding to CD155 on dendritic cells (Yu 2009). To add to its inhibitory effect, TIGIT also prevents homodimerization of CD266 to directly restrain costimulatory signaling (Johnston 2014). The CAR T cells that express TIGIT highly may actually benefit from its inhibitory effects by limiting the strength of early effector responses that could ultimately lead to a premature dysfunctional state. Under this assumption, overstimulated CAR T cells without TIGIT would become overactivated and, subsequently, exhaust themselves early in the treatment window. These data demonstrate the potential utility in harnessing the role of inhibitory molecules to increase the magnitude and persistence of effector responses.

The present data could have significant implications for anti-CD19 CAR-based therapies. The classifier generated from the gene expression differences between pre-effector CAR and the other CAR product cells (FIG. 8B) can be used early in treatment, after creating the CAR product, to determine how efficacious CAR T cell therapy may be once infused and whether another treatment protocol should be employed instead. Moreover, the present findings seem to suggest that, out of the entire population of the CAR product, only a small proportion of CAR T cells go on to do the actual work of tumor killing. Using the genes identified in the pre-effector signature provided herein, a suite of surface markers could potentially be used to enrich the most ideal CAR T cells within the CAR product. This would allow physicians to infuse patients with a higher percentage of the pre-effector population and with fewer of the CAR T cells that take on a dysfunctional phenotype upon infusion. The present disclosure provides an innovative method to identify gene signatures in CAR types by tracking endogenous TCR sequences. Overall, the present data provide unique opportunities to fine-tune CAR T cell treatment protocols to normalize the variability of patient outcomes following CAR T cell therapy.

TABLE 11
CD19.4-1BB.zeta CAR-amino acid sequence

| SEQ ID | | |
|---|---|---|
| NO | Sequence Name | Sequence |
| 1 | CD8a Leader | MALPVTALLLPLALLLHAARPD |
| 2 | FMC63scFv-VL | IQMTQTTSSLSASLGDRVTISCRASQDISKYLNWYQQKPDGT |
| | | VKLLIYHTSRLHSGVPSRFSGSGSGTDYSLTISNLEQEDIAT |
| | | YFCQQGNTLPYTFGGGTKLEIT |
| 3 | Linker | GGGGSGGGGSGGGGS |
| 4 | FMC63scFv-VH | EVKLQESGPGLVAPSQSLSVTCTVSGVSLPDYGVSWIRQPPR |
| | | KGLEWLGVIWGSETTYYNSALKSRLTIIKDNSKSQVELKMNS |
| | | LQTDDTAIYYCAKHYYYGGSYAMDYWGQGTSVTVSS |
| 5 | CD8a | TTTPAPRPPTPAPTIASOPLSLRPEACRPAAGGAVHTRGLDF |
| | Hinge/ | ACDIYIWAPLAGTCGVLLLSLVITLYC |
| | transmembrane | |
| | domain | |
| 6 | 4-1BB costim | KRGRKKLLYIFKQPFMRPVQTTQEEDGCSCRFPEEEEGGCEL |
| | domain | |
| 7 | CD3zeta | RVKFSRSADAPAYQQGONQLYNELNLGRREEYDVLDKRRGRD |
| | | PEMGGKPRRKNPQEGLYNELQKDKMAEAYSEIGMKGERRRGK |
| | | GHDGLYQGLSTATKDTYDALHMQALPPR |
| 8 | CD8a Leader | ATGGCCTTACCAGTGACCGCCTTGCTCCTGCCGCTGGCCTTG |
| | | CTGCTCCACGCCGCCAGGCCG |
| 9 | FMC63scFv-VL | GACATCCAGATGACACAGACTACATCCTCCCTGTCTGCCTCT |
| | | CTGGGAGACAGAGTCACCATCAGTTGCAGGGCAAGTCAGGAC |
| | | ATTAGTAAATATTTAAATTGGTATCAGCAGAAACCAGATGGA |
| | | ACTGTTAAACTCCTGATCTACCATACATCAAGATTACACTCA |
| | | GGAGTCCCATCAAGGTTCAGTGGCAGTGGGTCTGGAACAGAT |
| | | TATTCTCTCACCATTAGCAACCTGGAGCAAGAAGATATTGCC |
| | | ACTTACTTTTGCCAACAGGGTAATACGCTTCCGTACACGTTC |
| | | GGAGGGGGGACCAAGCTGGAGATCACAGGT |
| 10 | Linker | GGCGGTGGCTCGGGCGGTGGTGGGTCGGGTGGCGGCGGATCT |
| 11 | FMC63scFv-VH | GAGGTGAAACTGCAGGAGTCAGGACCTGGCCTGGTGGCGCCC |
| | | TCACAGAGCCTGTCCGTCACATGCACTGTCTCAGGGGTCTCA |
| | | TTACCCGACTATGGTGTAAGCTGGATTCGCCAGCCTCCACGA |
| | | AAGGGTCTGGAGTGGCTGGGAGTAATATGGGGTAGTGAAACC |
| | | ACATACTATAATTCAGCTCTCAAATCCAGACTGACCATCATC |
| | | AAGGACAACTCCAAGAGCCAAGTTTTCTTAAAAATGAACAGT |
| | | CTGCAAACTGATGACACAGCCATTTACTACTGTGCCAAACAT |
| | | TATTACTACGGTGGTAGCTATGCTATGGACTACTGGGGCCAA |
| | | GGAACCTCAGTCACCGTCTCCTCA |
| 12 | CD8a Hinge/ | ACCACGACGCCAGCGCCGCGACCACCAACACCGGCGCCCACC |
| | transmembrane | ATCGCGTCGCAGCCCCTGTCCCTGCGCCCAGAGGCGTGCCGG |
| | domain | CCAGCGGCGGGGGGCGCAGTGCACACGAGGGGGCTGGACTTC |
| | | GCCTGTGATATCTACATCTGGGCGCCCTTGGCCGGGACTTGT |
| | | GGGGTCCTTCTCCTGTCACTGGTTATCACCCTTTACTGC |
| 13 | 4-1BB costim | AAACGGGGCAGAAAGAAACTCCTGTATATATTCAAACAACCA |
| | domain | TTTATGAGACCAGTACAAACTACTCAAGAGGAAGATGGCTGT |
| | | AGCTGCCGATTTCCAGAAGAAGAAGAAGGAGGATGTGAACTG |
| | | AGAGTG |
| 14 | CD3zeta | AAGTTCAGCAGGAGCGCAGACGCCCCCGCGTACCAGCAGGGC |
| | | CAGAACCAGCTCTATAACGAGCTCAATCTAGGACGAAGAGAG |
| | | GAGTACGATGTTTTGGACAAGAGACGTGGCCGGGACCCTGAG |
| | | ATGGGGGGAAAGCCGAGAAGGAAGAACCCTCAGGAAGGCCTG |
| | | TACAATGAACTGCAGAAAGATAAGATGGCGGAGGCCTACAGT |
| | | GAGATTGGGATGAAAGGCGAGCGCCGGAGGGGCAAGGGGCAC |
| | | GATGGCCTTTACCAGGGTCTCAGTACAGCCACCAAGGACACC |
| | | TACGACGCCCTTCACATGCAGGCCCTGCCCCCTCGC |

### Example 6. Immunophenotyping Confirms Expression and Validates Functional Differences

The present in-depth single cell expression analyses demonstrated clear transcriptional differences that were consistent across multiple patients and time points, transcriptional abundance does not necessarily correlate with protein abundance. Therefore, CAR T cells were immunophenotyped and, when sufficient sample was available, tested for functional activation via intracellular staining in the context of ex vivo coculture with CD19+ tumor cells.

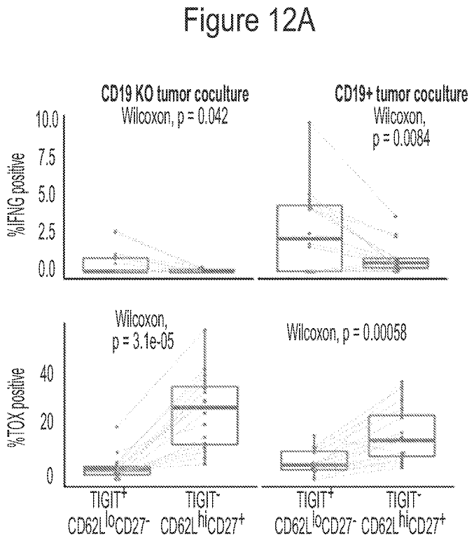

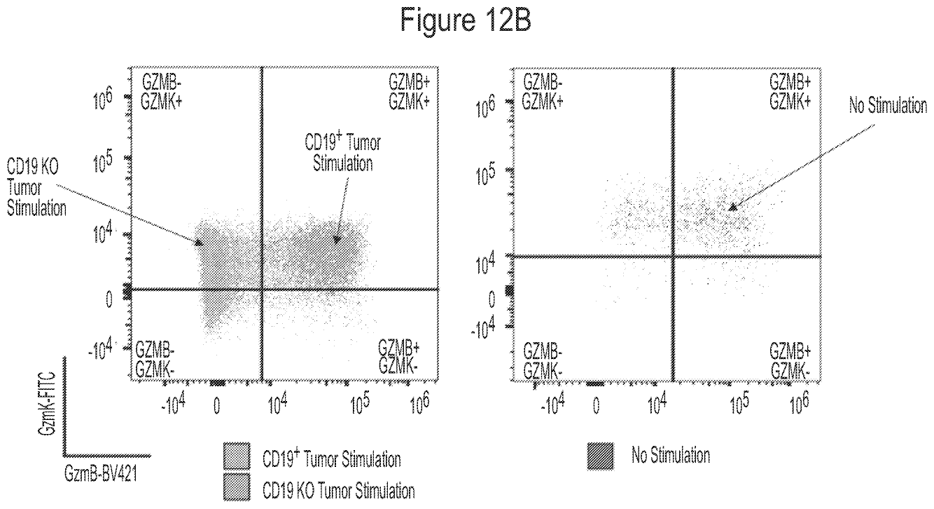

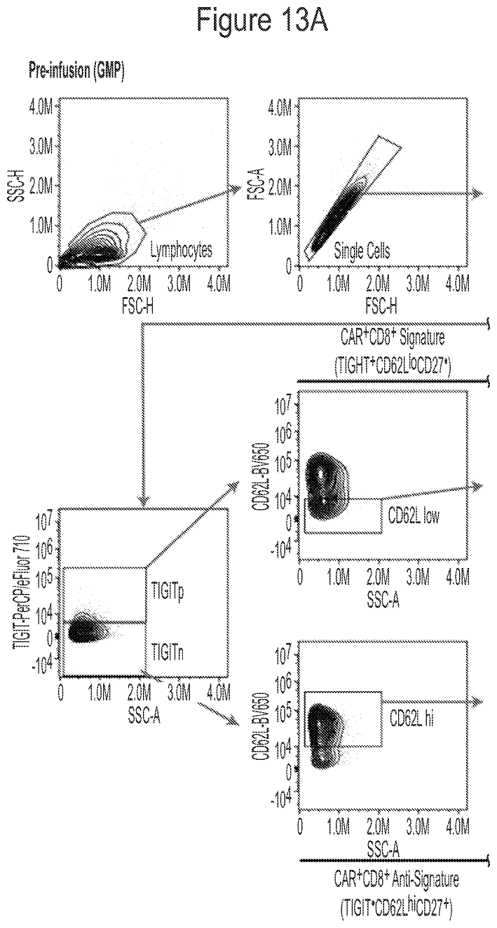

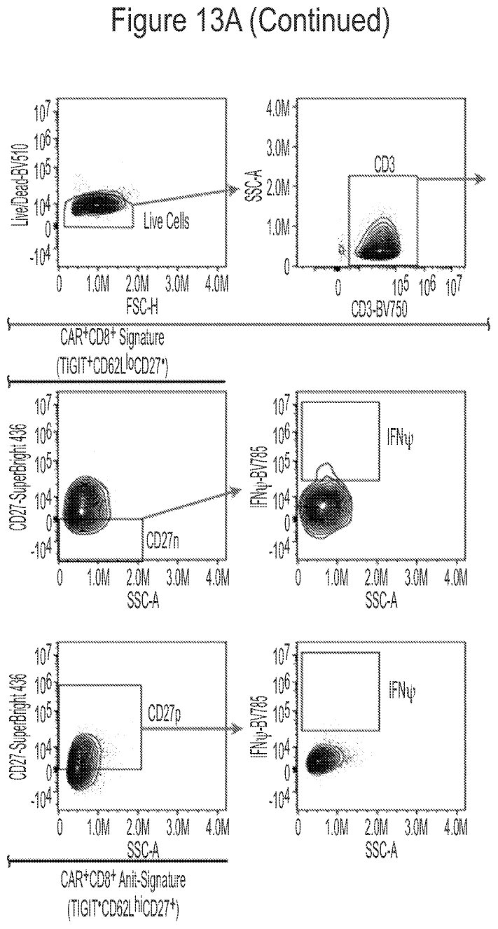

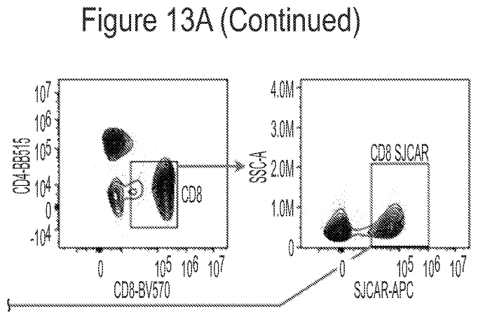

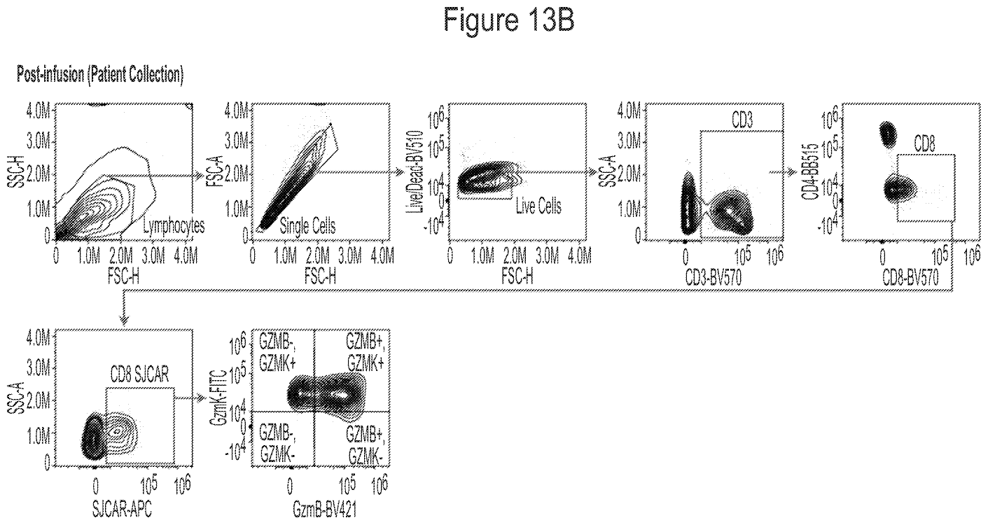

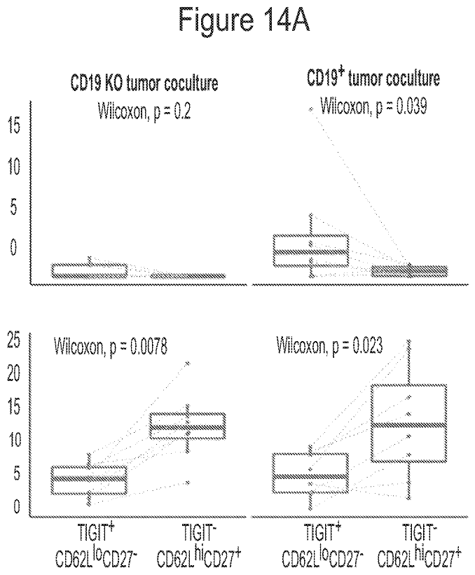

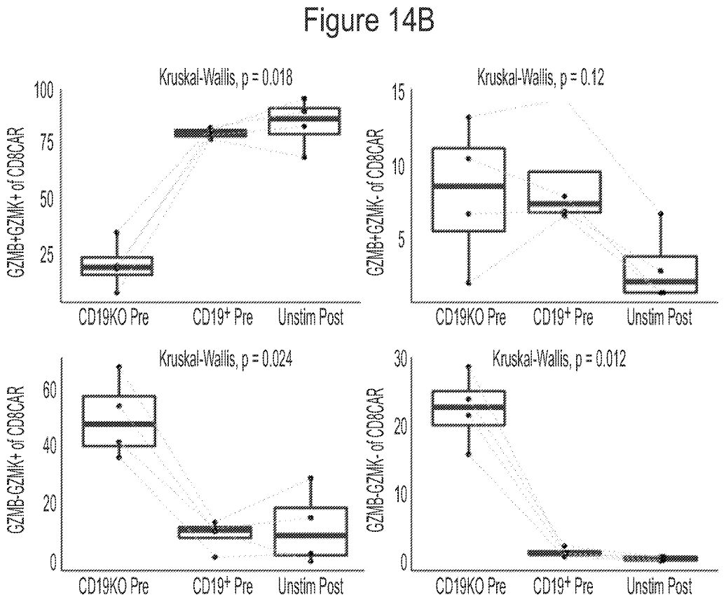

To functionally validate the effector precursor signature, GMP CAR T cells from patients 0-15 were co-cultured with either CD19+ tumor cells or tumor cells lacking the CD19 antigen and then assayed a panel of surface and intracellular proteins by flow cytometry. Notably, CD8+ CAR T cells with the precursor effector surface phenotype (TIGIT+CD27-CD62L^{lo}) were significantly more likely to produce interferon gamma (IFNγ) than cells with the opposite surface phenotype (TIGIT-CD27+CD62L^{hi}; FIG. 12A, top; FIG. 13A-13B). Concordantly, these effector precursor cells were also significantly less likely to produce TOX, the transcription factor associated with T cell exhaustion (FIG. 12A, bottom). These experiments were repeated with GMP CAR T cell samples from a distinct set of patients whose cells were not included in the initial transcriptional profiling dataset (patients 16-23) to independently validate the immediate functional superiority of the effector precursor cells in the context of CD19+ tumor stimulation. The results replicated those from the original cohort (FIG. 14A), indicating that pre-infusion CAR T cells of the GMP product with the precursor effector surface phenotype rapidly produce IFNγ upon stimulation with antigen. Critically, cells within the opposing phenotype develop a dysfunctional profile immediately after stimulation. CD8+ CAR T cells with the effector precursor phenotype constituted a small proportion of the total CD8+ CAR+T GMP cells, representing a mean percent of 1.4% across all subjects, while the opposing surface phenotype was observed in 32.9% of CD8+ CAR T cells.

Lastly, it was sought to confirm at the protein level the development of the phenotypes associated with the transcriptionally defined cytotoxic effector clusters 3 and 8. Among the pre-infusion CD8+ CAR T cells, distinct populations on the basis of GZMB and GZMK staining were observed both before and after stimulation with the CD19+ tumor cell line. Specifically, GZMK+GZMB+ cells made up an average of 17.6% percent of CD8+ CAR T cells prior to stimulation but increased significantly upon stimulation to an average of 64.3%, at which point the double-positive population predominated in the cultures (FIG. 12B left; FIG. 14B). This increase in GZMK+GZMB+ cells was due to decreases in the proportions of GZMK−GZMB− and GZMK+GZMB− cells, as GZMK−GZMB+ cell proportions did not change upon stimulation (FIG. 14B). These population proportions broadly match those observed in single-cell expression analyses, where the transcriptionally defined GZMK+GZMB+ cluster 3 made up the vast majority of cytotoxic CD8+ T cells (FIGS. 1A and 1B). Post-infusion CD8+ CAR T cells from the sample obtained at peak expansion displayed the same pattern of GZMB and GZMK production as pre-infusion CD8+ CAR T cells having undergone CD19+ stimulation, confirming that CAR T cells directly isolated from the patient were activated (FIG. 12B, right).

### REFERENCES

1. Park J H, Rivière I, Gonen M, Wang X, Sénéchal B, Curran K J, et al. Long-Term Follow-up of CD19 CAR Therapy in Acute Lymphoblastic Leukemia. N Engl J Med. 2018; 378:449-59.
2. Davila M L, Riviere I, Wang X, Bartido S, Park J, Curran K, et al. Efficacy and toxicity management of 19-28z CAR T cell therapy in B cell acute lymphoblastic leukemia. Sci Transl Med. 2014; 6:224ra25.
3. Maude S L, Frey N, Shaw P A, Aplenc R, Barrett D M, Bunin N J, et al. Chimeric antigen receptor T cells for sustained remissions in leukemia. N Engl J Med. 2014; 371:1507-17.
4. Lee D W, Kochenderfer I N, Stetler-Stevenson M, Cui Y K, Delbrook C, Feldman S A, et al. T cells expressing CD19 chimeric antigen receptors for acute lymphoblastic leukaemia in children and young adults: a phase 1 dose-escalation trial. Lancet Lond Engl. 2015; 385:517-28.
5. Finney O C, Brakke H M, Rawlings-Rhea S, Hicks R, Doolittle D, Lopez M, et al. CD19 CAR T cell product and disease attributes predict leukemia remission durability. J Clin Invest. 2019; 129:2123-32.
6. Chen G M, Chen C, Das R K, Gao P, Chen C-H, Bandyopadhyay S, et al. Integrative Bulk and Single-Cell Profiling of Premanufacture T-cell Populations Reveals Factors Mediating Long-Term Persistence of CAR T-cell Therapy. Cancer Discov. American Association for Cancer Research; 2021; 11:2186-99.
7. Fraietta J A, Lacey S F, Orlando E J, Pruteanu-Malinici I, Gohil M, Lundh S, et al. Determinants of response and resistance to CD19 chimeric antigen receptor (CAR) T cell therapy of chronic lymphocytic leukemia. Nat Med. 2018; 24:563-71.
8. Sheih A, Voillet V, Hanafi L-A, DeBerg H A, Yajima M, Hawkins R, et al. Clonal kinetics and single-cell transcriptional profiling of CAR-T cells in patients undergoing CD19 CAR-T immunotherapy. Nat Commun. 2020; 11:219.
9. Chan W K, Suwannasaen D, Throm R E, Li Y, Eldridge P W, Houston J, et al. Chimeric antigen receptor-redirected CD45RA-negative T cells have potent antileukemia and pathogen memory response without graft-versus-host activity. Leukemia. 2015; 29:387-95.
10. Imai C, Mihara K, Andreansky M, Nicholson I C, Pui C-H, Geiger T L, et al. Chimeric receptors with 4-1BB signaling capacity provoke potent cytotoxicity against acute lymphoblastic leukemia. Leukemia. 2004; 18:676-84.
11. Bratke K, Kuepper M, Bade B, Virchow J C, Luttmann W. Differential expression of human granzymes A, B, and K in natural killer cells and during CD8+ T cell differentiation in peripheral blood. Eur J Immunol. 2005; 35:2608-16.
12. Wherry E J. T cell exhaustion. Nat Immunol. 2011; 12:492-9.
13. Galletti G, De Simone G, Mazza E M C, Puccio S, Mezzanotte C, Bi T M, et al. Two subsets of stem-like CD8+ memory T cell progenitors with distinct fate commitments in humans. Nat Immunol. 2020; 21:1552-62.
14. de Greef P C, Oakes T, Gerritsen B, Ismail M, Heather J M, Hermsen R, et al. The naive T-cell receptor repertoire has an extremely broad distribution of clone sizes. Walczak A M, Rath S, Pogorelyy M V, editors. eLife. eLife Sciences Publications, Ltd; 2020; 9:e49900.
15. Laydon D J, Bangham C R M, Asquith B. Estimating T-cell repertoire diversity: limitations of classical estimators and a new approach. Philos Trans R Soc B Biol Sci. 2015; 370:20140291.
16. Qi Q, Liu Y, Cheng Y, Glanville J, Zhang D, Lee J-Y, et al. Diversity and clonal selection in the human T-cell repertoire. Proc Natl Acad Sci. National Academy of Sciences; 2014; 111:13139-44.
17. Li H, Leun A M van der, Yofe I, Lubling Y, Gelbard-Solodkin D, Akkooi A C J van, et al. Dysfunctional CD8 T Cells Form a Proliferative, Dynamically Regulated Compartment within Human Melanoma. Cell. Elsevier; 2019; 176:775-789.e18.
18. Li X, Guo X, Zhu Y, Wei G, Zhang Y, Li X, et al. Single-Cell Transcriptomic Analysis Reveals BCMA CAR-T Cell Dynamics in a Patient with Refractory Primary Plasma Cell Leukemia. Mol Ther. 2021; 29:645-57.
19. Yu X, Harden K, C Gonzalez L, Francesco M, Chiang E, Irving B, et al. The surface protein TIGIT suppresses T cell activation by promoting the generation of mature immunoregulatory dendritic cells. Nat Immunol. 2009; 10:48-57.
20. Johnston R J, Comps-Agrar L, Hackney J, Yu X, Huseni M, Yang Y, et al. The immunoreceptor TIGIT regulates antitumor and antiviral CD8(+) T cell effector function. Cancer Cell. 2014; 26:923-37.
21. Eyquem J, Mansilla-Soto J, Giavridis T, van der Stegen S J C, Hamieh M, Cunanan K M, et al. Targeting a CAR to the TRAC locus with CRISPR/Cas9 enhances tumour rejection. Nature. 2017; 543:113-7.
22. Boroughs A C, Larson R C, Marjanovic N D, Gosik K, Castano A P, Porter C B M, et al. A Distinct Transcriptional Program in Human CAR T Cells Bearing the 4-1B B Signaling Domain Revealed by scRNA-Seq. Mol Ther J Am Soc Gene Ther. 2020; 28:2577-92.
23. Hao Y, Hao S, Andersen-Nissen E, Mauck W M, Zheng S, Butler A, et al. Integrated analysis of multimodal single-cell data. Cell. 2021; 184:3573-3587.e29.
24. Cores C, Vapnik V N. Support-vector networks. Machine Learning, 1995; 20(3):273-297.
25. Qiu X, Hill A, Packer J, Lin D, Ma Y, Trapnell C. Single-ceH nRN A quantification and differential analysis with Census. Nature Methods. 2017; 14:309-315.
26. Talleur A C, Qudeinat A, Metais J Y, Langfitt D, Mamcarz E, Crawford J C. Preferential expansion of CD8+ CD19-CAR T cells post infusion and role of disease burden on outcome in pediatric B-ALL. In review

All references cited anywhere within this specification are incorporated herein by reference in their entirety for all purposes.

While preferred embodiments of the present invention have been shown and described herein, it will be obvious to those skilled in the art that such embodiments are provided by way of example only.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-107. (canceled)

108. A method of determining the cytotoxic effector potential of a preparation of CAR T cells, the method comprising:
a) acquiring single-cell gene expression data of the preparation of CAR T cells for a set of differentially expressed markers which are expressed at different levels in CAR T cells that differentiate into cytotoxic effector CAR T cells upon administration to a subject, and
b) determining the cytotoxic effector potential indicating the likelihood of the CAR T cell preparation, when administered, to give rise to post-infusion cytotoxic effector CAR T cells.

109. The method of claim 108, wherein the set of differentially expressed markers are expressed at different levels in CAR T cells of the preparation that are clonally related to post-infusion CAR T cells that have cytotoxic effector function.

110. The method of claim 108, wherein the cytotoxic effector potential of the preparation of CAR T cells is determined by training a machine learning classifier with a training set of the differentially expressed markers to generate a value reflecting the probability that an individual CAR T cell of the preparation of CAR T cells is a cytotoxic effector precursor.

111. The method of claim 108, wherein the differently expressed markers comprises the genes of Table 1, Table 2, Table 3, Table 4, FIG. 1B, genes of clusters 1, 3, 5, 7, 8, 12 and/or 14 of FIG. 1B and/or 3, 8, and/or 14 of FIG. 1B.

112. The method of claim 108, wherein the differently expressed markers comprises the top 1, 2, 3, 4, 5, 10, 15, 20, 25, 30, 35, 40, 45, 50, 55, 60, 65, 70, 75, 80, 85, 90, 95, or 100 genes (or integers in between) of a pre-effector gene signature generated by a method comprising the steps of:
i) measuring single-cell gene expression data and endogenous T cell receptor (TCR) sequencing data of the preparation of CAR T cells;
ii) administering the preparation of CAR T cells to a subject;
iii) isolating post-infusion CAR T cells at one or more time points after the administration step ii);
iv) identifying cytotoxic effector CAR T cells within the post-infusion CAR T cells isolated in step iii);
v) acquiring single-cell gene expression data and endogenous TCR sequencing data of the cytotoxic effector CAR T cells identified in step iv);
vi) using the endogenous TCR sequencing data obtained in steps i) and v) to determine which CAR T cells from the preparation of CAR T cells are cytotoxic effector precursors by determining which of the CAR T cells of the preparation of CAR T cells share a clonally related TCR with the post-infusion cytotoxic effector CAR T cells; and
vii) generating a pre-effector gene signature by determining which set of genes are differentially expressed in the cytotoxic effector precursors as compared to other or all other CAR T cells from the preparation.

113. The method of claim 108, wherein the cytotoxic effector potential of the preparation of CAR T cells or a subset of CAR T cells therefrom is determined by using flow cytometry to identify the CAR T cells of the preparation of CAR T cells that express a gene product of the set of differentially expressed markers.

114. The method of claim 108, wherein the cytotoxic effector potential of the preparation of CAR T cells or a subset of CAR T cells therefrom is determined by measuring protein expression of a gene product of the set of differentially expressed markers.

115. The method of claim 108, wherein the cytotoxic effector potential of the preparation of CAR T cells or a subset of CAR T cells therefrom is determined by measuring gene expression of the set of differentially expressed markers.

116. The method of claim 108, wherein the differently expressed markers comprises a pre-effector gene signature generated by a method comprising the steps of:
i) measuring single-cell gene expression data and endogenous T cell receptor (TCR) sequencing data of the preparation of CAR T cells;
ii) administering the preparation of CAR T cells to a subject;
iii) isolating post-infusion CAR T cells at one or more time points after the administration step ii);
iv) identifying cytotoxic effector CAR T cells within the post-infusion CAR T cells isolated in step iii);
v) acquiring single-cell gene expression data and endogenous TCR sequencing data of the cytotoxic effector CAR T cells identified in step iv);
vi) using the endogenous TCR sequencing data obtained in steps i) and v) to determine which CAR T cells from the preparation of CAR T cells are cytotoxic effector precursors by determining which of the CAR T cells of the preparation of CAR T cells share a clonally related TCR with the post-infusion cytotoxic effector CAR T cells; and
vii) generating a pre-effector gene signature by determining which set of genes are differentially expressed in the cytotoxic effector precursors as compared to other or all other CAR T cells from the preparation, and
wherein the cytotoxic effector potential of the preparation of CAR T cells or a subset of CAR T cells therefrom is determined by comparing the single-cell gene expression data of the preparation of CAR T cells or the subset of CAR T cells therefrom with the pre-effector gene signature and determining the preparation of CAR T cells or a subset of CAR T cells therefrom has cytotoxic effector potential if the preparation of CAR T cells or a subset of CAR T cells therefrom shares at least one or more marker genes from the pre-effector gene signature.

117. The method of claim 108, wherein the set of differentially expressed markers comprises one or more marker genes selected from LTB, EOMES, GNLY, GZMA, GZMH, GZMK, KLRD1, IFNG, CD52, CD74, CD86, LAG3, CASP8, TOX, TIGIT, CD27, SELL, CD44, CD70, HLA-DQA1, HLA-DR, HLA-DQ, PRF1, CDC20, PLK1, MKI67, and IL-7R.

118. The method of claim 108, wherein the set of differentially expressed markers comprises increased expression of TIGIT and/or LAG3 genes.

119. The method of claim 108, wherein the set of differentially expressed markers comprises decreased expression of CD27 gene.

120. The method of claim 108, wherein the set of differentially expressed markers are selected from CD62L, TIGIT, and CD27 genes.

121. The method of claim 108, wherein pre-effector gene signature comprises expression levels of genes that correspond to cell-surface expressed proteins.

122. A preparation of CAR T cells with cytotoxic effector potential determined by the method of claim 108.

123. A method for generating a pre-effector gene signature for determining the cytotoxic effector potential of a preparation of chimeric antigen receptor (CAR) T cells, the method comprising:
a) measuring single-cell gene expression data and endogenous T cell receptor (TCR) sequencing data of the preparation of CAR T cells;
b) administering the preparation of CAR T cells to a subject;
c) isolating post-infusion CAR T cells at one or more time points after the administration step b);
d) identifying cytotoxic effector CAR T cells within the post-infusion CAR T cells isolated in step c);
e) acquiring single-cell gene expression data and endogenous TCR sequencing data of the cytotoxic effector CAR T cells identified in step d);
f) using the endogenous TCR sequencing data obtained in steps a) and e) to determine which CAR T cells from the preparation of CAR T cells are cytotoxic effector precursors by determining which of the CAR T cells of the preparation of CAR T cells share a clonally related TCR with the post-infusion cytotoxic effector CAR T cells; and
g) generating a pre-effector gene signature by determining which set of genes are differentially expressed in the cytotoxic effector precursors as compared to other or all other CAR T cells from the preparation.

124. The method of claim 123, wherein step d) further comprises:
clustering single cell gene expression data of the post-infusion CAR T cell isolated in step c) using a clustering algorithm, resulting in a plurality of clusters such that each cluster in the plurality of clusters comprises similar single cell gene expression data within the cluster and such that single cell gene expression data differs across differing clusters; and
determining one or more functional effector cluster(s) of the plurality of clusters such that the one or more functional effector cluster(s) are associated with a functional effector T cell functional state; and
identifying the single cell gene expression data of cytotoxic effector CAR T cells such that the single cell gene expression data of cytotoxic effector CAR T cells are within the one or more cluster(s) associated with the functional effector T cell functional state.

125. A method for treating a disease or condition in a subject in need thereof, the method comprising administering a preparation of CAR T cells comprising a set of differentially expressed markers.

126. A method for enriching a preparation of CAR T cells for CAR T cells that differentiate into cytotoxic effector cells upon administration to a subject using one or more surface proteins encoded by marker genes of a pre-effector gene signature comprising genes which are expressed higher on cytotoxic effector precursors and lower or not at all on other CAR T cells from the preparation.

127. A preparation of CAR T cells with cytotoxic effector potential enriched by the method of claim 126.

128. Non-transitory computer-readable medium configured to communicate with one or more processor(s) of a computational system, the non-transitory computer-readable medium including instructions thereon, that when executed by the processor(s), cause the computational system to:
a) receive, as inputs,
a preparation of CAR T cell gene expression dataset comprising single cell gene expression data of CAR T cells,
TCR sequencing data each associated with respective single cell gene expression data of the preparation of CAR T cell single cell gene expression dataset,
a post-infusion CAR T cell gene expression dataset comprising single cell gene expression data of CAR T cells originating from subjects post-infusion, and
endogenous TCR sequencing data each associated with respective single cell gene expression data of the post-infusion CAR T cell gene expression dataset;
b) identify, based on the post-infusion CAR T cell gene expression dataset, single cell gene expression data of cytotoxic effector CAR T cells;
c) identify clonal TCR sequencing data such that the clonal TCR sequencing data is (i) associated with single cell gene expression data of the preparation of CAR T cell gene expression dataset, and is (ii) clonally related to endogenous TCR sequencing data associated with each of the single cell gene expression data of cytotoxic effector CAR T cells;
d) identify cytotoxically effective precursor gene expression data such that the cytotoxically effective precursor gene expression data comprises single cell gene expression data of the preparation of CAR T cell gene expression dataset associated with the clonal TCR sequencing data;
e) identify, based at least in part on the cytotoxically effective precursor gene expression data, a pre-effector gene signature comprising markers for determining a cytotoxic effector potential of CAR T cells; and
f) provide, as an output, the pre-effector gene signature.

129. The non-transitory computer-readable medium of claim 128, wherein the instructions at step b) further comprise the following instructions:
cluster the single cell gene expression data of the post-infusion CAR T cell gene expression dataset using a clustering algorithm, resulting in a plurality of clusters such that each cluster in the plurality of clusters comprises similar single cell gene expression data within the cluster and such that single cell gene expression data differs across differing clusters;
determine one or more functional effector cluster(s) of the plurality of clusters such that the one or more functional effector cluster(s) are associated with a functional effector T cell functional state; and
identify the single cell gene expression data of cytotoxic effector CAR T cells such that the single cell gene expression data of cytotoxic effector CAR T cells are within the one or more cluster(s) associated with the functional effector T cell functional state.

130. The non-transitory computer-readable medium of claim 129, wherein the one or more functional effector cluster(s) are determined according to the following instructions:
compare, for each cluster of the plurality of clusters, single cell gene expression data of the cluster to every other cluster to thereby identify differentially expressed markers associated with the cluster; and
determine the one or more functional effector cluster(s) such that differentially expressed markers of each of the one or more functional effector clusters(s) comprise known genes associated with the functional effector T cell functional state.

131. Non-transitory computer-readable medium configured to communicate with one or more processor(s) of a computational system, the non-transitory computer-readable medium including instructions thereon, that when executed by the processor(s), cause the computational system to:
a) receive, as an input, a preparation of CAR T cell gene expression dataset comprising single cell gene expression data of CAR T cells of a preparation of CAR T cells;
b) determine a cytotoxic effector potential for each single cell gene expression data of the preparation of CAR T cell gene expression dataset,
wherein the cytotoxic effector potential indicates likelihood of a CAR T cell, when administered, to give rise to a lineage that results in functional effector post-infusion CAR T cells; and
c) calculate, based on the cytotoxic effector potentials, a gene set module score for the preparation of CAR T cells,
wherein the gene set module score indicates likelihood of the preparation of CAR T cells, when administered, to give rise to a clinically effective quantity of functional effector post-infusion T cells.

132. The non-transitory computer-readable medium of claim 131, wherein the gene set module score is calculated according to the following instructions:
determine a threshold value for the cytotoxic effector potential such that single cell gene expression data determined to have a cytotoxic effector value above the threshold represent CAR T cells having a high likelihood to give rise to a lineage that results in functional effector post-infusion T cells;
calculate a numerator value that is equal to number of CAR T cells of the preparation of CAR T cells represented by the single cell gene expression data having a cytotoxic effector potential above the threshold value;
calculate a denominator value that is equal to a total number of CAR T cells of the preparation represented by the single cell gene expression data of the preparation of CAR T cell gene expression dataset; and
calculate the gene set module score based on a ratio of the numerator value to the denominator value.

133. The non-transitory computer-readable medium of claim 131, wherein the set of differentially expressed markers comprises increased expression of TIGIT and/or LAG3 genes.

134. The non-transitory computer-readable medium of claim 131, wherein the set of differentially expressed markers comprises decreased expression of CD27 gene.

135. The non-transitory computer-readable medium of claim 131, wherein the set of differentially expressed markers are selected from CD62L TIGIT, and CD27.


SEQUENCE LISTING (14 sequences)

SEQ ID NO: 1 - protein, 22 aa, synthetic construct
  REGION 1..22: note = Description of Sequence: CD8a Leader
MALPVTALLLPLALLLHAARPD

SEQ ID NO: 2 - protein, 106 aa, synthetic construct
  REGION 1..106: note = Description of Sequence: FMC63scFv - VL
IQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 3 - protein, 15 aa, synthetic construct
  REGION 1..15: note = Description of Sequence: Linker
GGGGSGGGGSGGGGS

SEQ ID NO: 4 - protein, 120 aa, synthetic construct
  REGION 1..120: note = Description of Sequence: FMC63scFv - VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 5 - protein, 69 aa, synthetic construct
  REGION 1..69: note = Description of Sequence: CD8a Hinge/transmembrane domain
TTTPAPRPPTPAPTIASQPLSLRPEACRPAAGGAVHTRGLDFACDIYIWAPLAGTCGVLLLSLVITLYC

SEQ ID NO: 6 - protein, 42 aa, synthetic construct
  REGION 1..42: note = Description of Sequence: 4-1BB costim domain
KRGRKKLLYIFKQPFMRPVQTTQEEDGCSCRFPEEEEGGCEL

SEQ ID NO: 7 - protein, 112 aa, synthetic construct
  REGION 1..112: note = Description of Sequence: CD3zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - DNA, 63 bp, synthetic construct
  misc_feature 1..63: note = Description of Sequence: CD8a Leader
  source 1..63: mol_type = other DNA
atggccttaccagtgaccgccttgctcctgccgctggccttgctgctccacgccgccaggccg

SEQ ID NO: 9 - DNA, 324 bp, synthetic construct
  misc_feature 1..324: note = Description of Sequence: FMC63scFv - VL
  source 1..324: mol_type = other DNA
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggt

SEQ ID NO: 10 - DNA, 42 bp, synthetic construct
  misc_feature 1..42: note = Description of Sequence: Linker
  source 1..42: mol_type = other DNA
ggcggtggctcgggcggtggtgggtcgggtggcggcggatct

SEQ ID NO: 11 - DNA, 360 bp, synthetic construct
  misc_feature 1..360: note = Description of Sequence: FMC63scFv - VH
  source 1..360: mol_type = other DNA
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 12 - DNA, 207 bp, synthetic construct
  misc_feature 1..207: note = Description of Sequence: CD8a Hinge/transmembrane domain
  source 1..207: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 13 - DNA, 132 bp, synthetic construct
  misc_feature 1..132: note = Description of Sequence: 4-1BB costim domain
  source 1..132: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactgagagtg

SEQ ID NO: 14 - DNA, 330 bp, synthetic construct
  misc_feature 1..330: note = Description of Sequence: CD3zeta
  source 1..330: mol_type = other DNA
aagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc